(19) United States
(12) Patent Application Publication
(10) Pub. No.: US 2025/0135012 A1
(43) Pub. Date: May 1, 2025

(54) DENDRIMER CONJUGATES OF SMALL MOLECULE BIOLOGICS FOR INTRACELLULAR DELIVERY

(71) Applicant: The Johns Hopkins University, Baltimore, MD (US)

(72) Inventors: Kannan Rangaramanujam, Higland, MD (US); Wathsala Liyanage, Ellicott City, MD (US); Tony Wu, Issaquah, WA (US); Sujatha Kannan, Highland, MD (US)

(21) Appl. No.: 18/693,757

(22) PCT Filed: Sep. 21, 2022
(86) PCT No.: PCT/US2022/076775

§ 371 Date: Mar. 20, 2024

Related U.S. Application Data
(60) Provisional application No. 63/246,705, filed on Sep. 21, 2021.

(51) Int. Cl. A61K 47/59; A61K 47/54; A61P 37/06; C12N 15/113

(52) U.S. Cl. CPC A61K 47/595; A61K 47/543; A61K 47/544; A61P 37/06; C12N 15/1136; C12N 2310/141; C12N 2310/351

(57) ABSTRACT

Compositions of hydroxyl-terminated dendrimers covalently conjugated with functional nucleic acids (D-FNA) to prevent, treat or diagnose one or more diseases or disorders in a subject in need thereof, and methods of use thereof, have been developed. Covalent conjugation of FNA to dendrimer greatly enhances serum half-life and bioavailability, protecting the payload from protein adsorption and enzymatic degradation. Preferably, the functional nucleic acids are covalently conjugated to dendrimers by functionally releasable coupling elements for intracellular release of FNAs within activated macrophages, including tumor-associated microglia (TAMs). An exemplary functionally releasable coupling element is a glutathione-sensitive coupling element. Exemplary FNAs include antisense RNAs, microRNAs and silencing RNAs. The compositions are particularly suited for treating or ameliorating symptoms of inflammatory diseases and proliferative diseases. Methods of treating a human subject having or at risk of inflammatory diseases and proliferative diseases are provided.

### CROSS-REFERENCE TO RELATED APPLICATIONS

This application claims the benefit of and priority to U.S. Ser. No. 63/246,705, filed on Sep. 21, 2021, which is incorporated by reference herein in its entirety.

### REFERENCE TO SEQUENCE LISTING

The Sequence Listing submitted as an xml file named “JHU_C_17048_PCT.xml,” created on Sep. 21, 2022, and having a size of 10,664 bytes is hereby incorporated by reference pursuant to 37 C.F.R. § 1.834(c)(1).

### FIELD OF THE INVENTION

The invention is generally in the field of nucleic acid delivery, and in particular, methods for delivering small molecules such as RNA molecules covalently bound to a dendrimer which is selectively taken up at sites or regions in need thereof.

### BACKGROUND OF THE INVENTION

Antisense oligonucleotides (ASOs) and small interfering RNAs (siRNAs) are the two most widely used strategies for silencing gene expression. The potential use of antisense and siRNA oligonucleotides as therapeutic agents has elicited a great deal of interest. However, a major issue for oligonucleotide-based therapeutics involves effective intracellular delivery of the active molecules. Delivering oligonucleotides in whole organisms requires crossing many barriers. Degradation by serum nucleases, clearance by the kidney, or inappropriate biodistribution can prevent the oligonucleotide from ever reaching its target organ. The oligonucleotide must pass through the blood vessel wall and navigate the interstitial space and extracellular matrix. Finally, if the oligonucleotide succeeds in reaching the appropriate cell membrane, it will usually be taken up into an endosome, from which it must escape to be active.

Small interference RNA (siRNA) is an emerging potent therapeutic for central nervous system (CNS) disorders due to its ability to inhibit specific genes that are integral with neurological disease progression. However, successful delivery of siRNA to the brain parenchyma faces obstacles such as the blood-brain barrier and poor cellular uptake. In addition, siRNAs are highly unstable in physiological conditions and are susceptible to protein binding and enzymatic degradation, necessitating a higher dosage to remain effective. Efforts to develop efficient viral and non-viral carriers have been met with challenges of immunogenicity, vehicle toxicity, and aggregation. Further, consistent nucleic acid loading is difficult to achieve in delivery systems that rely on non-covalent interactions.

Therefore, it is an object of the invention to provide compositions for delivery of functional nucleic acids, in particular RNA molecules that are capable of modulating gene expression and/or other biochemical activities in the cell.

It is also an object of the present invention to provide drug delivery formulations for treating diseases, disorders, and injury of the brain and central nervous system, particularly those associated with activated microglia and/or astrocytes.

It is a further object of the present invention to provide biocompatible and inexpensive nanomaterials for targeted or selective delivery of functional nucleic acids, in particular RNA molecules to the central nervous system with little to no local or systemic toxicity.

### SUMMARY OF THE INVENTION

It has been established that hydroxyl-terminated dendrimers can selectively deliver covalently conjugated small molecule biologics such as functional nucleic acids to activated macrophages and microglia and neurons at sites of injury and disease with high efficacy and low toxicity. The dendrimers shield and stabilize the functional nucleic acids in vivo, enabling efficient gene silencing and/or regulation of the expression of targeted genes for the treatment and prevention of diseases and disorders.

Compositions of hydroxyl-terminated dendrimers covalently conjugated to one or more functional nucleic acids, optionally via one or more spacers, are provided. Typically, the functional nucleic acids are conjugated to less than 50% of the terminal OH groups on the surface of the dendrimer. Typically, the one or more functional nucleic acids inhibit the transcription, translation, or function of a target gene. In some embodiments, the one or more functional nucleic acids are antisense molecules, small interfering RNAs (siRNAs), microRNAs (miRNA), aptamers, ribozymes, triplex forming molecules, or external guide sequences. A preferred functional nucleic acid is a siRNA or miRNA. In particular embodiments, the miRNA is miR-126.

The hydroxyl-terminated dendrimers are generally generation 2 (G2), generation 3 (G3), generation 4 (G4), generation 5 (G5), generation 6 (G6), generation 7 (G7), or generation 8 (G8) dendrimers. In preferred embodiments, the dendrimers are poly(amidoamine) (PAMAM) dendrimers. In some embodiments, the dendrimers are covalently conjugated to the one or more functional nucleic acids via one or more spacers. Suitable spacers include one of N-Succinimidyl 3-(2-pyridyldithio)-propionate (SPDP), glutathione, gamma-aminobutyric acid (GABA), polyethylene glycol (PEG). In a preferred embodiment, the dendrimers are covalently conjugated to the one or more functional nucleic acids via disulfide bonding. In some embodiments, the dendrimers are further conjugated to one or more additional therapeutic, prophylactic, and/or diagnostic agents.

Compositions of dendrimers conjugated with functional nucleic acids include one of the following structures:

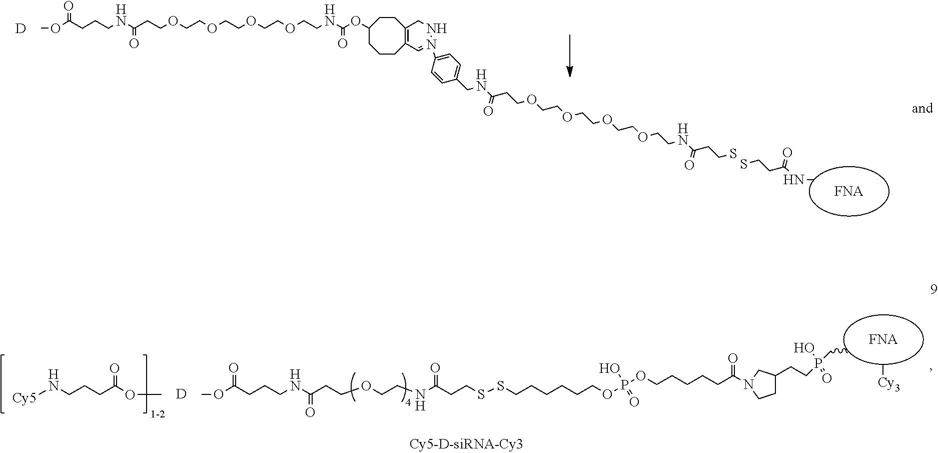

where the circle denoted with D is a hydroxyl-terminated dendrimer and the oval denoted with FNA is a functional nucleic acid.

Pharmaceutical compositions including hydroxyl-terminated dendrimers covalently conjugated to one or more functional nucleic acids, optionally via one or more spacers, and one or more pharmaceutically acceptable excipients are also provided. The pharmaceutical compositions are generally formulated for parenteral or oral administration in a form such as hydrogels, nanoparticle or microparticles, suspensions, powders, tablets, capsules, and solutions.

Methods for treating one or more symptoms of a disease or disorder in a subject in need thereof including administering to the subject an effective amount of a pharmaceutical composition including hydroxyl-terminated dendrimers covalently conjugated to one or more functional nucleic acids, optionally via one or more spacers, and one or more pharmaceutically acceptable excipients, effective to alleviate one or more symptoms of the disease or disorder. Typically, the methods treat or prevent inflammation, proliferative disease such as cancer, or neurological disease in the subject. In some embodiments the methods are for the treatment of inflammation associated with one or more diseases, conditions, and/or injuries of the eye, the brain, and/or the nervous system (CNS). Exemplary diseases, conditions, and/or injuries of the eye that can be treated by the methods are those associated with choroid neovascularization. In some embodiments, the functional nucleic acid is a miRNA specific for vascular endothelial growth factor (VEGF). An exemplary miRNA specific for VEGF is miR-126. In a preferred embodiment, dendrimers covalently conjugated with miR-126 are selectively delivered to the eye to treat or prevent one or more symptoms of macular degeneration in the subject.

In some embodiments the methods deliver one or more functional nucleic acids conjugated to dendrimers for the treatment or prevention of cancer in the subject. Exemplary cancers that can be treated include breast cancer, cervical cancer, ovarian cancer, uterine cancer, pancreatic cancer, skin cancer, multiple myeloma, prostate cancer, testicular germ cell tumor, brain cancer, oral cancer, esophagus cancer, lung cancer, liver cancer, renal cell cancer, colorectal cancer, duodenal cancer, gastric cancer, and colon cancer. Therefore, in some embodiments, the methods deliver an effective amount of functional nucleic acids to reduce tumor size or inhibit tumor growth. In some embodiments the methods administer the dendrimer-functional nucleic acid composition directly into the eye. An exemplary method for administration into the eye is by intravitreal injection. In other embodiments, the composition is administered orally or parenterally. For example, in particular embodiments, the composition is administered intravenously.

The methods typically administer dendrimer-functional nucleic acid compositions at a time selected from once every day, once every other day, once every three days, once a week, once every 10 days, once every two weeks, once every three weeks and once every month. For example, in some embodiments the composition is administered once every two weeks, or less frequently. Typically, the amount of the functional nucleic acid effective to treat the disease or disorder according to the described methods is 50% or less of the amount of the same functional nucleic acid required to treat the disease or disorder in the absence of the dendrimer.

Kits including dendrimer-functional nucleic acid compositions, optionally including reagents, buffers and apparatus for administering the compositions to a subject, and/or instructions for use are also described.

### BRIEF DESCRIPTION OF THE DRAWINGS

FIG. 1 is a schematic showing molecular structures in a stepwise synthetic route for producing functionalized Cy5-D-PEG₄-TCO. The hydroxyl PAMAM dendrimer generation 6 (PAMAM-G6-OH) was treated with 4-tert-butoxycarbonylamino)butyric acid (Boc-protected GABA) linker, (2) and the resulted product, (3) was deprotected using dichloromethane (DCM)/trifluoroacetic acid (TFA) (4:1). The product, (4) was labeled with Cy5 fluorophore using Cy5 N-hydroxysuccinimide (NHS) ester and the resulted intermediate, (5) was conjugated with trans-cyclooctene (TCO) linker, PEG4-TCO to obtain functionalized Cy5-D-PEG₄-TCO (6). The subscripted numbers in the formulas indicate the number of GABA BOC, PEG₄-TCO, or flurophore attached per dendrimer.

FIG. 2 is a schematic showing molecular structures in a stepwise synthetic route for producing dendrimer-Cy5-ASO conjugate, including modification of ASO for conjugation with dendrimer. ASO was first substituted with PEGylated tetrazine using Methyltetrazine-PEG₄-S—S—NHS (8) reagent to form ASO-PEG4-Tz (9), then (9) was reacted with Cy5-D-PEG₄-TCO, (6) to obtain the final product Cy5-D-ASO (10).

FIG. 3 is a schematic showing synthesis of functionalized Cy5-D-PEG₄-SPDP. The hydroxyl PAMAM dendrimer generation 6 (PAMAM-G6-OH) was treated with Boc-protected GABA linker, (2) and the resulting product (3) was deprotected using TFA. The product, (4) was labeled with Cy5 fluorophore and the resulting intermediate, (5) was conjugated with SPDP to obtain functionalized Cy5-D-PEG4-SPDP, (6).

FIG. 4 is a schematic showing synthesis of Cy5-D-siRNA conjugate. The siRNA, (7) is activated by reducing dithiol group using DTT and the resulting product, (8) is reacted with activated Cy5-D-PEG₄-SPDP, 6 to obtain the final product Cy5-D-siRNA, (9).

FIG. 5 is a bar graph of dose dependent knockdown of green fluorescent protein (GFP) expression by D-siGFP in HEK-293T cells, showing Relative Fluorescence (0-1.5) over Dosage (0-500 nm) for each of Control, 24 h, 48 h and 72 hr samples, respectively.

FIG. 6 is a bar graph of D-si-GFP dose response curve 24H, showing Relative Fluorescence (0-2) over Dosage (0-500 nm) for the 24 h samples.

FIGS. 7A-7C are bar graphs showing delivery methods resulting in significant GFP knockdown of HEK-293T cells. Relative fluorescence was obtained using background adjusted intensity in the GFP channel and normalized to 0 h internal control. FIG. 7A is a bar graph of vehicle dependent GFP knockdown showing ˜50 to 100% Knockdown to OH for each of Control, siGFP, RNA/Max LIPOFECTAMINE® 2000, LIPOFECTAMINE® 3000, and D-siGFP samples, for each of 24 or 48 hours, respectively. Data are presented as averages of duplicates±SEM. FIG. 7B is a bar graph of GFP Protein Expression (0-2.5) Control, siGFP, RNA/Max LIPOFECTAMINE® 2000, LIPOFECTAMINE® 3000, and D-siGFP, respectively. Relative expression of GFP was obtained by normalizing GFP expression to Cyclophilin B expression. FIG. 7C is a bar graph of Confluency (0-1.5) over Time (0-48 hr) for each of control, D-siGFP, Lipo2000, Lipo3000, RNAi Max, and siGFP, respectively. Cell viability via confluency did not suggest cytotoxic effects.

FIG. 8 is a bar graph of in vivo GFP knockdown, showing Tumor KD as % of CH (0-80) for each of control, siGFP, D-scRNA, and D-siGFP.

FIG. 9 is a schematic showing synthesis of Dendrimer-miR126 conjugate. The surface of generation 6 hydroxyl-terminated dendrimer is functionalized with a disulfide linker (PDP). Thiol-modified miR-126 is activated by reducing dithiol group using DTT and the resulting product, (8) is reacted with thiol-modified dendrimer to obtain the final product D-miR126.

FIGS. 10A-10C are bar graphs showing the relative mRNA expression levels of TNFα (FIG. 10A) and IL-1β (FIG. 10B) in BV2 cells in an untreated control group and in experimental groups stimulated with LPS in the presence of D-miR126 and miR-126 at a concentration of 1 nM, 5 nM, 10 nM, and 100 nM; and the relative mRNA expression levels of VEGF-A in HMECs (FIG. 10C) in an untreated control group and in experimental groups treated with D-miR126 and miR-126 at a concentration of 1 nM, 5 nM, 10 nM, and 100 nM.

FIGS. 11A-11D are bar graphs showing total length (FIG. 11A), number of isolated segments (FIG. 11B), total enclosed area (FIG. 11C), and number of nodes and pieces (FIG. 11D), of the cell networks formed by HMECs, in an untreated control group and in experimental groups treated with D-miR126 at a concentration of 1 nM, 5 nM, 10 nM, and 100 nM; or miR-126 at a concentration of 10 nM and 100 nM, based on Matrigel-based tube formation assays.

FIGS. 12A-12B are bar graphs showing CNV area stained with isolectin antibodies and quantified through fluorescence microscopy in an untreated control or in experimental groups treated with D-mirR126 at a concentration of 0.1 μg/μL, 1 μg/μL, and 2 μg/μL, or with miR-126 at a concentration of 1 μg/μL at 7 days post-CNV (FIG. 12A) and 14 days post-CNV (FIG. 12B).

FIGS. 13A-13D are bar graphs showing relative VEGF-A protein expression levels measured with ELISA in an untreated control or in experimental groups treated with D-mirR126 at a concentration of 0.1 μg/μL, and 1 μg/μL, or with miR-126 (FIG. 13A); and the relative mRNA expression levels of VEGF-A in mice treated with PBS (control group) and in experimental groups treated with D-miR126 or miR-126 (FIG. 13B); and the relative mRNA expression levels of TNFα (FIG. 13C) and IL-1β (FIG. 13D) in mice treated with PBS (control group) and in experimental groups treated with D-miR126 or miR-126.

FIGS. 14A-14D are bar graphs showing percentage of colocalization of Cy3 and Cy5 signals with isolectin GS-IB4 staining (blood vessel+macrophage) and Iba1 (macrophage) staining at 1, 3, 5, 7, and 14 days after administration of miR-126 (FIG. 14A), Cy3 colocalization after administration of D-miR126 (FIG. 14B), Cy5 colocalization after administration of D-miR126 (FIG. 14C), as well as colocalization between dendrimer (Cy5) and miR-126 (Cy3) as a measure of payload release in vivo (FIG. 14D).

FIG. 15 is a schematic showing synthesis of Dendrimer-ALG1001. Surface of generation 6 hydroxyl-terminated dendrimer is functionalized with alkyne terminated linkers. ALG-1001 peptide is then attached using copper catalyzed click reaction to yield D-ALG conjugates.

FIG. 16 is a bar graph showing metrics extracted for the integrity and expanse of vessel formation showing the number of times vessels intersect one another (junctions), the number of spaces enclosed by vessels (meshes), the number of connected vessels (segments), and the number of isolated vessels (isolated segments) in an untreated control group and in experimental groups treated with D-ALG and ALG-1001 at a concentration of 1 mM, 100 nM, and 10 nM.

FIG. 17 is a bar graph of relative protein expression of MAPL, FAK phosphorylated MAPK (p-MAPL), phosphorylated FAK (p-FAK) in response to VEGF stimulation in an untreated control group and in experimental groups treated with D-ALG and ALG-1001 at a concentration of 1 mM and 100 nM, determined by protein bands associated with ERK (42 and 44 kDa) and FAK (110 kDa) from Western blotting analysis and normalized to an internal control (cyclophilin B).

FIGS. 18A-18B are bar graphs showing the relative expression of pro-inflammatory cytokine IL1β (FIG. 18A) and TNFα (FIG. 18B) produced by RAW264.7 cells in response to LPS stimulation after pretreatment with ALG-1001 and D-ALG1001. P-values denoted here compares the level of IL1β and TNFα expression to untreated controls.

FIGS. 19A-19B are bar graphs showing CNV area stained with isolectin antibodies and quantified through fluorescence microscopy in an untreated control or in experimental groups intraperitoneally dosed once every 4 days at 150 μg peptide basis with D-ALG1001 (150 μg) or with ALG-1001 (150 μg) at 7 days post-CNV (FIG. 19A) and 14 days post-CNV (FIG. 19B).

FIGS. 20A-20D are bar graphs showing protein quantity (U/ml) of FAK (FIG. 20A), phospho-FAK (Y397) (FIG. 20B), p44/42 ERK (FIG. 20C), phospho-p44/42 ERK (FIG. 20D) as determined by ELISA.

FIGS. 21A-21C are bar graphs showing the relative mRNA expression levels of VEGF-A (FIG. 21A), TNFα (FIG. 21B), and IL-1β (FIG. 21C) in animals treated with PBS (control group) and in experimental groups treated with D-ALG and ALG-1001.

FIG. 22 is a schematic showing synthesis of G1-Glucose. Stepwise synthesis of G1-glucose; the hexapropagylated core 1, was treated treated with AB4 building block (β-Glucose-PEG₄-azide), 2 under clasical click regents (CuAAC click reaction), catalytic amount of copper sulfate pentahydrate (CuSO₄·5H₂O) and sodium ascorbate in DMF:H₂O (1:1) to produce G1-glucose-24-OAc, 3. Then compound 3 was treated under typical Zemplén conditions (to remove acetate groups) to obtain the desired product 4 (G1-glucose).

FIG. 23 is a schematic showing synthesis of Glu-G2 dendrimer. Stepwise synthesis of G2-glucose; G1-glucose dendrimer 4 was treated with sodium hydride (60% dispersion in mineral oil) for 15 min at 0° C. and then treated with Propargyl bromide (80% w/w solution in toluene). The reaction was stirred at room temperature for 8 h to form compound 5. The compound 5 next treated with AB4 building block (β-Glucose-PEG₄-azide), 2 under clasical click regents (CuAAC click reaction), catalytic amount of copper sulfate pentahydrate (CuSO₄·5H₂O) and sodium ascorbate in DMF:H₂O (1:1) to produce G2-glucose-96-OAc, 6. And then compound 6 was reacted under typical Zemplén conditions to obtain the desired product 7 (G2-glucose).

FIG. 24 is a schematic showing synthesis of Cy5-Glu-G2-PEG₄-SPDP. The Glu-G2 dendrimer was treated with NaH and propargyl bromide and the resulted product, 2 was further reacted with N₃-PEG3-amine, 3 using CUAAC click condition to form compound 4. The product, 4 was labeled with Cy5 fluorophore and the resulted intermediate, 5 was conjugated with SPDP to obtain functionalized Cy5-Glu-G2-PEG4-SPDP, 6. The subscripted numbers in the formulas indicate the number of attachments per dendrimer.

FIG. 25 is a schematic showing synthesis of Cy5-Glu-G2-siRNA conjugate. The siRNA, 7 was activated by reducing dithiol group using DTT and the resulted product, 8 was reacted with activated Cy5-Glu-G2-PEG₄-SPDP, 6 to obtain the final product, Cy5-Glu-G2-siRNA, 9.

### DETAILED DESCRIPTION OF THE INVENTION

### I. Definitions

The terms “active agent” or “biologically agent” are therapeutic, prophylactic or diagnostic agents used interchangeably to refer to a chemical or biological compound that induces a desired pharmacological and/or physiological effect, which may be prophylactic, therapeutic or diagnostic. These may be a nucleic acid, a nucleic acid analog, a small molecule having a molecular weight less than 2 kD, more typically less than 1 kD, a peptidomimetic, a peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs.

The term “nucleotide” refers to a molecule that contains a base moiety, a sugar moiety, and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The terms “oligonucleotide” or a “polynucleotide” are synthetic or isolated nucleic acid polymers including a plurality of nucleotide subunits. The terms “nucleic acid,” “polynucleotide,” and “oligonucleotide” are interchangeable and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones, locked nucleic acid). In general, and unless otherwise specified, an analogue of a particular nucleotide has the same base-pairing specificity, i.e., an analogue of A will base-pair with T.

The term “pharmaceutically acceptable salts” is art-recognized, and includes relatively non-toxic, inorganic, and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine;

The term “therapeutic agent” refers to an agent that can be administered to treat one or more symptoms of a disease or disorder.

The term “diagnostic agent” generally refers to an agent that can be administered to reveal, pinpoint, and define the localization of a pathological process. The diagnostic agents can label target cells that allow subsequent detection or imaging of these labeled target cells. In some embodiments, diagnostic agents can, via dendrimer or suitable delivery vehicles, target/bind activated microglia in the central nervous system (CNS).

The term “prophylactic agent” generally refers to an agent that can be administered to prevent disease or to prevent certain conditions, such as a vaccine.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders, such as reducing, preventing, or reversing the learning and/or memory deficits in an individual suffering from Alzheimer's disease etc. In one or more neurological or neurodegenerative diseases, an effective amount of the drug may have the effect of stimulation or induction of neural mitosis leading to the generation of new neurons, i.e., exhibiting a neurogenic effect; prevention or retardation of neural loss, including a decrease in the rate of neural loss, i.e., exhibiting a neuroprotective effect. An effective amount can be administered in one or more administrations.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more inhibitors may inhibit or reduce the activity and/or quantity of nSMase2 associated activated microglia by about 10%, 20%, 30%, 40%, 50%, 75%, 85%, 90%, 95%, or 99% from the activity and/or quantity of the same cells in equivalent tissues of subjects that did not receive or were not treated with the dendrimer compositions. In some embodiments, the inhibition and reduction are compared to mRNAs, proteins, cells, tissues, and organs levels. For example, an inhibition and reduction in choroidal neovascularization in the eye, as compared to an untreated control subject.

The term “treating” or “preventing” a disease, disorder, or condition from occurring in an animal which may be predisposed to the disease, disorder and/or condition but which has not yet been diagnosed as having it; inhibiting progress of the disease, disorder, or condition, and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with brain tumors are mitigated or eliminated, including, but are not limited to, reducing the rate of tumor growth, decreasing symptoms resulting from the disease, increasing the quality of life of those suffering from the disease, decreasing the dose of other medications required to treat the disease, delaying the progression of the disease, and/or prolonging survival of individuals.

The term “biodegradable” generally refers to a material that will degrade or erode under physiologic conditions to smaller units or chemical species that are capable of being metabolized, eliminated, or excreted by the subject. The degradation time is a function of composition and morphology.

The term “dendrimer” includes, but is not limited to, a molecular architecture with an interior core, interior layers (or “generations”) of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific locale. The moiety may be, for example, a protein, nucleic acid, nucleic acid analog, carbohydrate, or small molecule. The entity may be, for example, a therapeutic compound such as a small molecule, or a diagnostic entity such as a detectable label. The locale may be a tissue, a particular cell type, or a subcellular compartment. In one embodiment, the targeting moiety directs the localization of an agent. In a preferred embodiment, the dendrimer composition selectively targets activated microglia in the absence of an additional targeting moiety.

The term “prolonged residence time” refers to an increase in the time required for an agent to be cleared from a patient's body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types.

The terms “incorporated” and “encapsulated” refer to incorporating, formulating, or otherwise including an agent into and/or onto a composition that allows for release, such as sustained release, of such agent in the desired application. The agent or other material can be incorporated into a dendrimer, by binding to one or more surface functional groups of such dendrimer (by covalent, ionic, or other binding interaction), by physical admixture, by enveloping the agent within the dendritic structure, and/or by encapsulating the agent inside the dendritic structure.

### II. Compositions

Dendrimer complexes suitable for delivering one or more small molecule biologics, particularly one or more functional nucleic acids to prevent, treat, or diagnose one or more diseases or conditions have been developed.

Compositions of dendrimer complexes include one or more prophylactic, or therapeutic agents for treating or preventing one or more diseases or disorders covalently conjugated with the dendrimers. Generally, one or more active agent is conjugated to the dendrimer complex at a concentration of about 0.01% to about 50%, preferably about 1% to about 30%, more preferably about 5% to about 20% by weight of the total dendrimer/active agent complex. Preferably, one or more agent is covalently conjugated to the dendrimer via one or more linkages such as disulfide, ester, ether, thioester, carbamate, carbonate, hydrazine, and amide, optionally via one or more spacers. Exemplary agents include small molecule biologics, such as functional nucleic acid molecules.

The presence of the additional agents can affect the zeta-potential or the surface charge of the particle. In one embodiment, the zeta potential of the dendrimers is between about −100 mV and about 100 mV, between about −50 mV and about 50 mV, between about −25 mV and about 25 mV, between about −20 mV and about 20 mV, between about −10 mV and about 10 mV, between about −10 mV and about 5 mV, between about −5 mV and about 5 mV, between about −2 mV and about 2 mV, or between about −1 mV and about 1 mV, inclusive. In a preferred embodiment, the surface charge is neutral or near neutral. The range above is inclusive of all values from −100 mV to 100 mV.

### A. Dendrimers

Dendrimers are three-dimensional, hyperbranched, monodispersed, globular and polyvalent macromolecules comprising a high density of surface end groups (Tomalia, D. A., et al., Biochemical Society Transactions, 35, 61 (2007); and Sharma, A., et al., ACS Macro Letters, 3, 1079 (2014)). Due to their unique structural and physical features, dendrimers are useful as nanocarriers for various biomedical applications including targeted drug/gene delivery, imaging and diagnosis (Sharma, A., et al., RSC Advances, 4, 19242 (2014); Caminade, A.-M., et al., Journal of Materials Chemistry B, 2, 4055 (2014); Esfand, R., et al., Drug Discovery Today, 6, 427 (2001); and Kannan, R. M., et al., Journal of Internal Medicine, 276, 579 (2014)).

Recent studies have shown that dendrimer surface groups have a significant impact on their biodistribution (Nance, E., et al., Biomaterials, 101, 96 (2016)). Hydroxyl terminated generation 4 PAMAM dendrimers (˜4 nm size) without any targeting ligand cross the impaired BBB upon systemic administration in a rabbit model of cerebral palsy (CP) significantly more (>20-fold) as compared to healthy controls, and selectively target activated microglia and astrocytes (Lesniak, W. G., et al., Mol Pharm, 10 (2013)).

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

Generally, dendrimers have a diameter between about 1 nm and about 50 nm, more preferably between about 1 nm and about 20 nm, between about 1 nm and about 10 nm, or between about 1 nm and about 5 nm. In some embodiments, the diameter is between about 1 nm and about 2 nm. Conjugates are generally in the same size range, although large proteins such as antibodies may increase the size by 5-15 nm. In general, agent is conjugated in a mass ratio of agent to dendrimer of between 0.1:1 and 4:1, inclusive. In preferred embodiments, the dendrimers have a diameter effective to penetrate brain tissue and to retain in target cells for a prolonged period of time.

In some embodiments, dendrimers have a molecular weight between about 500 Daltons and about 100,000 Daltons, preferably between about 500 Daltons and about 50,000 Daltons, most preferably between about 1,000 Daltons and about 20,000 Dalton.

Suitable dendrimers scaffolds that can be used include poly(amidoamine), also known as PAMAM, or STARBURST™ dendrimers; polypropylamine (POPAM), polyethylenimine, polylysine, polyester, iptycene, aliphatic poly(ether), and/or aromatic polyether dendrimers. The dendrimers can have carboxylic, amine and/or hydroxyl terminations. In preferred embodiments, the dendrimers have hydroxyl terminations. Each dendrimer of the dendrimer complex may be same or of similar or different chemical nature than the other dendrimers (e.g., the first dendrimer may include a PAMAM dendrimer, while the second dendrimer may be a POPAM dendrimer).

The term “PAMAM dendrimer” means poly(amidoamine) dendrimer, which may contain different cores, with amidoamine building blocks, and can have carboxylic, amine and hydroxyl terminations of any generation including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. In the preferred embodiment, the dendrimers are soluble in the formulation and are generation (“G”) 4, 5 or 6 dendrimers. The dendrimers may have hydroxyl groups attached to their functional surface groups.

Methods for making dendrimers are known to those of skill in the art and generally involve a two-step iterative reaction sequence that produces concentric shells (generations) of dendritic β-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. Preferable, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of combined targeting moieties, if any, and agents.

### 1. Hydroxyl-Terminated Dendrimers

In some embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols.

In some embodiments, the high-density hydroxyl groups-containing dendrimers are oligo ethylene glycol (OEG)-like dendrimers. For example, a generation 2 OEG dendrimer (D2-OH-60) can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in International Patent Publication No. WO2019094952. In some embodiments, the dendrimer backbone has non-cleavable polyether bonds throughout the structure to avoid the disintegration of dendrimer in vivo and to allow the elimination of such dendrimers as a single entity from the body (non-biodegradable).

In some embodiments, the dendrimer specifically targets a particular tissue region and/or cell type, preferably activated macrophages, such as activated microglia in the CNS. In preferred embodiments, the dendrimer specifically targets a particular tissue region and/or cell type without a targeting moiety. For example, it has been established that hydroxyl (—OH) terminated dendrimers can traverse the blood-brain-barrier (BBB) and permeate into/throughout the brain tissue to be selectively internalized within activated microglia within regions of inflammation in the brain.

Therefore, in preferred embodiments, the dendrimers have a plurality of hydroxyl (—OH) groups on the periphery of the dendrimers. The preferred surface density of hydroxyl (—OH) groups is at least 1 OH group/nm² (number of hydroxyl surface groups/surface area in nm²). For example, in some embodiments, the surface density of hydroxyl groups is more than 2, 3, 4, 5, 6, 7, 8, 9, 10; preferably at least 10, 15, 20, 25, 30, 35, 40, 45, 50, or more than 50. In further embodiments, the surface density of hydroxyl (—OH) groups is between about 1 and about 50, preferably 5-20 OH group/nm² (number of hydroxyl surface groups/surface area in nm²) while having a molecular weight of between about 500 Da and about 10 kDa.

In some embodiments, the dendrimers may have a fraction of the hydroxyl groups exposed on the outer surface, with the others in the interior core of the dendrimers. In preferred embodiments, the dendrimers have a volumetric density of hydroxyl (—OH) groups of at least 1 OH group/nm³ (number of hydroxyl groups/volume in nm³). For example, in some embodiments, the volumetric density of hydroxyl groups is 2, 3, 4, 5, 6, 7, 8, 9, 10, or more than 10, 15, 20, 25, 30, 35, 40, 45, and 50. In some embodiments, the volumetric density of hydroxyl groups is between about 4 and about 50 groups/nm³, preferably between about 5 and about 30 groups/nm³, more preferably between about 10 and about 20 groups/nm³.

In some embodiments, the dendrimer specifically targets a particular tissue region and/or cell types following administration into the body. In preferred embodiments, the dendrimer specifically targets a particular tissue region and/or cell type without a targeting moiety.

### 2. Glucose-Based Dendrimers

In some embodiments, the dendrimers have a hypercore (e.g., dipentaerythritol) and one or more monosaccharide branching units. In some embodiments, the monosaccharide branching units are conjugated to the core or the prior layer of monomers via linkers such as polyethylene glycol chains. In preferred embodiments, the hypercore is dipentaerythritol and the monosaccharide branching unit is glucose-based branching unit.

In further embodiments, spacer molecules can also be alkyl (CH₂)ₙ— hydrocarbon-like units. The branching units are the PEG or alkyl chain linkers between different dendrimer generations, for example, the glucose layers are connected via PEG linkers and triazole rings.

Dendrimers synthesized using glucose building blocks, with a surface made predominantly of glucose moieties, enable targeted delivery to select cells including injured neurons, ganglion cells and other neuronal cells in the brain and the eye.

In one embodiment, the glucose-based dendrimer selectively targets or enriched inside neurons, specifically the nucleus of neurons. In a preferred embodiment, the glucose-based dendrimer selectively targets or enriched inside injured, diseased, and/or hyperactive neurons.

In preferred embodiments, the dendrimers include an effective number of terminal glucose and/or hydroxyl groups for targeting to one or more neurons of the CNS, or the eye. The hydroxyl groups on the dendrimer surface are part of glucose molecules. There are no extra hydroxyls in addition to the glucose molecules on the surface. The number of sugar molecules on the surface is determined by the generation number. All generations are expected to target neurons.

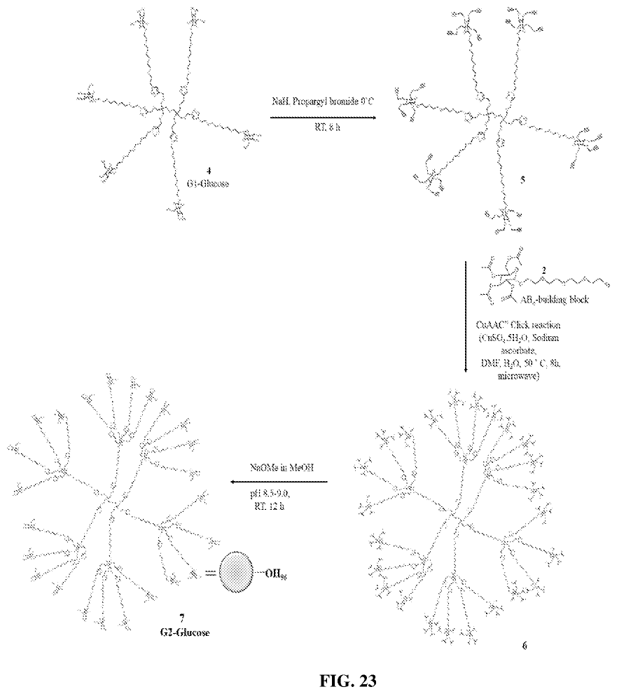

In some embodiments, dendrimers are made of glucose and oligoethylene glycol building blocks. Exemplary glucose dendrimers are shown in the Examples, for example, a generation 1 dendrimer as shown in FIG. 22, and a generation 2 dendrimer as shown in FIG. 23. Some exemplary glucose dendrimers include a generation 1 glucose dendrimer having 24 hydroxyl (—OH) end groups, a generation 2 glucose dendrimer having 96 hydroxyl (—OH) end groups, a generation 3 glucose dendrimer having 396 hydroxyl (—OH) end groups, and generation 4 glucose dendrimer having 1584 hydroxyl (—OH) end groups. In a preferred embodiment, the glucose dendrimer is a generation 2 glucose-based dendrimer that has 24 glucose molecules at the periphery and 6 embedded glucose molecules in the backbone held together by PEG segments.

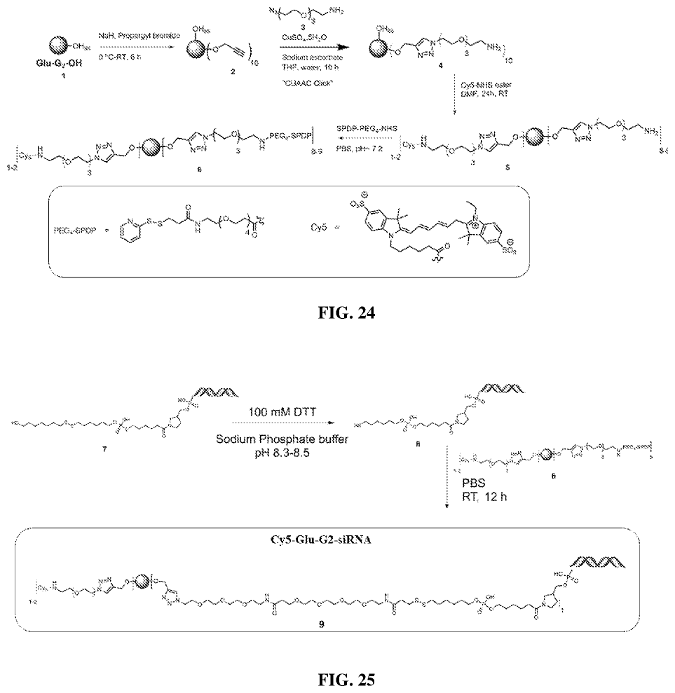

In some embodiments, glucose dendrimers are functionalized for conjugation to additional moieties, for example via SPDP and one or more PEG segments as shown in FIG. 24. In some embodiments, glucose dendrimers are conjugated to siRNA as shown in FIG. 25.

Thus, in some embodiments, one or more functional nucleic acids are conjugated to glucose dendrimers for selective targeting or enrichment inside injured, diseased, and/or hyperactive neurons. Exemplary functional nucleic acids are antisense molecules, small interfering RNAs (siRNAs), microRNAs (miRNA), aptamers, ribozymes, triplex forming molecules, or external guide sequences. A preferred functional nucleic acid is a siRNA or miRNA. In particular embodiments, the miRNA is miR-126.

### B. Coupling Agents and Spacers

Dendrimer complexes are formed of small molecule biologics conjugated to a dendrimer, a dendritic polymer or a hyperbranched polymer via one or more spacers/linkers. Typically, the active agents are coupled to the dendrimer via one or more linkages such as disulfide, ester, carbonate, carbamate, thioester, hydrazine, hydrazides, and amide linkages. In preferred embodiments, the one or more spacers/linkers between a dendrimer and an agent is designed to provide a releasable form of the dendrimer-active complexes in vivo. In some embodiments, the attachment occurs via an appropriate spacer that provides an ester bond between the agent and the dendrimer. In some embodiments, the attachment occurs via an appropriate spacer that provides an amide bond between the agent and the dendrimer. In preferred embodiments, one or more spacers/linkers between a dendrimer and an agent are added to achieve desired and effective release kinetics in vivo.

The term “spacers” includes compositions used for linking an active agent (e.g., functional nucleic acid) to the dendrimer. The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations. The spacer can be chosen from among a class of compounds terminating in sulfhydryl, thiopyridine, succinimidyl, maleimide, vinylsulfone and carbonate group.

In preferred embodiments, the spacer include a thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP.

In other embodiments, the spacer includes peptides, where the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. The spacer can be thiosalicylic acid and its derivatives, (4-succinimidyloxycarbonyl-methyl-alpha-2-pyridylthio)toluene, (3-[2-pyridithio]propionyl hydrazide, The spacer can have maleimide terminations wherein the spacer includes polymer or small chemical entity such as bis-maleimido diethylene glycol and bis-maleimido triethylene glycol, Bis-Maleimidoethane, bismaleimidohexane. The spacer can include vinylsulfone such as 1,6-Hexane-bis-vinylsulfone. The spacer can include thioglycosides such as thioglucose. The spacer can be reduced proteins such as bovine serum albumin and human serum albumin, any thiol terminated compound capable of forming disulfide bonds. The spacer can include polyethylene glycol having sulfhydryl, thiopyridine, maleimide, succinimidyl, and thiol terminations.

The active agents can be either covalently attached or intra-molecularly dispersed or encapsulated. In preferred embodiments, the active agents are covalently attached to the dendrimers. The dendrimer is preferably a PAMAM dendrimer up to generation 10, having carboxylic, hydroxyl, or amine terminations. In preferred embodiments, the dendrimer is a hydroxyl terminated PAMAM dendrimer linked to active agents via a spacer ending in disulfide bonds.

### 1. In Vivo Releasable Linkers

In preferred embodiments, the one or more small-molecule active agents are covalently conjugate to the dendrimer via an in vivo releasable linker. Typically, the covalent linkage to a dendrimer stabilizes the active agents, enhancing the serum half-life of the agent in vivo and preventing enzymatic degradation, whilst maintaining the active agent in a non-functional form. In some embodiments, the linker is designed and selected such that the active agent is released from the covalent attachment with the dendrimer at a pre-determined time or in vivo location, for example, within the intracellular environment. Therefore, in some forms, small molecule biologics such as functional nucleic acids are maintained in a stable and protected but functionally inactive form in the serum, but are released upon internalization into a cell, to become functional. Therefore, in some embodiments, dendrimer/small molecule biologics include an in vivo releasable linker that releases the active agent from the dendrimer, for example by splitting a disulfide bond between the dendrimer and the agent. In some embodiments, the in vivo releasable linker is sensitive to one or more of protease activity, pH, and glutathione concentration. The glutathione concentration release strategy utilizes higher intracellular glutathione concentrations than in plasma. Therefore, the disulfide-containing linker releases cytotoxins after reduction by glutathione. Exemplary glutathione-sensitive linkers are N-Succinimidyl 3-(2-pyridyldithio)-propionate (SPDP), Glutathione (GSH), and Gamma-aminobutyric acid (GABA). An exemplary protease sensitivity strategy utilizes predominant proteases found in lysosomes of tumor cells to recognize and cleave specific peptide sequences in the linker, such as the valine-citrulline (VC) dipeptide as an intracellular cleavage mechanism by cathepsin B. The acid-sensitive strategy is to use a lower pH of the endosome (pH=5-6) and lysosome (pH=4.8) compartments compared to the cytoplasm (pH=7.4) to trigger the hydrolysis of an acid-labile group within the linker, such as a hydrazone.

In a preferred embodiment, the linker releases the small molecule biologics from the dendrimer within the intracellular environment, such that the activity of the small molecule is restricted to the interior of the target cell. In an exemplary embodiment the dendrimer complex includes an OH-terminated PAMAM dendrimer covalently bound to one or more small molecule biologics such as a functional nucleic acid, via a glutathione releasable linker, such as a SPDP linker.

### C. Small Molecule Biologics

The dendrimers are covalently linked to one or more small molecule biologics. The term biologics covers diverse selection of compounds with biological origins, e.g., peptides, nucleic-acid-based compounds, cytokines, replacement enzymes, various recombinant proteins, and monoclonal antibodies. In preferred embodiments, small molecule biologics include siRNAs, oligonucleotides, microRNAs and therapeutic proteins. Small molecule biologics have a molecular weight less than 50,000 amu, preferably less than 20,000 amu, more preferably 5,000-15,000 Dalton. In some embodiments, the small molecule biologics associated with or conjugated to the dendrimer include one or more functional nucleic acids.

### 1. Functional Nucleic Acids

Functional nucleic acids that inhibit the transcription, translation, or function of a target gene are described. Functional nucleic acids are nucleic acid molecules that have a specific function, such as binding a target molecule or catalyzing a specific reaction. As discussed in more detail below, functional nucleic acid molecules can be divided into the following categories: antisense molecules, siRNA, miRNA, aptamers, ribozymes, triplex forming molecules, RNAi, and external guide sequences. The functional nucleic acid molecules can act as effectors, inhibitors, modulators, or stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the target polypeptide itself. Functional nucleic acids are often designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. Therefore, the compositions can include one or more functional nucleic acids designed to reduce expression or function of a target protein.

Methods of making and using vectors for in vivo expression of the described functional nucleic acids such as antisense oligonucleotides, siRNA, shRNA, miRNA, EGSs, gRNA, sgRNA, ribozymes, and aptamers are known in the art. Administering a functional nucleic acid to a subject as a dendrimer-functional nucleic acid complex typically enhances the serum half-life of the functional nucleic acid as compared to the serum half-life of the functional nucleic acid administered alone. In some embodiments conjugation with a dendrimer shields a functional nucleic acid from enzymatic or proteolytic degradation, and prevents non-specific cellular uptake and/or activity of the functional nucleic acid.

Typically, conjugation with a dendrimer will direct in vivo distribution of a functional nucleic acid to one or more sites that are targeted by the dendrimer complex. For example, conjugation with a OH-terminated dendrimer will direct in vivo distribution of a functional nucleic acid to one or more sites of inflammation following systemic administration. In a particular embodiment, conjugation with a OH-terminated dendrimer directs in vivo distribution of a functional nucleic acid to one or more sites of neuroinflammation or neurological damage within the brain and/or CNS following systemic administration.

a. Antisense Oligonucleotides

In some embodiments, the functional nucleic acids are antisense oligonucleotides. Antisense oligonucleotides are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively, the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (Kd) less than or equal to 10⁻⁶, 10⁻⁸, 10⁻¹⁰, or 10⁻¹².

b. Silencing RNA (RNA Interference)

In some embodiments, the functional nucleic acids induce gene silencing through RNA interference (siRNA). Expression of a target gene can be effectively silenced in a highly specific manner through RNA interference.

An RNA polynucleotide with interference activity of a given gene will down-regulate the gene by causing degradation of the specific messenger RNA (mRNA) with the corresponding complementary sequence and preventing the production of protein (see Sledz and Williams, Blood, 106(3):787-794 (2005)). When an RNA molecule forms complementary Watson-Crick base pairs with an mRNA, it induces mRNA cleavage by accessory proteins. The source of the RNA can be viral infection, transcription, or introduction from exogenous sources.

Gene silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme called Dicer, into double stranded small interfering RNAs (siRNA) 21-23 nucleotides in length that contain 2 nucleotide overhangs on the 3′ ends (Elbashir, et al., Genes Dev., 15:188-200 (2001); Bernstein, et al., Nature, 409:363-6 (2001); Hammond, et al., Nature, 404:293-6 (2000); Nykanen, et al., Cell, 107:309-21 (2001); Martinez, et al., Cell, 110:563-74 (2002)). The effect of iRNA or siRNA or their use is not limited to any type of mechanism.

In one embodiment, a siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme Dicer (Elbashir, et al., Nature, 411:494-498 (2001)) (Ui-Tei, et al., FEBS Lett, 479:79-82 (2000)). siRNA can be chemically or in vitro-synthesized or can be the result of short double-stranded hairpin-like RNAs (shRNAs) that are processed into siRNAs inside the cell. For example, WO 02/44321 describes siRNAs capable of sequence-specific degradation of target mRNAs when base-paired with 3′ overhanging ends, and is herein specifically incorporated by reference for the method of making these siRNAs. Synthetic siRNAs are generally designed using algorithms and a conventional DNA/RNA synthesizer. Suppliers include Ambion (Austin, Texas), ChemGenes (Ashland, Massachusetts), Dharmacon (Lafayette, Colorado), Glen Research (Sterling, Virginia), MWB Biotech (Esbersberg, Germany), Proligo (Boulder, Colorado), and Qiagen (Vento, The Netherlands). siRNA can also be synthesized in vitro using kits such as Ambion's SILENCER® siRNA Construction Kit.

Therefore, in some embodiments, the dendrimer includes one or more siRNAs, or one or more vectors expressing an siRNA. The production of siRNA from a vector is more commonly done through the transcription of a short hairpin RNAse (shRNAs). Kits for the production of vectors including shRNA are available, such as, for example, Imgenex's GENESUPPRESSOR™ Construction Kits and Invitrogen's BLOCK-IT™ inducible RNAi plasmid and lentivirus vectors. In some embodiments, the functional nucleic acid is siRNA, shRNA, or miRNA.

i. Micro RNAs (miRNA)

In some embodiments the silencing RNA is a micro RNA (miRNA). MicroRNAs (miRNAs) are a class of non-coding RNAs that play important roles in regulating gene expression. miRNA binds to target sequences reducing the expression of the target gene. miRNA can bind either directly to DNA preventing transcription or to transcribed mRNA preventing translation and directing the mRNA for degradation.

miRNAs are small non-coding RNAs, with an average 22 nucleotides in length. Most miRNAs are transcribed from DNA sequences into primary miRNAs (pri-miRNAs) and processed into precursor miRNAs (pre-miRNAs) and mature miRNAs. In most cases, miRNAs interact with the 3′ untranslated region (3′ UTR) of target mRNAs to induce mRNA degradation and translational repression. However, interaction of miRNAs with other regions, including the 5′ UTR, coding sequence, and gene promoters, have also been reported. Under certain conditions, miRNAs can also activate translation or regulate transcription. The interaction of miRNAs with their target genes is dynamic and dependent on many factors, such as subcellular location of miRNAs, the abundancy of miRNAs and target mRNAs, and the affinity of miRNA-mRNA interactions. miRNAs can be secreted into extracellular fluids and transported to target cells via vesicles, such as exosomes, or by binding to proteins, including Argonautes. Extracellular miRNAs function as chemical messengers to mediate cell-cell communication (O'Brien et al, Front. Endocrinol., 9, pp. 402 (2018)).

In most cases, miRNAs interact with the 3′ UTR of target mRNAs to suppress expression, or interact with other regions, including the 5′ UTR, coding sequence, and gene promoters. miRNAs are also shuttled between different subcellular compartments to control the rate of translation, and even transcription.

Dendrimers covalently attached to miRNAs via one or more releasable linkers are described. In a preferred embodiment, the dendrimer is a OH-terminated PAMAM dendrimer of generation G2-G10, covalently attached to one or more miRNAs via a releasable linker for intracellular release of the miRNA within a target cell, such as an activated macrophage or microglia cell.

(1) miR-126

In some embodiments the micro RNA (miRNA) is the miR-126 miRNA. miR-126 is a human microRNA that is expressed only in endothelial cells, throughout capillaries as well as larger blood vessels, and acts upon various transcripts to control angiogenesis. miR-126 is located within the 7th intron of the EGFL7 gene which resides on human chromosome 9 (Meister et al., Scientific World Journal. 10: 2090-100. doi:10.1100/tsw.2010.198 (2010)).

miR-126 is regulated by the binding of two transcription factors: ETS1 and ETS2, binding of which induces transcription of the miR-126 pre-miRNA, resulting in the formation of the hairpin pri-miRNA. Hairpin miRNA is targeted to Dicer for cleavage, producing mature miR-126 and miR-126* transcripts. Epigenetic regulation of the host gene by accumulation of methylation and gene silencing nucleosomes reduces expression of intronic miRNA. This has been observed in cancers which benefit from the silencing of both EGFL7 and miR-126, resulting in neither being expressed.

One of the main targets of miR-126 is the host gene EGFL7. Mature miR-126 binds to a complementary sequence within EGFL7 preventing translation of the mRNA resulting in a decrease of EGFL7 protein levels. EGFL7 is known to be involved in cell migration and blood vessel formation, making EGFL7 and miR-126 opportune targets for disease, such as cancers, which require the continual formation of blood vessels to supply the tumor with nutrients and cell migration pathways to mediate tissue invasion. Targets of miR-126 include CRK, (a protein involved in intracellular signal pathways involved in regulating cellular adhesion, proliferation, migration and invasion); TOM1 (a negative regulator of the IL-1beta and TNF-alpha signaling pathways); CXCL12 (a chemokine, is regulated by miR-126); POU3F1 (a factor required for the activation of the transcription factor PU.1); VEGF-A (protein production is reduced as miR-126 binds to the 3′ untranslated region of the VEGF-A mRNA);
IRS-1 (inhibiting the cell cycle from progressing from G0/G1 into S phase); and
HOXA9 (miR-126 modulates HOXA9 expression in haematopoietic cells).

A nucleic acid sequence for the miR-126 miRNA is:

(SEQ ID NO: 1)
5′CAUUAUUACUUUUGGUACGCG-3′.

c. Aptamers

In some embodiments, the functional nucleic acids are aptamers. Aptamers are molecules that interact with a target molecule, preferably in a specific way. Typically, aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with Kd values from the target molecule of less than 10⁻¹² M. It is preferred that the aptamers bind the target molecule with a Kd less than 10⁻⁶, 10⁻⁸, 10⁻¹⁰, or 10⁻¹². Aptamers can bind the target molecule with a very high degree of specificity. For example, aptamers have been isolated that have greater than a 10,000-fold difference in binding affinities between the target molecule and another molecule that differ at only a single position on the molecule. It is preferred that the aptamer have a Kd with the target molecule at least 10, 100, 1000, 10,000, or 100,000-fold lower than the Kd with a background binding molecule. It is preferred when doing the comparison for a molecule such as a polypeptide, that the background molecule be a different polypeptide.

d. Ribozymes

The functional nucleic acids can be ribozymes. Ribozymes are nucleic acid molecules that are capable of catalyzing a chemical reaction, either intra-molecularly or inter-molecularly. It is preferred that the ribozymes catalyze intermolecular reactions. Different types of ribozymes that catalyze nuclease or nucleic acid polymerase-type reactions which are based on ribozymes found in natural systems, such as hammerhead ribozymes are described. Ribozymes that are not found in natural systems, but which have been engineered to catalyze specific reactions de novo are also described. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for targeting specific cleavage of nucleic acids because recognition of the target substrate is based on the target substrates sequence.

e. Triplex Forming Oligonucleotides

The functional nucleic acids can be triplex forming oligonucleotide molecules. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a Kd less than 10⁻⁶, 10⁻⁸, 10⁻¹⁰, or 10⁻¹².

f. External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

### D. Additional Agents to be Delivered

The dendrimer-small biologics complexes can be used to deliver one or more additional agents, particularly one or more active agents to prevent or treat one or more symptoms of the target diseases or disorders. Suitable therapeutic, diagnostic, and/or prophylactic agents can be a biomolecule, such as peptides, proteins, carbohydrates, nucleotides or oligonucleotides. The agent can be encapsulated within the dendrimers, dispersed within the dendrimers, and/or associated with the surface of the dendrimer, either covalently or non-covalently.

Dendrimers have the advantage that multiple therapeutic, prophylactic, and/or diagnostic agents can be delivered with the same dendrimers. One or more types of agents can be encapsulated, complexed, or conjugated to the dendrimer. In one embodiment, the dendrimers are complexed with or conjugated to two or more different classes of agents, providing simultaneous delivery with different or independent release kinetics at the target site. In another embodiment, the dendrimers are covalently linked to at least one detectable moiety and at least one class of agents. In a further embodiment, dendrimer complexes each carrying different classes of agents are administered simultaneously for a combination treatment.

### 1. Therapeutic and Prophylactic Agents

In some embodiments, the dendrimer-small biologics complexes include one or more additional therapeutic or prophylactic agents. Exemplary additional therapeutic or prophylactic agents include anti-inflammatory agents, chemotherapy agents, and anti-infective agents.

a. Anti-Inflammatory Agents

In some embodiments, the compositions include one or more anti-inflammatory agents. Anti-inflammatory agents reduce inflammation and include steroidal and non-steroidal drugs.

A preferred anti-inflammatory is an antioxidant drug including N-acetylcysteine. Preferred non-steroidal anti-inflammatory drugs (“NSAIDS”) include mefenamic acid, aspirin, Diflunisal, Salsalate, Ibuprofen, Naproxen, Fenoprofen, Ketoprofen, Deacketoprofen, Flurbiprofen, Oxaprozin, Loxoprofen, Indomethacin, Sulindac, Etodolac, Ketorolac, Diclofenac, Nabumetone, Piroxicam, Meloxicam, Tenoxicam, Droxicam, Lornoxicam, Isoxicam, Meclofenamic acid, Flufenamic acid, Tolfenamic acid, elecoxib, Rofecoxib, Valdecoxib, Parecoxib, Lumiracoxib, Etoricoxib, Firocoxib, Sulphonanilides, Nimesulide, Niflumic acid, and Licofelone.

Representative small molecules include steroids such as methyl prednisone, dexamethasone, non-steroidal anti-inflammatory agents including COX-2 inhibitors, corticosteroid anti-inflammatory agents, gold compound anti-inflammatory agents, immunosuppressive, anti-inflammatory and anti-angiogenic agents, anti-excitotoxic agents such as valproic acid, D-aminophosphonovalerate, D-aminophosphonoheptanoate, inhibitors of glutamate formation/release, such as baclofen, NMDA receptor antagonists, salicylate anti-inflammatory agents, ranibizumab, anti-VEGF agents, including aflibercept, and rapamycin. Other anti-inflammatory drugs include nonsteroidal drug such as indomethacin, aspirin, acetaminophen, diclofenac sodium and ibuprofen. The corticosteroids can be fluocinolone acetonide and methylprednisolone.

Exemplary immune-modulating drugs include cyclosporine, tacrolimus and rapamycin. In some embodiments, anti-inflammatory agents are biologic drugs that block the action of one or more immune cell types such as T cells, or block proteins in the immune system, such as tumor necrosis factor-alpha (TNF-alpha), interleukin 17-A, interleukins 12 and 23.

In some embodiments, the anti-inflammatory drug is a synthetic or natural anti-inflammatory low molecular weight protein. Antibodies specific to select immune components can be added to immunosuppressive therapy. In some embodiments, the anti-inflammatory drug is fragment of an anti-T cell antibody (e.g., anti-thymocyte globulin or Anti-lymphocyte globulin), anti-IL-2Rα receptor antibody (e.g., basiliximab or daclizumab), or anti-CD20 antibody (e.g., rituximab).

Many inflammatory diseases may be linked to pathologically elevated signaling via the receptor for lipopolysaccharide (LPS), toll-like receptor 4 (TLR4). There has thus been great interest in the discovery of TLR4 inhibitors as potential anti-inflammatory agents. Recently, the structure of TLR4 bound to the inhibitor E5564 was solved, enabling design and synthesis of new TLR4 inhibitors that target the E5564-binding domain. These are described in U.S. Pat. No. 8,889,101. As reported by Neal, et al., PLoS One. 2013; 8(6): e65779e, a similarity search algorithm used in conjunction with a limited screening approach of small molecule libraries identified compounds that bind to the E5564 site and inhibit TLR4. The lead compound, C34, is a 2-acetamidopyranoside (MW 389) with the formula C₁₇H₂₇NO₉, which inhibits TLR4 in enterocytes and macrophages in vitro, and reduces systemic inflammation in mouse models of endotoxemia and necrotizing enterocolitis. Thus, in some embodiments, the active agents are one or more TLR4 inhibitors. In preferred embodiments, the active agents are C34, and derivatives, analogues thereof.

In preferred embodiments, the one or more anti-inflammatory drugs are released from the dendrimeric nanoparticles after administration to a mammalian subject in an amount effective to inhibit inflammation for at least 1 day, 2 days, 3 days, 4 days, 5 days, 6 days, preferably at least a week, 2 weeks, or 3 weeks, more preferably at least a month, two months, three months, four months, five months, six months.

b. Chemotherapeutic Agents

Chemotherapeutic agents generally include pharmaceutically or therapeutically active compounds that work by interfering with DNA synthesis or function in cancer cells. Based on their chemical action at a cellular level, chemotherapeutic agents can be classified as cell-cycle specific agents (effective during certain phases of cell cycle) and cell-cycle nonspecific agents (effective during all phases of cell cycle). Examples of chemotherapeutic agents include alkylating agents, angiogenesis inhibitors, aromatase inhibitors, antimetabolites, anthracyclines, antitumor antibiotics, platinum drugs, topoisomerase inhibitors, radioactive isotopes, radiosensitizing agents, checkpoint inhibitors, PD1 inhibitors, plant alkaloids, glycolytic inhibitors and prodrugs thereof.

Examples of PD-1 inhibitors include, for example, MDX-1106 is a genetically engineered, fully human immunoglobulin G4 (IgG4) monoclonal antibody specific for human PD-1, and pembrolizumab, recently approved by the US FDA. Fragment may be conjugated to dendrimers.

Representative chemotherapeutic agents include, but are not limited to, amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, taxol and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

Dendrimer complexes including one or more chemotherapeutic agents can be used prior to, or in conjunction with an immunotherapy such inhibition of checkpoint proteins such as PD-1 or CTLA-4, adoptive T cell therapy, and/or a cancer vaccine. Methods of priming and activating T cells in vitro for adaptive T cell cancer therapy are known in the art. See, for example, Wang, et al, Blood, 109(11):4865-4872 (2007) and Hervas-Stubbs, et al, J. Immunol., 189(7):3299-310 (2012). Examples of cancer vaccine include, for example, PROVENGE® (sipuleucel-T), which is a dendritic cell-based vaccine for the treatment of prostate cancer (Ledford, et al., Nature, 519, 17-18 (5 Mar. 2015). Such vaccines and other compositions and methods for immunotherapy are reviewed in Palucka, et al., Nature Reviews Cancer, 12, 265-277 (April 2012).

In some embodiments, the dendrimer complexes are effective to treat, image, and/or prevent inflammation of the microglia of the brain in neurodevelopmental disorders, including, for example Rett syndrome. In a preferred embodiment, the dendrimer complex would be used to deliver an anti-inflammatory agent (D-NAC) and anti-excitotoxic and D-anti-glutamate agents. Preferred candidates are: MK801, Memantine, Ketamine, 1-MT.

c. Neuroactive Agents

A number of drugs have been developed and used in an attempt to interrupt, influence, or temporarily halt the glutamate excitotoxic cascade toward neuronal injury. One strategy is the “upstream” attempt to decrease glutamate release. This category of drugs includes riluzole, lamotrigine, and lifarizine, which are sodium channel blockers. The commonly used nimodipine is a voltage-dependent channel (L-type) blocker. Attempts have also been made to affect the various sites of the coupled glutamate receptor itself. Some of these drugs include felbamate, ifenprodil, magnesium, memantine, and nitroglycerin. These “downstream” drugs attempt to influence such intracellular events as free radical formation, nitric oxide formation, proteolysis, endonuclease activity, and ICE-like protease formation (an important component in the process leading to programmed cell death, or apoptosis).

Active agents for the treatment of neurodegenerative diseases are well known in the art and can vary based on the symptoms and disease to be treated. For example, conventional treatment for Parkinson's disease can include levodopa (usually combined with a dopa decarboxylase inhibitor or COMT inhibitor), a dopamine agonist, or an MAO-B inhibitor.

Treatment for Huntington's disease can include a dopamine blocker to help reduce abnormal behaviors and movements, or a drug such as amantadine and tetrabenazine to control movement, etc. Other drugs that help to reduce chorea include neuroleptics and benzodiazepines. Compounds such as amantadine or remacemide have shown preliminary positive results. Hypokinesia and rigidity, especially in juvenile cases, can be treated with antiparkinsonian drugs, and myoclonic hyperkinesia can be treated with valproic acid. Psychiatric symptoms can be treated with medications similar to those used in the general population. Selective serotonin reuptake inhibitors and mirtazapine have been recommended for depression, while atypical antipsychotic drugs are recommended for psychosis and behavioral problems.

Riluzole (RILUTEK®) (2-amino-6-(trifluoromethoxy) benzothiazole), an anti-excitotoxin, has yielded improved survival time in subjects with ALS. Other medications, most used off-label, and interventions can reduce symptoms due to ALS. Some treatments improve quality of life and a few appear to extend life. Common ALS-related therapies are reviewed in Gordon, Aging and Disease, 4(5):295-310 (2013), see, e.g., Table 1 therein. A number of other agents have been tested in one or more clinical trials with efficacies ranging from non-efficacious to promising. Exemplary agents are reviewed in Carlesi, et al., Archives Italiennes de Biologie, 149:151-167 (2011). For example, therapies may include an agent that reduces excitotoxicity such as talampanel (8-methyl-7H-1,3-dioxolo(2,3)benzodiazepine), a cephalosporin such as ceftriaxone, or memantine; an agent that reduces oxidative stress such as coenzyme Q10, manganoporphyrins, KNS-760704 [(6R)-4,5,6,7-tetrahydro-N6-propyl-2,6-benzothiazole-diamine dihydrochloride, RPPX], or edaravone (3-methyl-1-phenyl-2-pyrazolin-5-one, MCI-186); an agent that reduces apoptosis such as histone deacetylase (HDAC) inhibitors including valproic acid, TCH346 (Dibenzo(b,f)oxepin-10-ylmethyl-methylprop-2-ynylamine), minocycline, or tauroursodeoxycholic Acid (TUDCA); an agent that reduces neuroinflammation such as thalidomide and celastol; a neurotropic agent such as insulin-like growth factor 1 (IGF-1) or vascular endothelial growth factor (VEGF); a heat shock protein inducer such as arimoclomol; or an autophagy inducer such as rapamycin or lithium.

Treatment for Alzheimer's Disease can include, for example, an acetylcholinesterase inhibitor such as tacrine, rivastigmine, galantamine or donepezil; an NMDA receptor antagonist such as memantine; or an antipsychotic drug.

Treatment for Dementia with Lewy Bodies can include, for example, acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48(1):1-8 (2012)).

Exemplary neuroprotective agents are also known in the art in include, for example, glutamate antagonists, antioxidants, and NMDA receptor stimulants. Other neuroprotective agents and treatments include caspase inhibitors, trophic factors, anti-protein aggregation agents, therapeutic hypothermia, and erythropoietin.

Other common active agents for treating neurological dysfunction include amantadine and anticholinergics for treating motor symptoms, clozapine for treating psychosis, cholinesterase inhibitors for treating dementia, and modafinil for treating daytime sleepiness.

d. Anti-Infective Agents

Antibiotics include beta-lactams such as penicillin and ampicillin, cephalosporins such as cefuroxime, cefaclor, cephalexin, cephydroxil, cepfodoxime and proxetil, tetracycline antibiotics such as doxycycline and minocycline, macrolide antibiotics such as azithromycin, erythromycin, rapamycin and clarithromycin, fluoroquinolones such as ciprofloxacin, enrofloxacin, ofloxacin, gatifloxacin, levofloxacin and norfloxacin, tobramycin, colistin, or aztreonam as well as antibiotics which are known to possess anti-inflammatory activity, such as erythromycin, azithromycin, or clarithromycin.

### 2. Diagnostic Agents

Dendrimer nanoparticles can include diagnostic agents useful for determining the location of administered particles. These agents can also be used prophylactically. Exemplary diagnostic materials include paramagnetic molecules, fluorescent compounds, magnetic molecules, and radionuclides. Suitable diagnostic agents include, but are not limited to, x-ray imaging agents and contrast media. Radionuclides also can be used as imaging agents. Exemplary radioactive label include ¹⁴C, ³⁶Cl, ⁵⁷Co, ⁵⁸Co, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, ¹⁷⁵Yb. Examples of other suitable contrast agents include gases or gas emitting compounds, which are radioopaque. In some embodiments, the imaging agent to be incorporated into the dendrimer nanoparticles is a fluorophore (e.g., fluorescein isothiocyanate (FITC), phycoerythrin (PE)), an enzyme (e.g., alkaline phosphatase, horseradish peroxidase), element particles (e.g., gold particles).

In further embodiments, a singular dendrimer complex composition can simultaneously treat and/or diagnose a disease or a condition at one or more locations in the body.

### III. Pharmaceutical Formulations

Pharmaceutical compositions including dendrimers covalently conjugated with one or more small molecule biologics may be formulated in a conventional manner using one or more physiologically acceptable carriers including excipients and auxiliaries which facilitate processing of the active compounds into preparations which can be used pharmaceutically.

Proper formulation is dependent upon the route of administration chosen. In preferred embodiments, the compositions are formulated for parenteral delivery. In some embodiments, the compositions are formulated for intravenous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain one or more dendrimers covalently conjugated with one or more small molecule biologics in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

The compositions of dendrimers covalently conjugated with one or more small molecule biologics are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies can be used in formulating a range of dosages for human use.

In certain embodiments, the compositions of dendrimers covalently conjugated with one or more small molecule biologics are administered locally, for example, by injection directly into a site to be treated. In some embodiments, the compositions of dendrimers covalently conjugated with one or more small molecule biologics are injected, topically applied, or otherwise administered directly into the vasculature onto vascular tissue at or adjacent to a site of injury, surgery, or implantation. For example, in certain embodiments, the compositions of dendrimers covalently conjugated with one or more small molecule biologics are topically applied to vascular tissue that is exposed, during a surgical procedure. Typically, local administration causes an increased localized concentration of the compositions, which is greater than that which can be achieved by systemic administration.

Pharmaceutical compositions formulated for administration by parenteral (intramuscular, intraperitoneal, intravenous (IV) or subcutaneous injection) and enteral routes of administration are described.

### A. Parenteral Administration

In some embodiments, the compositions of dendrimers covalently conjugated with one or more small molecule biologics are administered parenterally.

The phrases “parenteral administration” and “administered parenterally” are art-recognized terms, and include modes of administration other than enteral and topical administration, such as injections, and include without limitation intravenous (i.v.), intramuscular (i.m.), intrapleural, intravascular, intrapericardial, intraarterial, intrathecal, intracapsular, intraorbital, intracardiac, intradermal, intraperitoneal (i.p.), transtracheal, subcutaneous (s.c.), subcuticular, intraarticular, subcapsular, subarachnoid, intraspinal and intrasternal injection and infusion. The dendrimers can be administered parenterally, for example, by subdural, intravenous, intrathecal, intraventricular, intraarterial, intra-amniotic, intraperitoneal, or subcutaneous routes.

For liquid formulations, pharmaceutically acceptable carriers may be, for example, aqueous or non-aqueous solutions, suspensions, emulsions or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

In some embodiments, the compositions of dendrimers covalently conjugated with one or more small molecule biologics are administered enterally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides and other components of infant formulas.

In preferred embodiments, the compositions of dendrimers covalently conjugated with one or more small molecule biologics are formulated for oral administration. Oral formulations may be in the form of chewing gum, gel strips, tablets, capsules or lozenges, and particles. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

### IV. Methods of Making Dendrimers and Nucleic Acid Conjugates Thereof

### A. Methods of Making Dendrimers

Dendrimers can be prepared via a variety of chemical reaction steps. Dendrimers are usually synthesized according to methods allowing controlling their structure at every stage of construction. The dendritic structures are mostly synthesized by two main different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward, building inward, and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis replies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of agents are linked to one type of dendron and a different type of agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in International Patent Publication Nos. WO2009/046446, WO2015168347, WO2016025745, WO2016025741, WO2019094952, and U.S. Pat. No. 8,889,101.

### B. Dendrimer Complexes

Dendrimer complexes can be formed of therapeutic, prophylactic or diagnostic small molecule biologics, such as functional nucleic acids, conjugated to a dendrimer, a dendritic polymer or a hyperbranched polymer. Conjugation of one or more agents to a dendrimer are known in the art and are described in detail in U.S. Published Application Nos. US 2011/0034422, US 2012/0003155, and US 2013/0136697.

In some embodiments, one or more agents are covalently attached to the dendrimers. In some embodiments, the agents are attached to the dendrimer via a linking moiety that is designed to be cleaved in vivo. The linking moiety can be designed to be cleaved hydrolytically, enzymatically, or combinations thereof, so as to provide for the sustained release of the agents in vivo. Both the composition of the linking moiety and its point of attachment to the agent, are selected so that cleavage of the linking moiety releases either an agent, or a suitable prodrug thereof. The composition of the linking moiety can also be selected in view of the desired release rate of the agents.

In some embodiments, the attachment occurs via one or more of disulfide, ester, ether, thioester, carbamate, carbonate, hydrazine, or amide linkages. In preferred embodiments, the attachment occurs via an appropriate spacer that provides an ester bond or an amide bond between the agent and the dendrimer depending on the desired release kinetics of the agent. In some cases, an ester bond is introduced for releasable form of agents. In other cases, an amide bond is introduced for non-releasable form of agents.

Linking moieties generally include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety is chosen in view of the desired release rate of the agents. In addition, the one or more organic functional groups can be selected to facilitate the covalent attachment of the agents to the dendrimers.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the agents in vivo. In embodiments where the linking moiety includes a spacer group, one or more organic functional groups will generally be used to connect the spacer group to both the nucleic acids and the dendrimers.

In preferred embodiments, the attachment can occur via an appropriate spacer that provides a disulfide bridge between the agent and the dendrimer. The dendrimer complexes are capable of rapid release of the agent in vivo by thiol exchange reactions, under the reduced conditions found in body. For example, spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP.

In some embodiments, the 5′ and 3′ ends of the sense or passenger strand and the 3′ end of the antisense strand are potential sites for conjugation with modifications. In preferred embodiments, the 5′ and/or 3′ ends of the sense or passenger strand are functionalized for conjugation to the dendrimers. In some embodiments, the hydroxyl surface group of the dendrimer is functionalized with a SPDP, optionally with a PEG linker for conjugation with nucleic acids.

In some embodiments, disulfide thiol-modifier is used for introducing sense 5′ thiol (—SH) linkage as shown in FIG. 2. In further embodiments, dithiol modified nucleic acid is treated with dithiothreitol (DTT) to quantitatively reduce diulfide bonds, resulting in sulfhydryl groups for further conjugation with dendrimer. In other embodiment, the sulfhydryl group in the sense 5′ end (e.g., siGFP) is then reacted with dendrimers functionalized with a SPDP, optionally with a PEG linker (e.g., dendrimer-PEG₄-SPDP), to form dendrimer and antisense molecule conjugate via a sulfhydryl exchange reaction.

Reactions and strategies useful for the covalent attachment of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given agent can be selected in view of the linking moiety desired, as well as the structure of the agents and dendrimers as a whole as it relates to compatibility of functional groups, protecting group strategies, and the presence of labile bonds.

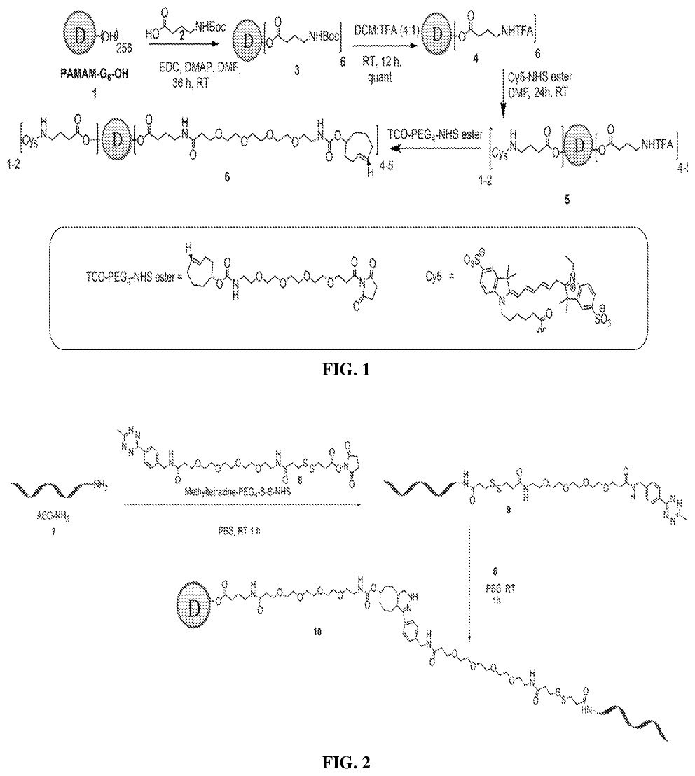

In some embodiments, the covalent attachment of functional nucleic acids to dendrimers occurs via click chemistry. In preferred embodiments, the covalent attachment of functional nucleic acids to dendrimers is via inverse electron demand Diels-Alder (IEDDA) reaction-initiated ligation between 1,2,4,5-tetrazines (Tz) and trans-cyclooctenes (TCO). In one embodiment, the antisense molecule is functionalized with terminal tetrazine (Tz) while hydroxyl terminated dendrimer is functionalized with trans-cyclooctene (TCO) for click reaction, for example, as shown in FIGS. 1 and 2.

The optimal drug loading will necessarily depend on many factors, including the choice of drug, dendrimer structure and size, and tissues to be treated. In some embodiments, the one or more agents are encapsulated, associated, and/or conjugated to the dendrimer at a concentration of about 0.01% to about 45%, preferably about 0.1% to about 30%, about 0.1% to about 20%, about 0.1% to about 10%, about 1% to about 10%, about 1% to about 5%, about 3% to about 20% by weight, and about 3% to about 10% by weight. However, optimal drug loading for any given drug, dendrimer, and site of target can be identified by routine methods, such as those described.

In some embodiments, conjugation of agents and/or linkers occurs through one or more surface and/or interior groups. Thus, in some embodiments, the conjugation of agents/linkers occurs via about 1%, 2%, 3%, 4%, or 5% of the total available surface functional groups, preferably hydroxyl groups, of the dendrimers prior to the conjugation. In other embodiments, the conjugation of agents/linkers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation. In preferred embodiments, dendrimer complexes retain an effective amount of surface functional groups for targeting to specific cell types, whilst conjugated to an effective amount of agents for treat, prevent, and/or image the disease or disorder.

### V. Methods of Use

Methods of using the dendrimer complex compositions are also described. In preferred embodiments, the dendrimer complexes cross impaired or damaged BBB and target activated microglia and astrocytes.

### A. Methods of Treatment

The compositions of dendrimers covalently conjugated with one or more small molecule biologics and formulations thereof can be administered to treat disorders associated with infection, inflammation, or cancer, particular those having systemic inflammation that extends to the nervous system, especially the CNS. The compositions can also be used for treatment of other diseases, disorders and injury including gastrointestinal disorders, proliferative diseases and treatment of other tissues where the nerves play a role in the disease or disorder. The compositions and methods are also suitable for prophylactic use.

The methods systemically administer one or more dendrimer-functional nucleic acid complexes in an amount effective to attenuate inflammatory cytokines and/or growth factors at a site in need thereof in a subject in need thereof. Typically, an effective amount of dendrimer complexes of dendrimers covalently conjugated with one or more small molecule biologics, optionally including one or more additional therapeutic, prophylactic, and/or diagnostic active agent are administered to an individual in need thereof. The dendrimers may also include a targeting agent, but as demonstrated by the examples, these are not required for delivery to activated macrophages, including those present at sites of injured tissue in the spinal cord and the brain.

In preferred embodiments, the dendrimer complexes include an agent that is attached or conjugated to dendrimers, which are capable of preferentially releasing the drug intracellularly under the reduced conditions found in vivo. The agent is covalently attached. The amount of dendrimer covalently conjugated with one or more small molecule biologics administered to the subject is selected to deliver an effective amount to reduce, prevent, or otherwise alleviate one or more clinical or molecular symptoms of the disease or disorder to be treated compared to a control, for example, a subject treated with the active agent without dendrimer.

### B. Conditions to be Treated

The compositions of dendrimers covalently conjugated with one or more small molecule biologics are suitable for treating one or more diseases, conditions, and injuries in the eye, the brain, and the nervous system, particularly those associated with pathological activation of microglia and astrocytes. The compositions can also be used for treatment of other diseases, disorders and injury including gastrointestinal disorders, cancer, and treatment of other tissues where the nerves play a role in the disease or disorder. The compositions and methods are also suitable for prophylactic use.

The dendrimer complex composition, preferably with a diameter under 15 nm and a hydroxyl group surface density at least 3 OH groups/nm², preferably under 10 nm and a hydroxyl group surface density of at least 4 OH groups/nm², more preferably under 5 nm and a hydroxyl group surface density of at least 5 OH groups/nm², and most preferably between 1-2 nm and a hydroxyl group surface density at least 4 OH groups/nm², delivering a therapeutic, prophylactic or diagnostic agent, selectively targets microglia and astrocytes, which play a key role in the pathogenesis of many disorders and conditions including neurodevelopmental, neurodegenerative diseases, necrotizing enterocolitis, and brain cancer. Thus, the dendrimer complexes are administered in a dosage unit amount effective to treat or alleviate conditions associated with the pathological conditions of microglia and astrocytes. Generally, by targeting these cells, the dendrimers deliver agent specifically to treat neuroinflammation.

### Microglia

Microglia are a type of neuroglia (glial cell) located throughout the brain and spinal cord. Microglia account for 10-15% of all cells found within the brain. As the resident macrophage cells, they act as the first and main form of active immune defense in the central nervous system (CNS). Microglia play a key role after CNS injury, and can have both protective and deleterious effects based on the timing and type of insult (Kreutzberg, G. W. Trends in Neurosciences, 19, 312 (1996); Watanabe, H., et al., Neuroscience Letters, 289, 53 (2000); Polazzi, E., et al., Glia, 36, 271 (2001); Mallard, C., et al., Pediatric Research, 75, 234 (2014); Faustino, J. V., et al., The Journal of Neuroscience: The Official Journal Of The Society For Neuroscience, 31, 12992 (2011); Tabas, I., et al., Science, 339, 166 (2013); and Aguzzi, A., et al., Science, 339, 156 (2013)). Changes in microglial function also affect normal neuronal development and synaptic pruning (Lawson, L. J., et al., Neuroscience, 39, 151 (1990); Giulian, D., et al., The Journal Of Neuroscience: The Official Journal Of The Society For Neuroscience, 13, 29 (1993); Cunningham, T. J., et al., The Journal of Neuroscience: The Official Journal Of The Society For Neuroscience, 18, 7047 (1998); Zietlow, R., et al., The European Journal Of Neuroscience, 11, 1657 (1999); and Paolicelli, R. C., et al., Science, 333, 1456 (2011)). Microglia undergo a pronounced change in morphology from ramified to an amoeboid structure and proliferate after injury. The resulting neuroinflammation disrupts the blood-brain-barrier at the injured site, and cause acute and chronic neuronal and oligodendrocyte death. Hence, targeting pro-inflammatory microglia should be a potent and effective therapeutic strategy. The impaired BBB in neuroinflammatory diseases can be exploited for transport of drug carrying nanoparticles into the brain.

In preferred embodiments, the dendrimers are administered in an amount effective to treat microglial-mediated pathology in the subject in need thereof without any associated toxicity.

In some embodiments, the subject to be treated is a human. In some embodiments, the subject to be treated is a child, or an infant. All the methods can include the step of identifying and selecting a subject in need of treatment, or a subject who would benefit from administration with the described compositions.

### 1. Ocular Diseases and Disorders

The compositions and methods are suitable for treatment of diseases, and disorders associated with the eye.

Examples of eye disorders that may be treated include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, meibomian gland dysfunction, anterior and posterior blepharitis, conjunctival hyperemia, conjunctival necrosis, cicatrical scaring and fibrosis, punctate epithelial keratopathy, filamentary keratitis, corneal erosions, thinning, ulcerations and perforations, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, anterior uveitis, posterior uveitis (including toxoplasmosis), pan-uveitis, inflammatory disease of the vitreous or retina, endophthalmitis prophylaxis and treatment, macular edema, macular degeneration, age-related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof. Other disorders include injury, burn, or abrasion of the cornea, cataracts and age-related degeneration of the eye or vision associated therewith.

In preferred embodiments, the eye disorder to be treated is associated with choroidal neovascularization (CNV). Exemplary eye disorders associated with CNV include macular degeneration. Therefore, in some embodiments, the methods deliver dendrimer conjugated functional nucleic acids to treat or prevent macular degeneration in a subject. In some embodiments the methods treat or prevent age-related (AMD).

Age-related macular degeneration (AMD) is a neurodegenerative, neuroinflammatory disease of the macula, which is responsible for central vision loss. The pathogenesis of age-related macular degeneration involves chronic neuroinflammation in the choroid (a blood vessel layer under the retina), the retinal pigment epithelium (RPE), a cell layer under the neurosensory retina, Bruch's membrane and the neurosensory retina, itself.

In some embodiments the methods administer OH-terminated dendrimers covalently conjugated to functional nucleic acids specific for suppression of angiogenesis and vascular integrity for the treatment or prevention of CNV in a subject in need thereof. Typically, the methods suppress CNV by about 10%-90%, for example, between 10% and 30%, selectively at sites of inflammation. Typically the methods systemically administer one or more dendrimer-functional nucleic acid complexes in an amount effective to attenuate VEGF production at a site in need thereof in a subject in need thereof. For example, in some embodiments, the methods reduce VEGF production by between about 10% and about 90% at a site in need thereof, for example, between 15% and 50%, between 20% and 30%, or 25%. In a particular embodiment the methods reduce VEGF levels by about ˜25% within the eye of a subject having or at risk of having macular degeneration associated with CNV in the eye.

### 2. Cancer

In some embodiments, the dendrimer compositions and formulations thereof are used in a method for treating a cancer in a subject in need of. The method for treating a cancer in a subject in need of including administering to the subject a therapeutically effective amount of the dendrimer compositions.

A cancer in a patient refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells. A tumor refers to all neoplastic cell growth and proliferation, whether malignant or benign, and all precancerous and cancerous cells and tissues. A solid tumor is an abnormal mass of tissue that generally does not contain cysts or liquid areas. A solid tumor may be in the brain, colon, breasts, prostate, liver, kidneys, lungs, esophagus, head and neck, ovaries, cervix, stomach, colon, rectum, bladder, uterus, testes, and pancreas, as non-limiting examples. In some embodiments, the solid tumor regresses or its growth is slowed or arrested after the solid tumor is treated with the presently disclosed methods. In other embodiments, the solid tumor is malignant. In some embodiments, the cancer comprises Stage 0 cancer. In some embodiments, the cancer comprises Stage I cancer. In some embodiments, the cancer comprises Stage II cancer. In some embodiments, the cancer comprises Stage III cancer. In some embodiments, the cancer comprises Stage IV cancer. In some embodiments, the cancer is refractory and/or metastatic. For example, the cancer may be refractory to treatment with radiotherapy, chemotherapy or monotreatment with immunotherapy. Cancer includes newly diagnosed or recurrent cancers, including without limitation, acute lymphoblastic leukemia, acute myelogenous leukemia, advanced soft tissue sarcoma, brain cancer, metastatic or aggressive breast cancer, breast carcinoma, bronchogenic carcinoma, choriocarcinoma, chronic myelocytic leukemia, colon carcinoma, colorectal carcinoma, Ewing's sarcoma, gastrointestinal tract carcinoma, glioma, glioblastoma multiforme, head and neck squamous cell carcinoma, hepatocellular carcinoma, Hodgkin's disease, intracranial ependymoblastoma, large bowel cancer, leukemia, liver cancer, lung carcinoma, Lewis lung carcinoma, lymphoma, malignant fibrous histiocytoma, a mammary tumor, melanoma, mesothelioma, neuroblastoma, osteosarcoma, ovarian cancer, pancreatic cancer, a pontine tumor, premenopausal breast cancer, prostate cancer, rhabdomyosarcoma, reticulum cell sarcoma, sarcoma, small cell lung cancer, a solid tumor, stomach cancer, testicular cancer, and uterine carcinoma. In some embodiments, the cancer is acute leukemia. In some embodiments, the cancer is acute lymphoblastic leukemia. In some embodiments, the cancer is acute myelogenous leukemia. In some embodiments, the cancer is advanced soft tissue sarcoma. In some embodiments, the cancer is a brain cancer. In some embodiments, the cancer is breast cancer (e.g., metastatic or aggressive breast cancer). In some embodiments, the cancer is breast carcinoma. In some embodiments, the cancer is bronchogenic carcinoma. In some embodiments, the cancer is choriocarcinoma. In some embodiments, the cancer is chronic myelocytic leukemia. In some embodiments, the cancer is a colon carcinoma (e.g., adenocarcinoma). In some embodiments, the cancer is colorectal cancer (e.g., colorectal carcinoma). In some embodiments, the cancer is Ewing's sarcoma. In some embodiments, the cancer is gastrointestinal tract carcinoma. In some embodiments, the cancer is a glioma. In some embodiments, the cancer is glioblastoma multiforme. In some embodiments, the cancer is head and neck squamous cell carcinoma. In some embodiments, the cancer is hepatocellular carcinoma. In some embodiments, the cancer is Hodgkin's disease. In some embodiments, the cancer is intracranial ependymoblastoma. In some embodiments, the cancer is large bowel cancer. In some embodiments, the cancer is leukemia. In some embodiments, the cancer is liver cancer. In some embodiments, the cancer is lung cancer (e.g., lung carcinoma). In some embodiments, the cancer is Lewis lung carcinoma. In some embodiments, the cancer is lymphoma. In some embodiments, the cancer is malignant fibrous histiocytoma. In some embodiments, the cancer comprises a mammary tumor. In some embodiments, the cancer is melanoma. In some embodiments, the cancer is mesothelioma. In some embodiments, the cancer is neuroblastoma. In some embodiments, the cancer is osteosarcoma. In some embodiments, the cancer is ovarian cancer. In some embodiments, the cancer is pancreatic cancer. In some embodiments, the cancer comprises a pontine tumor. In some embodiments, the cancer is premenopausal breast cancer. In some embodiments, the cancer is prostate cancer. In some embodiments, the cancer is rhabdomyosarcoma. In some embodiments, the cancer is reticulum cell sarcoma. In some embodiments, the cancer is sarcoma. In some embodiments, the cancer is small cell lung cancer. In some embodiments, the cancer comprises a solid tumor. In some embodiments, the cancer is stomach cancer. In some embodiments, the cancer is testicular cancer. In some embodiments, the cancer is uterine carcinoma.

a. Brain Tumors

Effective blood-brain tumor barrier (BBTB) penetration and uniform solid tumor distribution of the disclosed dendrimer can significantly enhance therapeutic delivery to brain tumors. High density hydroxyl surface groups with their small size, near neutral surface charge, selectively localize in cells associated with inflammation, particularly neuroinflammation.

The compositions and methods are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

The types of cancer that can be treated with the compositions and methods include, but are not limited to, brain tumors including glioma, glioblastoma, gliosarcoma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma, ganglioma, Schwannoma, cordomas and pituitary tumors.

The dendrimer complexes can be administered in combination with one or more additional therapeutically active agents, which are known to be capable of treating brain tumors or the symptoms associated therewith.

For example, the dendrimers may be administered to the brain via intravenous administration or during surgery to remove all or a part of the tumor. The dendrimers may be used to deliver chemotherapeutic agents, agents to enhance adjunct therapy such as of a subject undergoing radiation therapy, wherein the hydroxyl-terminated dendrimers are covalently linked to at least one radiosensitizing agent, in an amount effective to suppress or inhibit the activity of DDX3 in the proliferative disease in the brain.

It will be understood by those of ordinary skill in the art, that in addition to chemotherapy, surgical intervention and radiation therapy are also used in treatment of cancers of the nervous system. Radiation therapy means administering ionizing radiation to the subject in proximity to the location of the cancer in the subject. In some embodiments, the radiosensitizing agent is administered in two or more doses and subsequently, ionizing radiation is administered to the subject in proximity to the location of the cancer in the subject. In further embodiments, the administration of the radiosensitizing agent followed by the ionizing radiation can be repeated for 2 or more cycles.

Typically, the dose of ionizing radiation varies with the size and location of the tumor, but is dose is in the range of 0.1 Gy to about 30 Gy, preferably in a range of 5 Gy to about 25 Gy.

In some embodiments, the ionizing radiation is in the form of sterotactic ablative radiotherapy (SABR) or sterotactic body radiation therapy (SBRT).

### 3. Neurological and Neurodegenerative Diseases

The dendrimer compositions and formulations thereof can be used to diagnose and/or to treat one or more neurological and neurodegenerative diseases. The compositions and methods are particularly suited for treating one or more neurological, or neurodegenerative diseases associated with defective metabolism and functions of sphingolipids including sphingomyelin. In some embodiments, the disease or disorder is selected from, but not limited to, some psychiatric (e.g., depression, schizophrenia (SZ), alcohol use disorder, and morphine antinociceptive tolerance) and neurological (e.g., Alzheimer's disease (AD), Parkinson disease (PD)) disorders. In one embodiment, the dendrimer complexes are used to treat Alzheimer's Disease (AD) or dementia.

Neurodegenerative diseases are chronic progressive disorders of the nervous system that affect neurological and behavioral function and involve biochemical changes leading to distinct histopathologic and clinical syndromes (Hardy H, et al., Science. 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

Neuroinflammation, mediated by activated microglia and astrocytes, is a major hallmark of various neurological disorders making it a potential therapeutic target (Hagberg, H et al., Annals of Neurology 2012, 71, 444; Vargas, D L et al., Annals of Neurology 2005, 57, 67; and Pardo, C A et al., International Review of Psychiatry 2005, 17, 485). Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, M L et al., Nat Rev Neurosci 2007, 8, 57). The delivery of therapeutics across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can also be used to deliver active agents for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff's syndrome, post-encephalitic dementia, cancer and chemotherapy-associated cognitive impairment and dementia, and depression-induced dementia and pseudodementia.

In further embodiments, the disease or disorder is selected from, but not limited to, injection-localized amyloidosis, cerebral amyloid angiopathy, myopathy, neuropathy, brain trauma, frontotemporal dementia, Pick's disease, multiple sclerosis, prion disorders, diabetes mellitus type 2, fatal familial insomnia, cardiac arrhythmias, isolated atrial amyloidosis, atherosclerosis, rheumatoid arthritis, familial amyloid polyneuropathy, hereditary non-neuropathic systemic amyloidosis, Finnish amyloidosis, lattice corneal dystrophy, systemic AL amyloidosis, and Down syndrome. In preferred embodiments, the disease or disorder is Alzheimer's disease or dementia.

Criteria for assessing improvement in a particular neurological factor include methods of evaluating cognitive skills, motor skills, memory capacity or the like, as well as methods for assessing physical changes in selected areas of the central nervous system, such as magnetic resonance imaging (MRI) and computed tomography scans (CT) or other imaging methods. Such methods of evaluation are well known in the fields of medicine, neurology, psychology and the like, and can be appropriately selected to diagnosis the status of a particular neurological impairment. To assess a change in Alzheimer's disease, or related neurological changes, the selected assessment or evaluation test, or tests, are given prior to the start of administration of the dendrimer compositions. Following this initial assessment, treatment methods for the administration of the dendrimer compositions are initiated and continued for various time intervals. At a selected time-interval subsequent to the initial assessment of the neurological defect impairment, the same assessment or evaluation test (s) is again used to reassess changes or improvements in selected neurological criteria.

### C. Dosage and Effective Amounts

Dosage and dosing regimens are dependent on the severity and location of the disorder or injury and/or methods of administration, and is known to those skilled in the art. A therapeutically effective amount of the dendrimer composition used in the treatment of a neurological or neurodegenerative disease is typically sufficient to reduce or alleviate one or more symptoms of the neurological or neurodegenerative disease.

Preferably, the agents do not target or otherwise modulate the activity or quantity of healthy cells not within or associated with the diseased or target tissues, or do so at a reduced level compared to target cells including activated microglial cells in the CNS. In this way, by-products and other side effects associated with the compositions are reduced.

Administration of the compositions leads to an improvement, or enhancement, of neurological function in an individual with a neurological disease, neurological injury, or age-related neuronal decline or impairment. In some in vivo approaches, the dendrimer complexes are administered to a subject in a therapeutically effective amount to stimulate or induce neural mitosis leading to the generation of new neurons, providing a neurogenic effect. Also provided are effective amounts of the compositions to prevent, reduce, or terminate deterioration, impairment, or death of an individual's neurons, neurites and neural networks, providing a neuroprotective effect.

The actual effective amounts of dendrimer complex can vary according to factors including the specific agent administered, the particular composition formulated, the mode of administration, and the age, weight, condition of the subject being treated, as well as the route of administration and the disease or disorder. The dose of the compositions can be from about 0.01 to about 100 mg/kg body weight, from about 0.1 mg/kg to about 10 mg/kg, and from about 0.5 mg to about 5 mg/kg body weight. Generally, for intravenous injection or infusion, the dosage may be lower than for oral administration.

In general, the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, weekly, monthly or yearly dosing.

The compositions can be administered daily, biweekly, weekly, every two weeks or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

In some embodiments, the methods administer functional nucleic acids to a subject in an amount effective to reduce or prevent one or more diseases or disorders in the subject. Administering a functional nucleic acid to a subject as a dendrimer-functional nucleic acid complex typically enhances the serum half-life of the functional nucleic acid as compared to the serum half-life of the functional nucleic acid administered alone. In some embodiments conjugation with a dendrimer shields a functional nucleic acid from enzymic or proteolytic degradation, and prevents non-specific cellular uptake and/or activity of the functional nucleic acid. For example, in some embodiments, a functional nucleic acid has a serum half-life that is between 10% and 10,000% greater than that of the same functional nucleic acid in the absence of conjugation with a dendrimer, such as 50%, 100%, 150%, 200%, 250%, 300%, 350%, 400%, 500%, 700%, 1,000%, 5,000% or 10,000%, or more than 10,000% greater than that of the same functional nucleic acid in the absence of conjugation with a dendrimer. As set forth in the examples, a miRNA molecule having a serum half-life of 30 minutes in vivo was functional up to 14 days following administration as dendrimer conjugate. Therefore, in some embodiments, the functional nucleic acids generally provide therapeutic efficacy for a period of greater than 30 minutes following administration in vivo, for example, up to 1 hour (1 hr), 2 hrs, 3 hrs, 4 hrs, 5 hrs, 6 hrs, 7 hrs, 8 hrs, 9 hrs, 10, hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 2 days, 3 days, 4 days, 5 days, 6 days, one week, two weeks, three weeks, 4 weeks, one month or more than one month following administration in vivo.

In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

### D. Controls

The effect of dendrimer complex compositions can be compared to a control. Suitable controls are known in the art and include, for example, untreated cells or an untreated subject. In some embodiments, the control is untreated tissue from the subject that is treated, or from an untreated subject. Preferably the cells or tissue of the control are derived from the same tissue as the treated cells or tissue. In some embodiments, an untreated control subject suffers from, or is at risk from the same disease or condition as the treated subject.

### VI. Kits

The compositions can be packaged in kit. The kit can include a single dose or a plurality of doses of a composition including one or more functional nucleic acids encapsulated in, associated with, or conjugated to a dendrimer, and instructions for administering the compositions. Specifically, the instructions direct that an effective amount of the composition be administered to an individual with a particular disease or disorder as indicated. The composition can be formulated as described above with reference to a particular treatment method and can be packaged in any convenient manner.

The present invention will be further understood by reference to the following non-limiting examples.

### EXAMPLES

### Example 1: Synthesis and Characterization of Dendrimer-Antisense-RNA Conjugates Including Cy-5

### Materials and Methods

Unless stated otherwise, all the reactions were performed in flame-dried glassware under a positive pressure of nitrogen using dry solvents. After each reaction step, the products were purified via dialysis in DMF for 24 h to eliminate small molecule impurities followed by water dialysis to remove DMF. All recitations of chemical structures as (1)-(9) correspond with the chemical structures represented as (1)-(9) in FIGS. 1 and 2.

¹H NMR (in DMSO-d6) comparison of intermediates and the final conjugates from top to bottom confirmed the product formation by appearance and disappearance of peaks and showing shifts in the retention times, respectively. Molecular weights of all intermediates and final components were determined by analytical HPLC traces of PAMAM-G6-OH, Cy5-D, and Cy5-D-PEG₄-TCO, and matrix-assisted laser desorption/ionization-time of flight (MALDI-TOF) spectra of PAMAM-G6-OH, Cy5-D, and Cy5-D-PEG₄-TCO. The degree of conjugation in each step of the synthesis was calculated based on the 1H-NMR and change of molecular weights measured by MALDI-TOF/MS.

### Biomolecules, Chemicals, and Reagents

Commercial grade reagents and anhydrous solvents were purchased from chemical suppliers and used without further purification. 1-Ethyl-3-(3-dimethylaminopropyl)carbodiimide (EDC·HCl), N, N-diisoprpylethylamine (DIPEA), 4-(dimethylamino)pyridine (DMAP) trifluoracetic acid (TFA), γ-(Boc-amino)butyric acid (Boc-GABA-OH), anhydrous dichloromethane (DCM), N,N′-dimethylformamide (DMF) were purchased from Sigma-Aldrich (St. Louis, MO, USA). Cyanine 5 (Cy5)-mono-NHS ester was purchased from Amersham Bioscience-GE Healthcare. Trans-cyclooctene (TCO) was purchased from AAT bioquest, Inc. Tetrazine (Tz) precursors were purchased from BroadPharm. Deuterated solvents dimethylsulfoxide (DMSO-d6), water (D2O), and Chloroform (CDCl3) were purchased from Cambridge Isotope Laboratories Inc. (Andover, MA). Ethylenediamine-core polyamidoamine (PAMAM) dendrimer, generation 6.0, hydroxy surface (G6-OH; diagnostic grade; consisting of 256 hydroxyl end-groups), methanol solution (13.75% w/w) was purchased from Dendritech Inc. (Midland, MI, USA). Dialysis membranes were purchased from Spectrum Laboratories Inc. (Rancho Dominguez, CA, USA).

### Instrumentation for Characterization of Intermediates and Products

Proton nuclear magnetic resonance (1H NMR) spectra were recorded on a Bruker 500 MHz spectrometer at ambient temperatures and analyzed using MestReNova software. ¹H NMR chemical shifts were reported as δ using residual solvent as an internal standard (DMSO-d6, 2.50), and (D₂O, 4.79 ppm).

### Synthesis of D-GABABoc, (3)

A solution of PAMAM G6-OH 1 (1.00 g, 0.017 mmol) in DMF (12 mL) was treated with Boc-GABA-OH (0.069 g, 0.34 mmol), DMAP (0.0782 g, 0.408 mmol) and stirred at room temperature for 5 min. Then EDC·HCl (0.046 g, 0.374 mmol) was added in portions to the reaction mixture over the period of 5 min. The reaction mixture was stirred at room temperature for 36 h. The crude product was transferred to 3 kD MW cut-off cellulose dialysis tubing and dialysed against DMF 12 h followed by water for 24 h. The aqueous layer was frozen and lyophilized to yield desired product 3 as a hygroscopic white solid (0.973 g, 95%). ¹H NMR (500 MHz, DMSO-d6) 8.10-7.70 (m, internal amide H), 6.60 (s, GABA amide H, 10H), 4.74 (s, surface OH, 213H), 3.99 (s, ester linked H, 22H) 3.39 (t, J=5.0 Hz, dendrimer —CH₂), 3.40-3.35 (m, dendrimer CH₂), 3.11 (m, dendrimer-CH₂), 2.89 (m, dendrimer CH₂), 2.73-2.65 (m, dendrimer CH₂), 2.45 (m, dendrimer-CH₂), 2.21 (m, dendrimer CH₂), 1.64-1.59 (m, GABA linker-CH₂, 25H), 1.36 (s, Boc group, 85H). HPLC C18 retention time 19 min.

### Synthesis of D-GABA-NH2, (4)

The Boc protected GABA linker containing PAMAM G6-OH 3 (250 mg, 0.004 mmol) was treated with TFA/DCM (3:4) solvent mixture. The reaction was stirred at room temperature for 12 h, then diluted with methanol, and concentrated in vacuo (this step is necessary to remove excess TFA and hydrolytic cleavage of GABA linker). The crude product was used for the next step without any further purification. 1H NMR (500 MHz, DMSO-d6) δ 8.50-7.75 (m, internal amide H), 5.50-4.50 (broad s, surface —OH), 4.00 (s, ester linked H), 3.50-2.25 (m, dendrimer-CH₂), 1.93-1.59 (m, GABA linker-CH₂).

### Synthesis of Cy5-D, (5)

A solution of compound 4 (287 mg, 0.0048 mmol) in DMF (5 mL) was treated with DIPEA to adjust pH of the reaction mixture (˜7.0-7.5). Then reaction was treated with Cy5-NHS ester (8.7 mg, 0.0115 mmol, 1.2 eq) and stirred at rt for 12 h. It was then dialysed against DMF for 12 h followed by against water for 24 h. The aqueous layer was frozen and lyophilized to yield desired product 5 as blue solid (yield 85%). 1H NMR (500 MHz, DMSO-d6) δ 8.25-7.75 (m, internal amide H), 7.30 (s, Cy5 H), 7.10 (s Cy5 H), 6.70 (s, GABA amide H), 6.50 (m Cy5 H), 6.25 (m Cy5 H), 4.75 (s, surface OH, 226H), 4.00 (m, ester CH₂), 3.50-2.00 (m, dendrimer CH₂), 1.64-1.59 (s, 31H), 1.25 (s, 66H), 0.8 (s, 21H). HPLC C18 retention time (Acetronitrile in H₂O with 0.1% TFA, linear gradient, 40 min). HPLC C18 retention time: 17.5 min.

### Synthesis of Cy5-D-PEG₄-TCO, (6)

A solution of compound 5 (48 mg, 0.0008 mmol) in DMF (5 mL) was treated with DIPEA to adjust pH of the reaction mixture (˜7.0-7.5). The reaction was treated with TCO-PEG₄-NHS ester (4 mg, 0.0080 mmol) and stirred the reaction mixture at rt for 12 h. It was then dialysed against DMF 12 h followed by against water for 24 h. The aqueous layer was frozen and lyophilized to yield desired product as blue solid (yield 55%). 1H NMR (500 MHz, DMSO-d6) δ 8.14-7.73 (m, internal amide H), 7.35 (m, Cy5 H), 7.25 (m, Cy5 H), 7.05 (m, Cy5 H), 6.6 (m, Cy5 H), 6.3 (m, Cy5 H), 6.83 (s, GABA amide H), 5.65-5.50 (m, TCO H), 5.45-5.35 (m, TCO H), (4.74 (s, surface OH, H), 4.01-3.39 (t, J=5.0 Hz, ester —CH₂), 3.50-2.00 (m, dendrimer CH₂), 1.9 (s, 24H), 1.6 (s, 80H), 1.2 (s, 126H), 0.8 (s, 80H). HPLC C18 retention time: 19.5 min.

### Ultrafiltration and SEC Chromatography

After each step of the synthesis, the excess small molecules reagents and byproducts and buffer exchange were performed by Amicon ultrafiltration using 15 mL, 10 kDa and 30 kDa MWCO units (for ≥2 mg samples) or 0.5 mL, 10 kDa and 30 kDa MWCO units (for ≤2 mg samples).

### MALDI-TOF of PAMAM Dendrimer Conjugates

The MALDI matrix 2′,4′,6′-Trihydroxyacetophenone monohydrate (THAP) (10 mg) was dissolved in 1 mL of Acetonitrile in water (1:1) with 0.1% trifluoroacetic acid). Then 2 μL of PAMAM dendrimer was deposited on the MALDI sample plate. The matrix (2 μL of the 10 mg/mL) was deposited on the air-dried sample and allowed it to air dry for 10-20 min. The MALDI-TOF MS analysis was performed in a reflective-positive mode.

### Results

### Synthesis and Characterization of Cy5-D-PEG₄-TCO

Cy5-D-PEG₄-TCO conjugate was synthesized using PAMAM-G6-OH (D6-OH) dendrimer containing 256 free hydroxyl groups (D6-OH) available on the surface for further conjugations. The D6-OH in methanol (13.75% w/w) was dried under reduced pressure, followed by the dissolution in water and lyophilization for further conjugation. The lyophilized mono-functionalized D6-OH was functionalized with Boc protected amine by treatment of 4-tert-butoxycarbonylamino)butyric acid (Boc-GABA-OH) under N-(3-dimethylaminopropyl)-N′-ethylcarbodiimide hydrochloride (EDC·HCl) and 4-(dimethylamino)pyridine (4-DMAP) in DMF for 36 h at room temperature to yield the Boc protected bifunctional dendrimer product. The crude dendrimer was dialyzed by 3.5 kDa membrane against ultrapure water for 24 h followed by lyophilization. ¹H NMR of dendrimer (3) depicted the appearance of tert-butyl protons of Boc group at δ 1.3 ppm as a singlet along with GABA methylene protons at δ 1.6 ppm. The peak at δ 3.9 ppm is for the methylene protons of the dendrimer next to hydroxyl groups once converted to ester and amidic protons from GABA linker also appeared at δ 6.8 ppm. Then the Boc groups were de-protected under mild acidic condition using trifluoroacetic acid (TFA) in dichloromethane (DCM) 1:4 to obtain bi-functionalized dendrimer. The excess TFA was removed by co-evaporation with methanol and resulted crude product was used for next step without further purification. The complete disappearance of Boc protons was confirmed by ¹H NMR while no ester hydrolysis was observed under this condition (1H NMR (DMSO-d6, 500 MHz) characterization of dendrimer conjugates, D-GABA-Boc, D-GABA-NH₂, Cy5-D, Cy5-D-PEG₄-TCO (in DMSO-d6 and D₂O) showed the appearance or disappearance of characteristic signals. The total number of amine groups was maintained at ˜10. Then bifunctional dendrimer was treated with fluorescent dye Cy5 to yield dendrimer (4) with ˜1-2 successful Cy5 attachment at dendrimer surface. ¹H NMR showed the appearance of Cy5 signals in the aromatic region and HPLC retention time was shifted from 19.0 to 17.5 min confirming the product formation. After Cy5 attachment rest of amine groups were reacted with trans-cyclooctene containing, hetero-bi functional (NHS-PEG₄-TCO) linker. This hetero-bi functional linker used to form a chemical bonding between dendrimer and Antisense oligonucleotide (ASO).

### Synthesis and Characterization of Dendrimer-ASO Conjugates

The ASO was functionalized with terminal tetrazine (Tz) while D6-OH was functionalized with trans-cyclooctene (TCO) for click reaction (FIGS. 1-2). ASO (2 mg in 500 μL of PBS) was treated with Methyl tetrazine-PEG4-S—S—NHS ester (5 mol equiv in 10-20 μL of anhydrous DMSO) and incubated for 1 h. The excess Me-Tz-PEG₄-S—S—NHS and byproducts were removed by ultrafiltration. The TCO-PEG₄ attached dendrimer 6 (17 mg in 500 μL PBS) was reacted with 8 via trans-cyclooctene-tetrazine (TCO-Tz) to afford crude product 9. The resulting crude product was purified by ultrafiltration, and the product was further purified by GE Healthcare SEPHADEX® G-25 column and concentrated by ultrafiltration. The molecular weight was determined by MALDI-TOF (MALDI-TOF spectrum of Cy5-D-ASO showed a peak at mass of 66009 Da for D-ASO; a gel retardation assay was performed to confirm the formation of the D-ASO conjugate, whereby RNA ladder (NEB, Ipswich, MA), free siRNA, and D-siGFP were mixed with GELRED® stain, 1 μL of glycerol, and ultrapure water for a nucleic acid loading of 2 μg; Gel electrophoresis was performed in 3% TBE-Urea gel with TBE buffer (Bio-Rad, Hercules, CA) at 120 V for 20 min, after which the gel was imaged in a CHEMIDOC® Imaging System (Bio-Rad, Hercules)). Further, the successful synthesis of D-siGFP was confirmed by gel electrophorosis. The TCO-Tz click reaction used herein is fast, quantitative and releases no toxic byproducts. At low biomolecule concentrations (less than <5 μM) TCO-Tz works well compared to strain-promoted alkyne-azide cycloaddition SPAAC and Cu(I) catalyzed azide-alkyne cycloaddition (CuAcc). The TCO-Tz “click” reaction proceeds via an inverse-electron demand Diels-Alder reaction (IEDDA) between a TCO and a Tz, followed by retro Diels-Alder reaction eliminating N2 to form a dihydropyridazine bond. In contrast to a regular Diels-Alder reaction, where an electron diene reacts with an electron-poor dienophile, in an inverse-electron-demand Diels-Alder reaction, an electron-rich dienophile reacts with an electron poor diene. The TCO as a precursor gave tremendous rate difference compared to cis-cyclooctene and other cyclic alkenes. The high reactivity is related to a crown confirmation adapted by TCO, which is lower in energy than the ‘half-chair’ confirmation of cis form. The chemoselective TCO-Tz ligation possess ultrafast kinetics (>800 M⁻¹s⁻¹) unmatched by any other bio-orthogonal ligation pair. The click ligation was performed at near neutral pH, aqueous condition at room temperature. The ultrafast kinetics, selectivity, and long-term aqueous stability make TCO-Tz the ideal pair in low concentration dendrimer-ASO coupling reactions.

### Example 2: Development of a Hydroxyl PAMAM Dendrimer- and siRNA-based Nano-Conjugate as a Targeted Therapeutic for CNS Disorders

### Materials and Methods

### Biomolecules, Chemicals, and Reagents

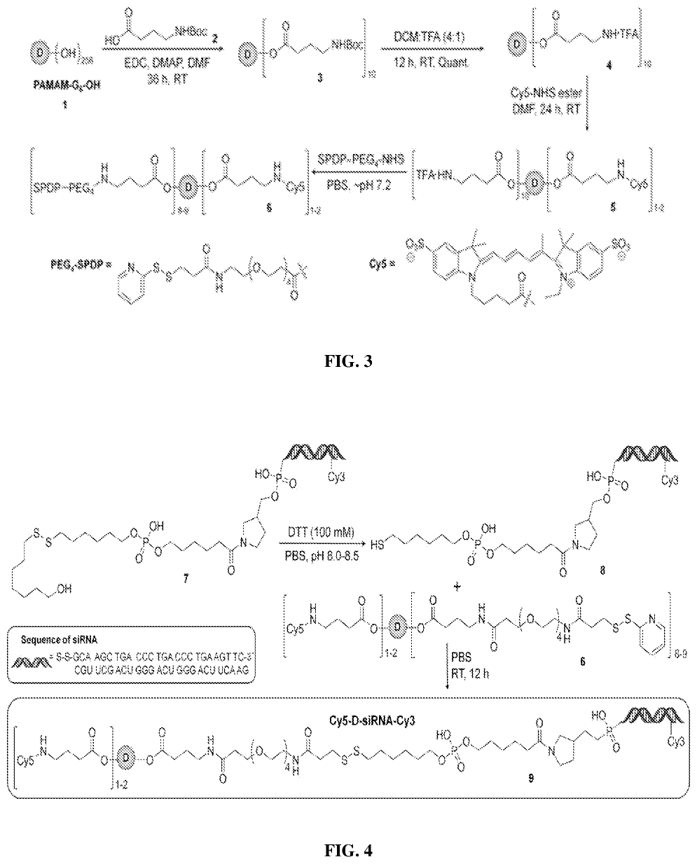

Unless stated otherwise, reactions were performed in flame dried glassware under a positive pressure of nitrogen using dry solvents. All recitations of chemical structures as (1)-(9) correspond with the chemical structures represented as (1)-(9) in FIGS. 3 and 4.

Commercial grade reagents and anhydrous solvents were purchased from chemical suppliers and used without further purification. 1-Ethyl-3-(3-dimethylaminopropyl)carbodiimide (EDC·HCl), N, N-diisopropylethylamine (DIPEA), 4-(dimethylamino)pyridine (DMAP) trifluoracetic acid (TFA), γ-(Boc-amino)butyric acid (Boc-GABA-OH), anhydrous dichloromethane (DCM), N,N′-dimethylformamide (DMF) were purchased from Sigma-Aldrich (St. Louis, MO, USA). Cyanine 5 (Cy5)-mono-NHS ester was purchased from Amersham Bioscience-GE Healthcare. Deuterated solvents dimethylsulfoxide (DMSO-d6), water (D₂O), and Chloroform (CDCl3) were purchased from Cambridge Isotope Laboratories Inc. (Andover, MA). Ethylenediamine-core polyamidoamine (PAMAM) dendrimer, generation 6.0, hydroxy surface (G6-OH; diagnostic grade; consisting of 256 hydroxyl end-groups), methanol solution (13.75% w/w) was purchased from Dendritech Inc. (Midland, MI, USA). Dialysis membranes were purchased from Spectrum Laboratories Inc. (Rancho Dominguez, CA, USA). GFP siRNA targeting sequence 5′-S—S-GCA AGC TGA CCC TGA CCC TGA AGT TC-3′ (SEQ ID NO: 2), GFP siRNA Cy3 5′-S—S-GCA AGC TGA CCC TGA CCC TGA AGT TC-Cy3-3′ (SEQ ID NO: 3), and scrambled RNA (scRNA) were purchased from Dharmacon (Lafayette, CO). Dulbecco's modified Eagle medium (DMEM, low glucose with L-glutamine), LIPOFECTAMINE® 2000, and streptomycin (10 mg/mL) were purchased from Life Technologies. All primers were purchased from IDT. RNase III was purchased from Thermo Scientific (Rockford, IL, USA). Magnesium chloride (MgCl2) and 1,4-dithiothreitol (DTT) were purchased from Sigma-Aldrich (St Louis, MO, USA).

### Instrumentation

Proton nuclear magnetic resonance (1H NMR) spectra were recorded on a Bruker 500 MHz spectrometer at ambient temperatures and analyzed using software. 1H NMR chemical shifts were reported as δ using residual solvent as an internal standard (DMSO-d6, 2.50), and (D₂O, 4.79 ppm). Analytical high-performance liquid chromatography (HPLC) was performed using a Shimadzu LC-AD HPLC system equipped with a variable wavelength absorbance detector and a C18 reverse phase column (Waters, BEH300 5 μm, 19×250 mm). The eluents were monitored at 210 nm using a photodiode array (PDA) detector, and fluorescently labeled conjugate was monitored at both 650 and 210 nm using fluorescence and PDI detectors respectively. HPLC elution was carried out with a 40 min linear gradient of 0%-90% HPLC grade acetonitrile (CH₃CN) in water (containing 0.1% TFA) maintaining the flow rate at 1.0 m/min.

### Synthesis of D-GABABoc, (3)

A solution of PAMAM G6-OH 1 (1.00 g, 0.017 mmol) in DMF (12 mL) was treated with Boc-GABA-OH (0.069 g, 0.34 mmol), DMAP (0.0782 g, 0.408 mmol) and stirred at room temperature for 5 min. Then EDC·HCl (0.046 g, 0.374 mmol) was added in portions to the reaction mixture over the period of 5 min. The reaction mixture was stirred at room temperature for 36 h. The crude product was transferred to 3 kD MW cut-off cellulose dialysis tubing and dialysed against DMF 12 h followed by water for 24 h. The aqueous layer was frozen and lyophilized to yield desired product 3 as a hygroscopic white solid (0.973 g, 95%). 1H NMR (500 MHz, DMSO-d6) 8.10-7.70 (m, internal amide H), 6.60 (s, GABA amide H, 10H), 4.74 (s, surface OH, 213H), 3.99 (s, ester linked H, 22H) 3.39 (t, J=5.0 Hz, dendrimer —CH₂), 3.40-3.35 (m, dendrimer CH₂), 3.11 (m, dendrimer-CH₂), 2.89 (m, dendrimer CH₂), 2.73-2.65 (m, dendrimer CH₂), 2.45 (m, dendrimer-CH₂), 2.21 (m, dendrimer CH₂), 1.64-1.59 (m, GABA linker-CH₂, 25H), 1.36 (s, Boc group, 85H). HPLC C18 retention time 19 min.

### Synthesis of D-GABA-NH2, (4)

The Boc protected GABA linker containing PAMAM G6-OH 3 (250 mg, 0.004 mmol) was treated with TFA/DCM (3:4) solvent mixture. The reaction was stirred at room temperature for 12 h, then diluted with methanol, and concentrated in vacuo (this step is necessary to remove excess TFA and hydrolytic cleavage of GABA linker). The crude product was used for the next step without any further purification. 1H NMR (500 MHz, DMSO-d6) δ 8.50-7.75 (m, internal amide H), 5.50-4.50 (broad s, surface —OH), 4.00 (s, ester linked H), 3.50-2.25 (m, dendrimer-CH₂), 1.93-1.59 (m, GABA linker-CH₂).

### Synthesis of Cy5-D, (5)

A solution of compound 4 (287 mg, 0.0048 mmol) in DMF (5 mL) was treated with DIPEA to adjust pH of the reaction mixture (˜7.0-7.5). Then reaction was treated with Cy5-NHS ester (8.7 mg, 0.0115 mmol, 1.2 eq) and stirred at rt for 12 h. It was then dialysed against DMF for 12 h followed by against water for 24 h. The aqueous layer was frozen and lyophilized to yield desired product 5 as blue solid (yield 85%). 1H NMR (500 MHz, DMSO-d6) δ 8.25-7.75 (m, internal amide H), 7.30 (s, Cy5 H), 7.10 (s Cy5 H), 6.70 (s, GABA amide H), 6.50 (m Cy5 H), 6.25 (m Cy5 H), 4.75 (s, surface OH, 226H), 4.00 (m, ester CH2), 3.50-2.00 (m, dendrimer CH₂), 1.64-1.59 (s, 31H), 1.25 (s, 66H), 0.8 (s, 21H). HPLC C18 retention time (Acetronitrile in H₂O with 0.1% TFA, linear gradient, 40 min). HPLC C18 retention time: 17.5 min.

### Synthesis of Cy5-D-PEG₄-SPDP, (6)

A solution of compound 5 (250 mg, 0.0041 mmol) in DMF (5 mL) was treated with DIPEA to adjust pH of the reaction mixture (˜7.0-7.5). The reaction was treated with SPDP-PEG₄-NHS ester (11 mg, 0.0020 mmol) and stirred the reaction mixture at rt for 12 h. It was then dialysed against DMF 12 h followed by against water for 24 h. The aqueous layer was frozen and lyophilized to yield desired product as blue solid (yield 80%). 1H NMR (500 MHz, DMSO-d6) δ 8.25-7.75 (m, internal amide H), 7.35 (m, Cy5 H), 7.25 (m, Cy5 H), 7.05 (m, Cy5 H), 6.6 (m, Cy5 H), 6.3 (m, Cy5 H), 6.83 (s, GABA amide H), 4.74 (s, surface OH, H), 4.01-3.39 (t, J=5.0 Hz, ester —CH2), 3.50-2.00 (m, dendrimer CH2), 1.9 (s, 24H), 1.6 (s, 80H), 1.2 (s, 126H), 0.8 (s, 80H). HPLC C18 retention time: 19.5 min.

Reduction of Thiol Modified siRNA

100 mM solution of DTT in 100 mM sodium phosphate buffer, pH 8.3-8.5 was prepared by dissolving 77.13 mg of DTT in 5 mL buffer. Thiol-modified siRNA 7 was dissolved in 125 μL of DTT solution and incubated at room temperature for 1 h. Byproduct removal was done using GE Healthcare NAP-10 columns SEPHADEX® G-25 DNA grade (CAS No. 2682-20-4). The NAP-10 column was equilibrated with ˜15 mL of 100 mM sodium Phosphate buffer, pH 6.0. The thiol-modified siRNA was eluted to Amicon Ultra-centrifuge, ULTRACEL® 10K filters (UFC501024) with 0.5 mL sodium phosphate buffer.

### Synthesis of D-siRNA Conjugate

A solution of compound 6 (2.4 mg, 37.5 nmol, 0.5 eq) in 200 μL was treated with siGFP-SH 8 (75 nmol, 1.0 eq) in 200 μL and stirred the reaction mixture at rt. After 12 h the mixture was passed through a GE Healthcare SEPHADEX® G-25 column and D-siGFP 9 product was collected. The product was concentrated and buffer exchange to PBS by centrifuged ultrafiltration using a 0.5 mL capacity 30 KDa MWCO filter unit. HPLC C18 retention time 18.5 min.

### Ultrafiltration and SEC Chromatography

The removal of excess reagents and byproducts after each step of the synthesis and buffer exchange were performed by ultracentrifugal filtration using 0.5 mL AMICON® filtration units with MWCO 30 kDa or 100 kDa. The Products and intermediated were further purified by Size-exclusion column (SEC) chromatography using PBS as the mobile phase.

### PAMAM Dendrimer Conjugates

The MALDI matrix 2-4′6′-Trihydroxyacetophenone monohydrate (THAP) (10 mg) was dissolved in 1 mL of Acetonitrile in water (1:1) with 0.1% trifluoroacetic acid). Then 2 μL of PAMAM dendrimer was deposited on the MALDI sample plate. The matrix (2 μL of the 10 mg/mL) was deposited on the air-dried sample and allowed it to air dry for 10-20 min. The MALDI-TOF MS analysis was performed in a reflective-positive mode.

### Oligonucleotide Conjugates

Matrix containing 3-hydroxypicolinic acid (3-HPA) and di-ammonium hydrogen citrate (DAHC) was used for oligonucleotide analysis. A solution of 3-HPA (50 mg/mL in 50% Acetronitrile/water) was mixed with DAHC solution (100 mg/mL) in 9:1 ratio (225 μL of 3-HPA:25 μL DAHC) to give final DAHC concentration 10 mg/mL. siRNA solution was desalted prior to mixing with matrix and 2 μL of siRNA was deposited on the plate and allowed it to air dry for 10-20 min. Then HPA/DAHC matrix was deposited on the air-dried oligonucleotide and allowed it to air dry. The MALDI-TOF MS analysis was performed on a Bruker Voyager DE-STR MALDI-TOF (Mass Spectrometric and Proteomics core, Johns Hopkins University, School of Medicine) operated in linear, positive ion mode.

### Gel Electrophoresis

A gel retardation assay was performed to confirm the formation of the D-siRNA conjugate. RNA ladder (NEB, Ipswich, MA), free siRNA, and D-siGFP were mixed with GelRed stain, 1 μL of glycerol, and ultrapure water for a nucleic acid loading of 2 μg. Gel electrophoresis was performed in 10% TBE-Urea gel with TBE buffer (Bio-Rad, Hercules, CA) at 120 V for 20 min, after which the gel was imaged in a ChemiDoc Imaging System (Bio-Rad, Hercules, CA). A separate retardation assay to visualize dendrimer was performed with 4-15% TGX stain-free gel (Bio-Rad,

### Serum Stability of Dendrimer-siRNA Conjugates

RNaseIII was used according to the manufacturer protocol for stability studies under reducing conditions. Studies with non-reducing conditions were performed with RNaseIII obtained through solvent exchange. 100 units of RNase III was diluted with equal volume of reaction buffer without DTT (50 mM NaCl, 10 mM Tris-HCl, 10 mM MgCl₂) and extracted with 10 kDa centrifugal filters. The process was repeated three times to ensure complete removal of residual DTT.

In reducing and non-reducing stability studies, 10 μg of free siGFP and D-siGFP were treated with 20 units of RNaseIII and stored in 37° C. Samples were taken at set timepoints, frozen immediately, and stored in −20° C. until further analysis. Gel retardation assay was performed in 10% TBE-Urea gel to determine RNA stability.

### Cell Line

The GFPd2 expressing human embryonic kidney 293T (HEK293T) cell line was generously provided by the Green Lab (Institute for NanoBio Technology, and Translational Tissue Engineering Center, Johns Hopkins University). Cells were cultured in Dulbecco's modified Eagle's medium (DMEM, ATCC, Manassas, VA) supplemented with 10% (v/v) heat-inactivated fetal bovine serum (FBS, Invitrogen Corp., Carlsbad, CA), 1% penicillin/streptomycin (P/S, Invitrogen Corp., Carlsbad CA). Cell media was replaced with Opti-MEM (Thermo Scientific, Rockford, IL) for transfection studies. Cells were maintained at 37° C. and 5% CO₂ under humidified atmosphere.

GL261 murine glioma cell line used for in vivo tumor inoculations were cultured in RPMI 1640 medium (Thermo Scientific, Rockford, IL) supplemented with 10% heat-inactivated fetal bovine serum, 1% penicillin/streptomycin, and 1% L-glutamine (Sigma-Aldrich, St. Louis, MO).

### In Vitro Evaluation of the Delivery Strategy

### Study of Time Dependent Uptake

GFP expressing HEK-293T cells were seeded in glass-bottom culture dishes and grown for 24-48 h to 70-80% confluency. Cells were treated with Cy5 fluorescently labeled dendrimer (Cy5-D) and siGFP-conjugated Cy5-labaled dendrimer (Cy5-D-siGFP, 9) in DMEM supplemented with 1% P/S (serum free media). The cells were then washed with PBS (×3) and fixed in 5% formalin solution. Cells were incubated and confocal microscopic images were taken by ZEISS AXIOVERT® 200 system equipped with an LSM 510-Meta confocal module. Image acquisition parameters were kept constant during the imaging. Images were processed by Zen 2011 software (Zeiss).

### Image Analysis

Live-cell images were taken with a ZEISS AXIOVERT® 200 phase-contrast microscope (Carl Zeiss) at set time points. Threshold for the images were automated with the built-in Triangle method in ImageJ and the threshold objects were used to calculate mean fluorescence and background. Automated cell counting was performed using “Analyze Particles” function on threshold masks and cell confluency was estimated with PHANTAST-FIJI® plug-in.

### HEK 293T Cell Transfection

Cells were seeded in 24-well tissue culture plates at density of 5×10⁴ cells per well and allowed to grow for 24 h. Different RNA delivery platforms (LIPOFECTAMINE® 2000, LIPOFECTAMINE® 3000, RNAi Max) were prepared following manufacturer protocol. Prior to treatment, cell culture medium was replaced with Opti-MEM reduced serum media. To optimize conditions for each delivery vehicle, combinations of siGFP payloads (0.6-30 pMol) and LIPOFECTAMINE® or RNAi Max concentrations (0.5-1.5 μL) were tested. Sets with the highest knockdown were used for comparison to the dendrimer platform.

For dose-dependent studies, cells were treated with different concentrations of D-siGFP over 48 h. Knockdown of GFPd2 fluorescence was assessed via live cell images taken at 0 h, 24 h, and 48 h post-treatment. Cellular proteins were also extracted at 48 h and stored at −80° C. for Western blotting.

### Western Blotting Assay of HEK293T Cells

Concentration of cellular protein was determined with BCA protein assay kit (Thermo Scientific, Rockford, IL) and equal amount of protein were denatured with 2-mercaptoethanol (Sigma-Aldrich, St. Louis, MO) following standard Western blotting protocol. Proteins were resolved on 4-15% TGX gels (Bio-Rad, Hercules, CA), and transferred to nitrocellulose membranes. Membranes were blocked with 3% BSA for 1 h and probed for Cyclophilin B and GFP at 4° C. overnight. Membranes were washed thrice before incubation with HRP-conjugated secondary antibodies for 1.5 h. Protein bands were visualized by soaking stained membranes in chemiluminescent substrate (Thermo Scientific, Rockford, IL) and imaged using the ChemiDoc system.

### In Vivo Evaluation of the Delivery Strategy

### Orthotopic GL261 Glioblastoma Mouse Models

All animal procedures were performed as approved by the Johns Hopkins Animal Care and Use Committee. CX3CR-1GFP mice were housed at constant temperature and humidity (20±1° C., 50±5% humidity). Animals were anesthetized through intraperitoneal injections with a cocktail of 2.5% xylazine (VetOne, Boise, MO), 25% ketamine (Henry Schein, Melville, NY), and 14.2% ethanol (Sigma Aldrich, St. Louis, MO) in saline for all procedures. GL261 cells were collected at a concentration of 50,000 cells/μL immediately before inoculation and kept on ice during surgery. An incision through the scalp was created and a micro-drill (Braintree Scientific, Braintree, MA) was used to drill a hole through the skull 1 mm posterior to the bregma and 1.5 mm lateral to the centerline. GL261 cells were inoculated through the opening using a 2 μL syringe (Hamilton, Reno, NV) at a rate of 0.2 μL/min for 2 μL of cell suspension per animal. The incisions were closed with suture (Ethicon) and antibiotic ointment was applied.

### Administration of D-scRNA and D-siGFP Conjugates

Two weeks post-inoculation, the incisions were re-opened, and the animals received an intratumoral injection of 2 μg of D-scRNA, D-siGFP or free siGFP on a nucleic acid basis. To determine the uptake and efficacy, animals were anesthetized with isoflurane at 24 h and 48 h time points and euthanized with cardiac perfusion of PBS.

### Immunohistochemistry and Optical Imaging

Extracted brains were immediately fixed in 4% paraformaldehyde, stored in 4° C. overnight, and subjected to a sucrose gradient before cryosection. The organs were processed on a Leica CM 1905 cryostat to obtain 30 mm thick axial sections. Each slide was stained with DAPI (nuclei) and imaged with a confocal LSM 710 microscope (Carl Zeiss; Hertfordshire, UK). Unstained and untreated control brains were used in calibration to avoid background fluorescence and the settings were used without change throughout the study. Both tumors and the corresponding contralateral hemisphere were imaged for analysis with the contralateral hemisphere serving as internal control.

### Statistical Analysis

Data were presented as means±SEM and analyses were performed in Excel 2013 and GraphPad Prism (version 6: La Jolla, CA). Treatment groups across time point or doses were analyzed through two-way analysis of Variance (ANOVA) tests. Significant differences among single groups were determined with Student's t tests: *P<0.05, **P<0.01 and ***P<0.001.

### Results

Synthesis and characterization of Cy5-D-PEG₄-SPDP:Cy5-D-PEG₄-SPDP conjugate was synthesized using PAMAM-G6-OH (D6-OH) dendrimer composed of 256 terminal hydroxyl groups (FIG. 3). After each synthetic step, the product was purified via dialysis in DMF for 24 h to eliminate small molecule impurities followed by water dialysis to remove DMF. 1H NMR (in DMSO-d6) comparison of intermediates and the final conjugates from top to bottom and analytical HPLC traces confirmed the product formation by appearance and disappearance of peaks and showing shifts in the retention times respectively. Molecular weights of all intermediates and final components were determined by MALDI-spectra of PAMAM-G6-OH, Cy5-D, and Cy5-D-PEG₄-SPDP; the degree of conjugation in each step of the synthesis was calculated based on the 1H-NMR and change of molecular weights measured by MALDI-TOF. The synthesis of compound 1 was carried out starting from commercially available D6-OH in methanol (13.75% w/w), which was dried under reduced pressure, followed by the dissolution in water and lyophilization. Trace amount of methanol and water in dendrimer were completely removed as they interfere with coupling steps. The lyophilized mono-functional D6-OH was first functionalized with Boc protected amine by treatment of Boc-GABA-OH under EDC·HCl and 4-DMAP in DMF for 36 h at room temperature to yield the product, Boc protected bifunctional dendrimer. The completion of reaction was monitored by HPLC and the residue was dialyzed by 3.5 kDa membrane against ultrapure water for 24 h to remove low molecular weight impurities via selective diffusion across the semi-permeable dialysis membrane. 1H NMR of dendrimer (3) depicted the appearance of tert-butyl protons of Boc group at 61.3 ppm as a singlet along with GABA methylene protons at δ 1.6 ppm. The peak at δ 3.9 ppm is for the methylene protons of the dendrimer next to hydroxyl groups once converted to ester and amidic protons from GABA linker also appeared at δ 6.8 ppm. Then the Boc groups were de-protected under mild acidic condition using trifluoroacetic acid (TFA) in dichloromethane (DCM) 1:4 to obtain bi-functional dendrimer (4). The excess TFA was removed by co-evaporation with methanol and resulted crude product was used for next step without further purification. The complete disappearance of Boc protons was confirmed by 1H NMR while no ester hydrolysis was observed under this condition. The total number of amine groups was maintained at ˜10. Then bifunctional dendrimer was treated with fluorescent dye Cy5 to yield dendrimer 4 with ˜1-2 successful Cy5 attachment at dendrimer surface. 1H NMR showed the appearance of Cy5 signals in the aromatic region (1H NMR (DMSO-d6, 500 MHz) characterized dendrimer conjugates, D-GABA-Boc, D-GABA-NH₂, Cy5-D, Cy5-D-PEG₄-SPDP (in DMSO-d6 and D₂O), to indicate the appearance or disappearance of characteristic signals.) and HPLC retention time was shifted from 19.0 to 17.5 min confirming the product formation. After Cy5 attachment rest of amine groups were reacted with hetero-bi functional 3-(2-pyridyldithio)propionamido-PEG4-NHS ester (NHS-PEG₄-SPDP) linker. This hetero-bi functional linker used to form a disulfide-reduction-sensitive linker between dendrimer and siRNA through classical thiol-disulfide interchange by siRNA-SH. This bond is relatively stable in the serum and cleavable in the reductive cytoplasmic milieu. Analytical reverse phase-High-performance liquid chromatography (HPLC) was conducted to measure the purity of products. The sizes of components were measured by dynamic light scattering (DLS) using Zetasizer. The mean hydrodynamic diameter of both D-OH and D-siRNA in water were ˜6 nm and show no significant change after modification.

### Synthesis and Characterization of Dendrimer-siRNA Conjugate 1

The synthesis of dendrimer-siGFP (D-siGFP) is shown in the FIG. 4. The delivery efficiency of siRNA bioconjugates depends on the site of conjugation, cleavability of the linker, length of the spacer arm, and the bio-physical properties of the conjugated molecule. siRNA is a duplex consisted of two complementary strands, sense and antisense with terminal phosphate groups that can be used for chemical conjugation. There are four terminal ends that can be used as conjugation sites. Upon cellular uptake, the antisense strand with complementary sequence to a target mRNA is incorporated into RISCs. The 5′ of antisense strand is especially important in the initiation of RNAi mechanism. Therefore, the 5′ and 3′ ends of the sense or passenger strand and the 3′ end of the antisense strand are potential sites for conjugation with modifications over sense strand being more favorable to minimize changes in silencing potency. As a result, disulfide thiol-modifier for introducing sense 5′ thiol (—SH) linkage was used for this study. The SH-modified siGFP can be used to form reversible disulfide bonds, ligand-S—S-siGFP or irreversible bonds with various activated accepting groups. The thiol-modified siGFP in protected form used here prevents the formation of dimers. Prior to use, the thiol-modified (S—S) siGFP was reduced to sulfhydryl (—SH) for further conjugation. The dithiol modified siGFP was treated with 100 mM of dithiothreitol (DTT) to quantitatively reduce diulfide bonds, resulting in sulfhydryl groups for further conjugation with dendrimer. HPLC analysis indicated near quantitative reduction of dithiol group and removal of excess DTT prior to next reaction step. The resulted sulfhydryl group in the sense 5′ end of siGFP was then reacted with dendrimer-PEG₄-SPDP (5) to form desired D-siGFP (1) conjugate via a sulfhydryl exchange reaction. The 2-pyridylthio group reacts with sulfhydyls under the neutral pH by displacement of electron stabilized 2-pyridyl group with thiol compound. This thiol-exchange reaction is commonly used in many crosslinking and conjugation reactions where SPDP readily undergo an interchange reaction with a sulfhydryl group to yield a single disulfide product. The newly formed disulfide bond between dendrimer and siRNA is susceptible to reduction under acidic conditions. The resulted D-siGFP was passed through GE Healthcare SEPHADEX® G-25 column and concentrated by ultrafiltration. The molecular weight was determined by the MALDI-TOF TOF spectrum of Cy5-D-siGFP, showing peak at mass of 72908 Da and HPLC trace. The purity of the product was confirmed by the HPLC of Cy5-D-siGFP at 210, 260, and 650 nm. Further, the successful synthesis of D-siGFP was confirmed by gel retardation. The clear single band from D-siGFP was retained at a distance to corresponding 150 bp marker on the 10% TBE-Urea gel, with an estimated size of 90 kDa in size, as determined by gel retardation of naked siGFP, and D-siGFP.

### Serum Stability of Chemically Conjugates D-siRNA

Protection of the nucleic acid payload against nucleases is crucial for the success of RNAi therapy. Therefore, the ability of D-siGFP to deliver the intact payload was validated against RNase III, an endonuclease, under reducing (1 mM DTT) and non-reducing (0 mM DTT) conditions. Under non-reducing conditions, naked siGFP was degraded by RNase III in less than 2 h whereas D-siGFP remained stable up to 48 h; D-siGFP and siGFP were incubated with RNase III nuclease under non-reducing and reducing conditions. Under non-reducing conditions, Naked siGFP is degraded by 30 min while D-siGFP retains up to 48 h. Under reducing conditions the nucleic acid payload of both siGFP and D-siGFP was rapidly released from the dendrimer platform and both the naked siGFP and D-siGFP were rapidly degraded in 15 min. In addition, siGFP and D-siGFP were incubated in human plasma to mimic serum stability in vivo. The band for D-siGFP remained at 150 bp, indicating the lack of plasma protein binding while the siGFP band shifted drastically from 20 bp to 150 bp in as fast as 1 h with significant protein binding. Significant plasma protein binding to free siGFP occurred as early as 1 h in 37° C. in human plasma and the amount bound to protein increased 48 h after incubation. No significant protein adsorption was observed in D-siGFP.

### In Vitro GFP Knockdown in HEK-293T Cells

The delivery of siRNA into cells using chemically conjugated dendrimer-based platform was assessed in vitro using a GFP expressing HEK293T cell line. The HEK293T cells expressed a destabilized form of GFP (GFPd2) with a half-life of ˜2 h which is comparable to many proteins in an in vivo environment. For time dependent uptake studies, HEK 293T cells were treated with Cy5-D-siGFP-Cy3 and cells were incubated for 48 h. The cells were washed prior to imaging at each time point and the treatment media reapplied after each imaging session. As early as 6 h, D-siGFP showed intracellular accumulation; cellular uptake and dose dependent gene knockdown of Cy5-D-siGFP was observed by confocal microscopy images of Cy5-D-siGFP-Cy3 cell uptake into HEK293T cells. At 24 h post treatment, diffuse Cy3-siRNA signal is detected while Cy5-dendrimer signal is punctate; the Cy5-Dendrimer signal and Cy3-siRNA signal is co-localized.

Naked siGFP did not accumulate in HEK293T cells to any appreciable degree. Confocal microscopic images revealed that the dendrimer Cy5 distributed in the cytoplasm of the HEK293T while dendrimer Cy5 signal is colocalized with siRNA Cy3 signal at 24 h post-treatment. The colocalization of Cy5 and Cy3 signal was confirmed by a merged (pink) signal upon merging the channels.

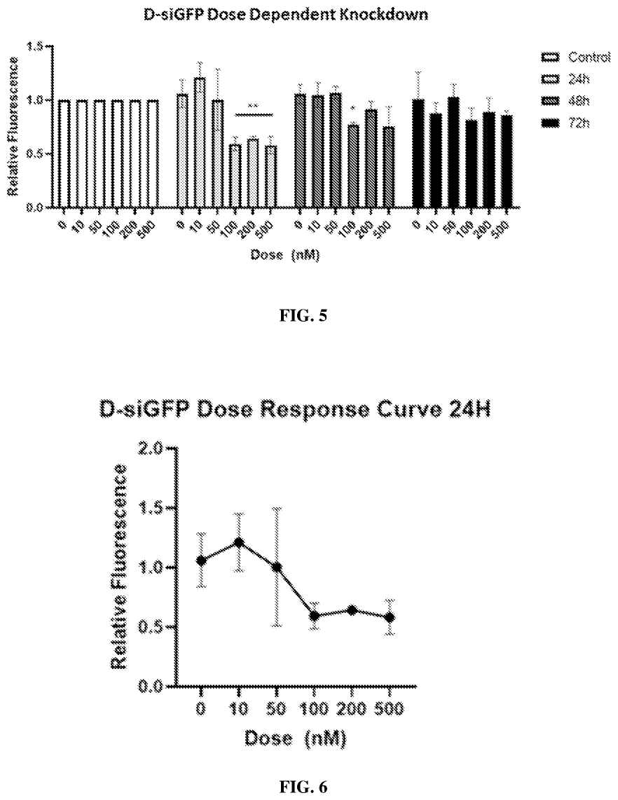

To assess GFP knockdown, HEK293T cells were seeded 24 h before treatment and culture media was replaced with OPTIMEM® immediately prior to treatment. Cells were treated with D-siGFP at five different concentrations including 10, 50, 100, 200, 500 nM. GFPd2 expression was estimated by relative fluorescence intensity using background adjusted intensity in the GFP channel and normalized to internal controls at 0 h time point. The optimum knockdown was reported at 24 h post-transfection. According to live-cell images, a significant, time-dependent knockdown of GFP protein was reported at concentrations greater than 50 nM with a peak of ˜40% knockdown at 24 h. GFP concentration returned to normal over a period of 72 h. After 48 h, cells were collected and lysed for Western blotting. IC50 value was estimated using the dose response curve of D-siGFP at 24 h post-transfection (FIGS. 5 and 6).

Delivery of siRNA Using Commercial Transfection Reagents

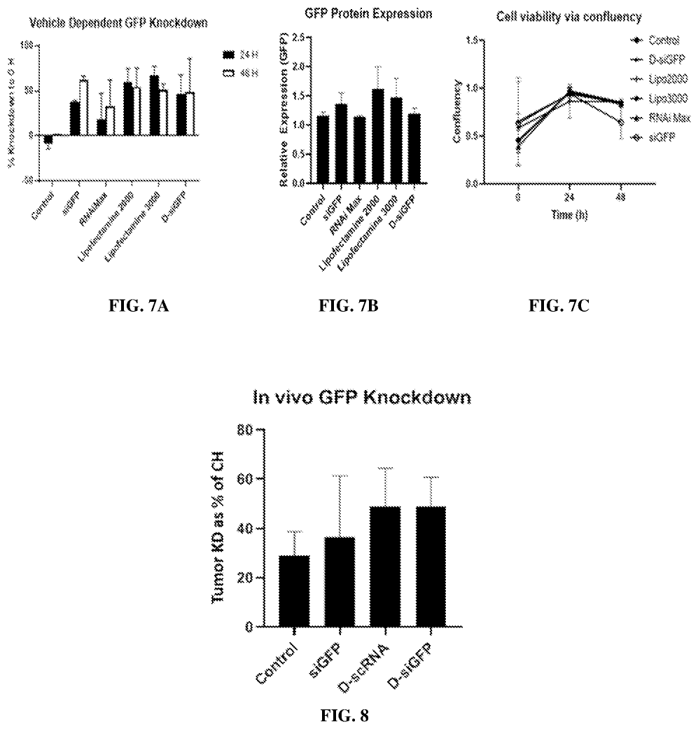

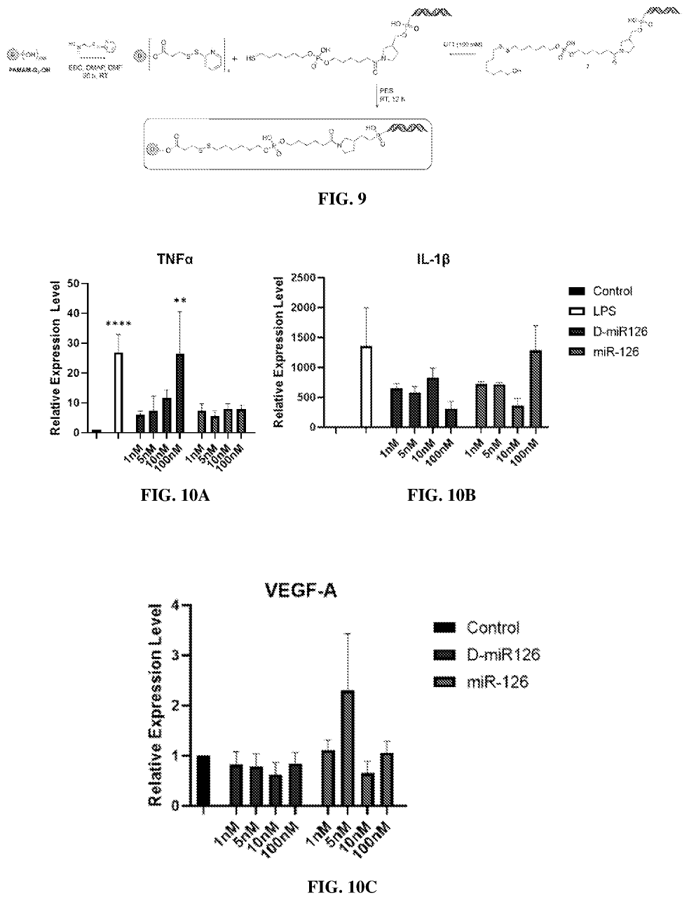

The effects of commercial transfection reagents, LIPOFECTAMINE® 2000, LIPOFECTAMINE® 3000 and RNAi Max were assessed as comparative controls. In image analyses of GFP fluorescence, all commercial platforms resulted in some knockdown of GFPd2 expression (FIGS. 7A-7C) with conditions and nucleic acid loading optimized to each vehicle. Live-cell images were also analyzed for confluency as an indirect measure of cytotoxicity; there is no significant toxicity observed across all systems. Direct measures of relative GFP expression via Western blots did not result in any statistically significant differences between delivery systems but a trend can be observed where the LIPOFECTAMINE® systems and naked siGFP resulted in a more inconsistent effect on GFP production. The discrepancy between GFP fluorescence detected from image analysis and actual GFP protein production suggested LIPOFECTAMINE® systems may release their payloads in a burst release, achieving the observed knockdown in image analysis and the subsequent rise in GFP protein when siGFP underwent degradation. On the other hand, RNAi Max and D-siGFP may exhibit a slower release profile, resulting in the prolonged knockdown of GFP production.

### In Vivo Study of Cy5-D-siGFP in GL261 Glioma

To validate the D-siGFP conjugate as an effective siRNA transfection agent, D-siGFP was injected intratumorally in an orthotopic glioblastoma mouse model. Intratumoral injection was chosen due to its prevalence in gene therapy applications and to demonstrate efficacy and uptake of D-siGFP without waste. CX3CR-1GFP mice were first inoculated with 2×10⁵ GL261 cells two weeks prior to intratumoral injections of dendrimer conjugates, allowing the tumors to grow to sufficient size.

For uptake studies, the dual labeled conjugate, Cy5-D-siGFP-Cy3 was administrated intratumorally and organs were extracted 24 h post-injection. Confocal images of the tumor, the tumor border, and the contralateral side were obtained and analyzed through Zen 2011 software. The dual labeled D-siRNA was distributed diffusely within the tumor, was only observed within the tumor parenchyma, and was absent in the contralateral hemisphere. Cy5-D-siRNA-Cy3 selectively targeted TAMs and knockdown genes in GFP transgenic GL261 mouse model; D-siGFP is retained in the tumor following intratumoral administration and uptake of D-siGFP is concentrated around tumor-associated macrophages (TAMs). Some Cy5 (dendrimer) and Cy3 (siGFP) signals co-localized with one another, suggesting the delivery of intact D-siGFP conjugate. In addition, Cy3 signal was also present and colocalized with GFP signals expressed by TAMs, indicating uptake of siGFP. Interestingly, only some of the Cy3 signal co-localize with both GFP and Cy5, indicating that only a small portion of D-siGFP conjugate remain intact after cellular uptake. The majority of Cy3 and Cy5 signal dissociate with each other, indicating that the siGFP sequence is released from the dendrimer vehicle after cellular uptake.

Next, the knockdown of GFP expression was investigated. Tumor-bearing animals were injected with D-siGFP, D-scRNA or free siGFP, and the organs collected at 24 h and 48 h post-injection. The contralateral hemisphere was used as internal controls and D-scRNA was used as vehicle controls. There is a 50% knockdown in GFP fluorescence D-siRNA administrated tumors compared to contralateral hemisphere as internal control (FIG. 8). In untreated animals, GFP fluorescence decreased by 20% with tumor inoculation.

### SUMMARY

siRNA plays a key functional role in gene-silencing process by pairing with specific mRNA sequences and degrading them through RISC complexes, resulting in the knockdown of specific protein expression. Therefore, the delivery of intact siRNA sequences to the target cell is crucial for the success of RNAi therapies. A facile dendrimer-siRNA conjugation strategy was developed based on biocompatible hydroxyl terminated PAMAM dendrimer which produces environment responsive nanoparticle conjugates with precise nucleic acid loading and inherent targeting to areas of inflammation.

Synthesis was conducted under mild reaction conditions via tunable synthetic route using affordable synthetic materials and simple purification techniques. The stimulus responsive linker chemistry used herein plays a key role in releasing the payload to the intracellular environment specifically while dendrimer conjugation improves nuclease resistance and efficiency of delivery. The reported half-life of naked siRNA in serum ranges from several minutes to 1 h, while the results suggest that the chemically conjugated D-siRNA improves the stability by delaying serum degradation from 30 min to 48 h without compromising the knockdown efficiency.

This is a remarkable advancement in in vivo potency of RNA interference. Furthermore, D-siRNA underwent immediate disulfide bond reduction with an in vitro reductant, suggesting that the cytosolic environment is capable of triggering the release of siRNA. The study herein also highlighted that the chemical conjugation of siRNA to dendrimer does not impair gene-knockdown activity. In in vitro settings, it was revealed that covalently conjugated D-siRNA may produce a sustained knockdown by slowing release of the nucleic acid payload.

The in vivo proof-of-principle results revealed that covalently conjugated D-siGFP is capable of producing targeted gene knockdown effect. The relatively modest knockdown is reported for both in vitro HEK293T cells and in vivo brain tumor model (˜50%). According to confocal analysis, D-siGFP localized within tumor associated macrophages and released the payload intracellularly while there is virtually no uptake in other cell populations or in the contralateral hemisphere. Chemically conjugated siRNA does not affect the intrinsic properties of PAMAM dendrimer in achieving high tumor specificity in orthopedic GL261 mouse model and is capable of producing high gene knockdown compared to free siGFP.

Covalent conjugation of siRNA to dendrimer greatly enhanced serum half-life and bioavailability, protecting the payload from protein adsorption and enzymatic degradation. The D-siRNA conjugate effectively delivered siRNA to cells both in vitro and in vivo while efficiently knocking down the targeted gene. In in vitro studies, D-siGFP achieved similar magnitude of knockdown as RNAi Max and LIPOFECTAMINE® systems. In in vivo studies, D-siGFP preferentially localized within the tumor parenchyma, released its payload intracellularly, and achieved gene silencing effect in GFP expressing tumor associated macrophages. These results demonstrate that a facile dendrimer based covalent conjugation strategy provides an efficient and safe approach for the clinical translation of siRNA therapeutics.

### Example 3: Dendrimer-miR126 Conjugates for the Targeted Suppression of Choroidal Neovascularization

### Material and Methods

### Chemicals and Reagents

Hydroxyl-terminated, ethylenediamine-core PAMAM dendrimer (generation 6, pharmaceutical grade) in methanol solution was purchased from Dendritech (Midland, MI, USA). Prior to use, the dendrimer solution was evaporated on a rotary evaporator. Dialysis membrane (MWCO 1 kDa) was purchased from Spectrum Chemicals (New Brunswick, NJ, USA). Thiol modified miR-126:
sense: 5′-UCGUACCGUGAGUAAUAAUGCG-3′ (SEQ ID NO: 4);
antisense: 5′-CGCAUUAUUACUCACGGUACGA-[Thiol C6 S—S]-3′ (SEQ ID NO: 5), and the Cy3 labeled equivalent were purchased from Bio-Synthesis (Lewisville, TX, USA). Bio-Spin P-30 Gel Columns and 15% TBE-Urea Pre-cast gels were purchased from Bio-Rad (Hercules, CA, USA). Amicon Ultra Centrifugal Filters (MWCO 10 kDa), GelRed nucleic acid dye, and anhydrous N,N′-dimethylformamide (DMF) were purchased from Sigma-Aldrich (St. Louis, MO, USA). Deuterated solvents (DMSO-d₆), methanol (CD₃OD), and water (D₂O) were also purchased from Sigma-Aldrich. dsRNA ladder was purchased from New England BioLabs (Ipswich, MA, USA). Dulbecco's modified Eagle medium (DMEM, low glucose with L-glutamine were purchased from ThermoFisher (Waltham, MA, USA). Human Microvascular Endothelial Cells and the required media kit were purchased from Lonza (Basel, Switzerland). Phenol Red-free Matrigel was purchased from Corning (Tewksberry, MA, USA).

### Instrumentation

Structures of intermediates were analyzed using proton nuclear magnetic resonance (¹H NMR) spectroscopy using a Bruker 500 MHz spectrometer (Bruker Corporation, Billerica, MA, USA) at ambient temperatures. Chemicals shifts were relative to an internal standard of tetramethylsilane and reported in ppm. The chemical shifts of residual protic solvents D₂O (¹H, δ 4.79 ppm), and DMSO-d₆ (¹H, δ 2.50 ppm) were used for chemical shift calibration.

The purity of intermediates and dendrimer-miR126 conjugate were analyzed using high performance liquid chromatography (HPLC). The HPLC instrument (Waters Corporation, Milford, MA, USA) was equipped with a 1525 binary pump and an in-line degasser AF. The instrument was equipped with a 717 plus autosampler and had two detectors: a 2998 photodiode array detector and a 2475 multi k fluorescence detector. The instrument was interfaced with Waters Empower software. HPLC samples were run on a C18 symmetry 300, 5 μm, 4.6×250 mm column from Waters. Chromatograms were recorded at 210 nm (dendrimer absorption), and 260 nm (nucleic acid absorption). A gradient flow HPLC method was used starting with 90:10 (Solvent A: 0.1% TFA and 5% ACN in water; Solvent B: 0.1% TFA in ACN), gradually increasing to 50:50 (A:B) at 30 min, and finally returning to 90:10 (A:B) at 40 min with a constant flow rate of 1 mL/min.

### Synthesis of Dendrimer Conjugates

### Synthesis of Dendrimer-PDP, 1

Succinimidyl 3-(2-pyridyldithio)propionate (SPDP, 14 mg, 0.068 mmol) was added to a stirring solution of D-OH (200 mg, 0.0034 mmol) in anhydrous DMF. The reaction was allowed to continue at room temperature for 24 h. The mixture was diluted with DMF and dialyzed against DMF using a 1 kDa cutoff dialysis membrane. DMF was changed every 4 h for 12 h followed by 12 h dialysis against water with frequent water changes. The resultant aqueous solution was lyophilized to afford D-PDP as an off-white powder (80% yield).

¹H NMR (500 MHz, DMSO) δ 8.30 (d, aromatic 4H), 8.06-7.79 (m, internal amide 510H), 7.01 (m, aromatic 5H), 4.73 (s, surface OH, 235H), 4.06 (s, ester linked, 12H), 3.40 (d, dendrimer —CH₂), 3.33 (d, dendrimer —CH₂), 3.18-3.11 (m, dendrimer —CH₂), 2.64 (s, dendrimer —CH₂), 2.43 (s, dendrimer —CH₂), 2.20 (s, dendrimer —CH₂). Retention time: 18.00 min.

### Synthesis of Boc-GABA-Dendrimer-PDP, 2

Compound 1 (100 mg, 0.0016 mmol) was dissolved in 3 mL of DMF followed by the addition of Boc-GABA-OH (1 mg, 0.005 mmol) and DMAP (1 mg, 0.008 mmol). The solution was stirred for 10 minutes at room temperature prior to the addition of EDC·HCL (2 mg, 0.013 mmol). The reaction mixture was left at room temperature under constant stirring for 24 h before transferring the crude product to 3 kD cut-off cellulose dialysis tubing and dialyzed against DMF for 12 h. The product was then dialyzed against water for an additional 24 h. The resulting aqueous solution was frozen and lyophilized to yield product 2 as a hygroscopic white solid (95 mg, 94%). 1H NMR (500 MHz, DMSO-d6) 8.12-7.78 (m, internal amide H), 7.01 (m, aromatic 5H), 6.59 (s, GABA amide H, 10H), 4.73 (s, surface OH, 233H), 4.06 (s, ester linked, 16H), 3.40 (d, dendrimer —CH2), 3.33 (d, dendrimer —CH2), 3.18-3.11 (m, dendrimer —CH2), 2.64 (s, dendrimer —CH2), 1.64-1.59 (m, GABA linker —CH¬2¬, 6H), 1.36 (s, Boc group, 20H).

### Synthesis of NH₂-GABA-D-PDP, 3

Deprotection was carried out by adding compound 2 (95 mg, 0.0015 mmol) to a mixture of TFA/DCM (3:4) and stirring vigorously for 12 h. The suspension was diluted with methanol and concentrated in vacuo, repeating the process thrice to remove excess TFA. The crude product was used without further purification.

### Synthesis of Cy5-D-PDP, 4

Compound 3 (108 mg, 0.0018 mmol) was dissolved in DMF and treated with DIPEA, adjusting the pH of the mixture (pH˜7.0-7.5). Cy5-NHS ester (2.8 mg, 0.0027 mmol, 1.5 eq.) was added and stirred for 12 h at room temperature. The crude mixture was dialyzed against DMF for 12 h and against water for 24 h. The aqueous solution was frozen and lyophilized to yield product 4 as a blue powder (yield 86%). ¹H NMR (500 MHz, DMSO-d₆) 8.12-7.78 (m, internal amide H), 7.30 (s, Cy5 H), 7.01 (m, aromatic 5H), 4.73 (s, surface OH, 168H), 4.06 (s, ester linked, 16H), 3.40 (d, dendrimer —CH₂), 3.33 (d, dendrimer —CH₂), 3.18-3.11 (m, dendrimer —CH₂), 2.64 (s, dendrimer —CH₂), 1.64-1.59 (m, GABA linker —CH2). Retention time: 18.07 min.

### Synthesis of Dendrimer-miR126, 5

Thiol-modified miR-126 and its Cy3-labeled equivalent were deprotected according to manufacturer protocol. In brief, the lyophilized miR-126 was re-suspended in an aqueous solution of triethylamine (TEA, 2%) and dithiothreitol (DTT, 50 mM). The solution was kept at room temperature for 10 minutes and extracted 4 times with ethyl acetate to remove DTT.

To a stirring solution of 1 (1 mg, 0.00017 mmol) in diethyl pyrocarbonate treated (DEPC-treated) water (Invitrogen, Rockland, IL, USA) was added the deprotected miR-126 (0.5 mg, 0.00034 mmol). The solution was stirred for 48 h and transferred to 3 kDa cut-off AMICON® centrifugal filters. The solution was washed and concentrated thrice through centrifugation with DEPC-treated water. The concentrated solution was passed through P-30 gel column to remove unreacted miR-126. Retention time: 17.33 min

### Synthesis of Cy5-D-miR126-Cy3, 6

Cy3 labeled, thiol-modified miR-126 was deprotected according to the above steps then added to an aqueous solution of 4. The solution was stirred for 48 h and transferred to 3 kDa cut-off Amicon centrifugal filters. The solution was washed and concentrated thrice, then passed through P-30 gel column to remove unreacted nucleic acid.

### Gel Electrophoresis

Purified D-miR126, thiol-modified miR-126, and dsRNA ladder were mixed with GELRED® nucleic acid stain and glycerol (10% v/v), and loaded on 15% TBE-Urea gel. The gel was subjected to constant voltage (120V) and visualized with CHEMIDOC® Imaging System (Bio-Rad, Hercules, CA).

### MALDI-TOF Analysis

Matrix 2-4′6′-Trihydroxyacetophenone monohydrate (THAP) was dissolved in Acetonitrile:Water mixture (1:1) with 0.1% trifluoroacetic acid at a concentration of 10 mg/mL. 5 μL of D-miR126 was deposited on the MALDI sample plate at a concentration of 1 μg/μL followed by 2 μL of the matrix mixture. 2 μL of D-PDP was deposited on the sample plate at concentration of 1 μg/μL followed by 2 μL of the matrix mixture. The samples were allowed to air dry overnight and analyzed through reflective-positive mode of MALDI-TOF MS.

### Cell Line

Human microvascular endothelial cells (HMECs) were acquired from Lonza and cultured in EGM™-2 endothelial cell growth medium (Lonza). BV-2 murine macrophage were provided by the Children's Hospital of Michigan Cell Culture Facility and cultured in Dulbecco's modified Eagle's medium (DMEM; Gibco Laboratories) supplemented with 10% FBS and 1% penicillin/streptomycin. All cell lines were maintained at 37° C. and 5% C02 under humidified atmosphere.

### In Vitro Evaluation of D-miR126 Efficacy

### HMEC Vessel Formation Assay

HMECs were seeded onto 12-well plates at a density of 1×10⁵ cells/mL and allowed to grow to confluency. The cells were then co-incubated with D-miR126 and miR-126 and full-serum media for 24 h. After 24 h of treatment, cells were collected by detaching them with trypsin, collecting the resulting cell suspension, and centrifuging at 300 g for 5 min. Tube formation assay was performed according to protocol provided by Lonza.

In brief, on the day of the assay, 96-well plates were coated with 75 μL of phenol-free MATRIGEL® and left to polymerize at 37° C. for 20 min. The cell pellets were re-suspended with 300 μL of media and 75 μL of the solution (400,000 cells/mL) were seeded onto each MATRIGEL®-coated wells. After 7 h, the resultant cellular network was imaged on a Zeiss Axiovert 200 phase-contrast microscope (Carl Zeiss, Oberkochen, Germany) and analyzed through Angiogenesis Analyzer-ImageJ plug-in (Carpentier, G. et al., Sci. Rep. 10, 11568 (2020)).

PCR Analysis of Pro-Inflammatory and Pro-Angiogenic mRNA Expression

HMECs were incubated with D-miR126 or miR-126 in full-serum conditions for 24 h prior to sample collection. To induce a pro-inflammatory phenotype, BV2 murine macrophage were first stimulated with LPS (100 ng/mL, Sigma-Aldrich) for 3 hours in serum-free media then co-treated with LPS (100 ng/mL) and D-miR126 or miR-126 for 24 h.

HMEC and BV2 samples were then collected with TRIzol for polymerase chain reaction (PCR) analysis. In brief, samples were subjected to a freeze-thaw cycle with TRIzol followed by the addition of 200 μL of chloroform (Thermo Fisher Scientific). The samples were shaken and placed in ice for 15 min. To aid in the separation of aqueous and organic layers, samples were centrifuged for 15 min at 15,000 g. The aqueous solution was collected and isopropanol was added to each sample (500 μL; Thermo Fisher Scientific). Samples were centrifuged again for 15 min at 15,000 g then washed with 75% ethanol in DEPC-treated water.

RNA content was determined through Nanodrop and equivalent amount of RNA from each sample were converted to complementary DNA (Applied Biosystems, Foster City, CA). PCR analysis was performed on STEP ONE PLUS® real-time PCR system (Applied Biosystems) with Fast SYBR Green reagent. PCR primers for VEGF-A, GAPDH, and IL-1β were obtained from Bio-Rad Laboratories (Hercules, CA). Primers were purchased from Integrated DNA Technologies (Coralville, IA). Primers for TNFα were:

forward:
(SEQ ID NO: 6)
CCA GTG TGG GAA GCT GTC TT;
and
reverse:
(SEQ ID NO: 7)
AAG CAA AAG AGG AGG CAA CA.

### In Vivo Evaluation of D-miR126 in Laser-Induced Choroidal Neovascularization (CNV) Mouse Model

### Laser-Induced CNV Mouse Model

All animal procedures were approved by the Johns Hopkins Animal Care and Use Committee. C57BL/6J mice were obtained through The Jackson Laboratory (Bar Harbor, ME, USA) at the age of 5-7 weeks, and housed at constant temperature and humidity (20±1° C., 50±5% humidity). Prior to laser induction of CNV, a cocktail of ketamine/xylazine/acepromazine (100 mg/kg of ketamine, 20 mg/kg of xylazine, and 3 mg/kg of acepromazine) was injected intraperitoneally to anesthetize the animals. Animals were then treated with a drop of topical 2.5% Phyenylephrine Hydrochloride ophthalmic solution followed by 0.5% Tetracaine hydrochloride ophthalmic solution for pupil dilation. A Micron III SLO (Phoenix Research Labs, Pleasanton, CA) was used to image the fundus and focus the attached laser system (Phoenix Research Labs). Four equidistantly placed laser burns in the Bruch's membrane were created with a laser power setting of 240 mW at a duration of 70 ms.

Administration of D-miR126 and miR-126

Immediately after CNV induction, D-miR126, miR-126, or saline sham were administered to the animals. In brief, a 30G insulin syringe was used to create an injection opening in the sclera. A 10 μL Hamilton syringe was then used to administer the treatment directly into the vitreous cavity. After treatment, animals were given topical ocular antibiotic (gentamicin and prednisolone acetate ophthalmic ointment) to prevent infection. Animals were then sacrificed at set time points post treatment (7 and 14 days) for imaging and biochemical analysis.

For biodistribution studies, 1 μg of Cy5-D-miR126-Cy3 or miR-126-Cy3 on a nucleic acid basis (at concentration of 1 μg/μL) were administered to the animals in accordance to the above steps. Tissues were extracted at set time points (1, 3, 5, 7, 14 days post-treatment).

### Immunohistochemistry and Imaging

At set time points, animals were sacrificed and the enucleated eyes fixed in 4% paraformaldehyde for 1 h. The choroid and retina are then dissected out. Tissues were blocked and permeabilized through 2 h incubation with a solution of 5% normal goat serum, 0.3% Triton X-100, and 1% bovine serum albumin at room temperature under constant agitation. To visualize macrophage, tissues were stained with anti-Iba1 antibody (1:100; FUJIFILM® Wako Chemicals, Osaka, Japan) followed by ALEXA FLUOR® 405-labeled goat anti-rabbit secondary antibody (1:200; Abcam, MA, USA). Blood vessels were stained with FITC-labeled isolectin (GS IB₄) (1:100; Life Technologies, Eugene, OR, USA).

Flat-mounts were created by making four radial incisions on the tissue and mounting the relaxed tissues on coverslips. CNV formation was imaged with confocal 710 microscope (Carl Zeiss, Oberkochen, Germany) for biodistribution, and with an Axiovert phase contrast microscope for area calculation.

### PCR and ELISA Assay

Eyes were dissected immediately after enucleation without fixation. The choroid was collected and stored at −80° C. prior to analysis. For ELISA analysis, choroids were immersed in T-PER protein extraction buffer (Thermo Fisher Scientific) and homogenized with 0.9-2.0 mm stainless steel beads on a Bullet Blender Storm tissue homogenizer (Next Advantage Inc., Averill Park, NY). The supernatant was centrifuged to collect the aqueous solution. The samples were stored in −80° C. and used without further processing for ELISA detection of VEGF-A levels.

For PCR, choroids were immersed in TRIzol, homogenized with steel beads, and filtered through Corning COSTAR SPIN-X® centrifuge tube filters (Sigma-Aldrich) to remove tissue solids. The RNA was isolated following the previously described protocol and the RNA concentration was determined through Nanodrop. Equivalent amounts of RNA were converted to complementary DNA (cDNA) and analyzed with Fast SYBR Green reagent through the STEP ONE PCR® system.

### Statistical Analysis

Data were presented as means±SEM and analyses were performed in GraphPad Prism (version 9; La Jolla, CA). Treatment groups across time point or doses were analyzed through analysis of variance (ANOVA) tests. Significant differences among single groups were determined with Student's t tests: *P<0.05, **P<0.01 and ***P<0.001.

### Results

### Synthesis and Characterization of D-miR126 Intermediates and Conjugates

A reproducible, environment-sensitive conjugation strategy is essential to effectively deliver miRNA to the intracellular environment without reducing its efficacy. This conjugation strategy utilized a proven glutathione sensitive linker to attach miRNA to the dendrimer nanoparticle. The dendrimer surface was first modified with succinimidyl 3-(2-pyridyldithio)propionate, reactive linkers that readily form reducible disulfide bonds with sulfhydryl groups. Successful modification was confirmed by the ¹HNMR spectrum which showed the presence of five aromatic protons at 7.01 ppm and twelve ester-linked protons at 4.06 ppm. Deprotected, thiolated miR-126 was reacted with the modified dendrimer and the reaction was monitored through gel electrophoresis. Formation of dendrimer-miR126 conjugate was confirmed with an increased retention time in TBE-Urea gel at 150 bp when compared to free miR-126 at 27 bp. Gel electrophoresis of D-miR126 exhibited a prolonged retention time corresponding to 150-300 RNA bp (˜90-180 kDa). The presence of a single band in D-miR126 suggested the absence of free nucleic acids. In contrast, the band associated with miR126 traveled further, indicating a smaller bp size. HPLC chromatograph of D-miR126 consisted of a single peak with a retention time of 14.972 min indicating a pure product.

Purified D-miR126 was also subjected to HPLC analysis and the resultant chromatograph consisted of one single peak with a retention time of 14.972 min, suggesting the analyte was pure. In addition, the UV profile of the analyte exhibited two absorption peaks at 200 nm and 260 nm which corresponded to the absorption wavelength of dendrimer and nucleic acid, respectively, suggesting successful incorporation of the nucleic acid to the dendrimer platform.

MALDI-TOF analysis was used to further confirm conjugate formation and determine nucleic acid loading. The mass of D-PDP precursor and D-miR126 conjugate was determined to be 60 kDa and 66 kDa, respectively. All other intermediates and products were characterized using ¹H NMR, HPLC, or gel electrophoresis. To determine whether fluorescence resonance energy transfer (FRET) was possible for the dually labeled conjugate, the sample was excited at 540 nm and the resulting fluorescence intensity measured over the range of 550-720 nm with an RF5301PC spectrofluorophotometer running PANORAMA® 3 software (Shimadzu Scientific Instruments, Columbia, MD). Cy3 emission was determined as intensity from 565-575 nm and Cy5 emission was determined from 665-675 nm. Excitation of the Cy5 fluorophore was not observed in the resulting spectra, suggesting that fluorescence due to FRET was unlikely in subsequent imaging experiments.

In vitro D-miR126 Activity in HMEC and BV2 Cells

BV2 murine macrophage and human microvascular endothelial cell lines were selected to test the efficacy of D-miR126 at reducing pro-inflammatory and pro-angiogenic markers, respectively. LPS stimulated macrophage produced high levels of TNFα and IL-1β compared to untreated controls and the production of these pro-inflammatory cytokines were reduced when co-treated with D-miR126 and miR-126. While the two treatments resulted in similar knockdown of TNFα (˜50%) (FIG. 10A), there appeared to be a dose-dependent knockdown of IL-1β for cells treated with D-miR126 and an inverse dose response for cells treated with miR-126 (FIG. 10B). TNFα response seems to be dose independent.

Anti-angiogenic effect in HMECs were tested in two complementary methods. First, mRNA levels of VEGF-A, an important angiogenic cytokine, was assessed by in miR-126 and D-miR126 treated cells and untreated controls. Compared to controls, HMECs treated with either D-miR126 or free miR-126 resulted in a lower production of VEGF-A (˜20% knockdown), suggesting an inhibition of angiogenic activity (FIG. 10C). VEGF-A repression in HMECs decreased with extremely high or low doses of D-miR126 with the optimal dosage at 5-10 nM. High doses of miR-126 (10-100 nM) repressed VEGF-A expression at the same level as lower doses of D-miR126.

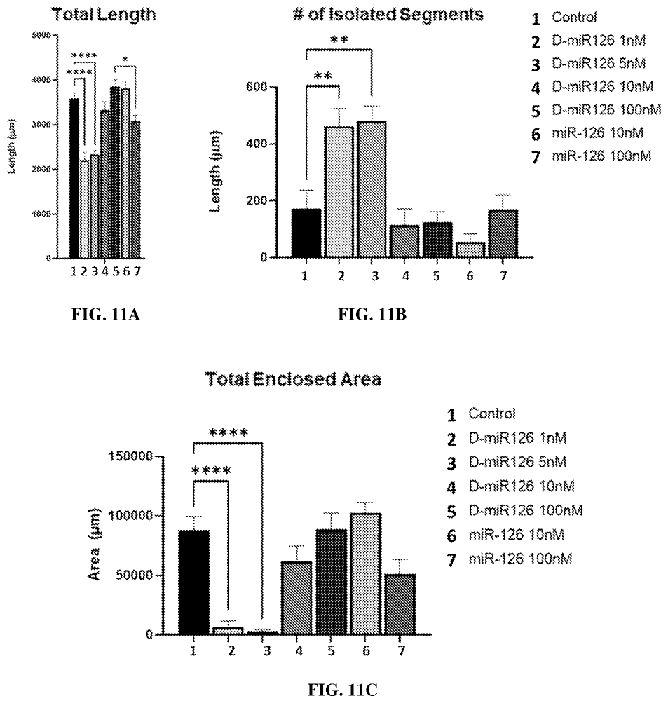

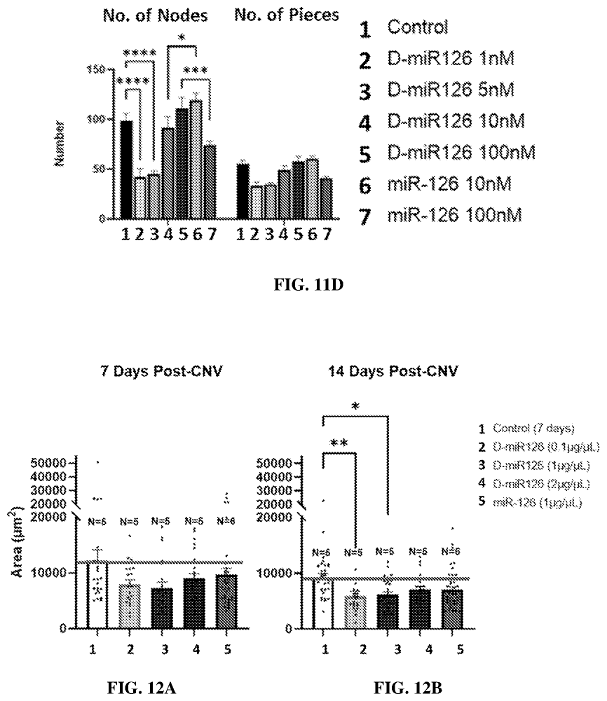

Secondly, angiogenic activity of HMECs were assessed through tube formation assay. Treated and untreated cells were subjected to angiogenic conditions on MATRIGEL® matrix and left to naturally form cell networks. The networks were then analyzed by Angiogenesis Analyzer, a plug-in for Image J. In all measures, cells treated by D-miR126 or miR-126 exhibited a disruption of network formation such as increased isolated fragments, decreased area enclosed by vessels, and decreased network length (FIGS. 11A-11D). Pretreatment with lower doses of D-miR126 repressed the ability of HMECs to form networks on the Matrigel matrix. In contrast, higher doses of free miR-126 was needed to suppress network formation.

### In Vivo Anti-Angiogenesis Activity of D-miR126

Laser-induced CNV mouse models were used to assess the effectiveness of D-miR126 conjugates at reducing CNV formation in vivo. The model produced consistent CNV formation with an area of around 12,000 μm² at day 7 and ˜9,000 μm² at day 14. Mice treated with one dose of D-miR126 or miR-126 on the day of CNV induction produced smaller CNV area. At day 7, D-miR126 treated animals had CNV areas ˜7,000 μm² while miR-126 treated animals had areas ˜8,000 μm². At day 14, D-miR126 treatment reduced CNV area by ˜30% (˜6,000 μm²) compared to the control while miR-126 treatment only reduced CNV area by ˜10% (˜8,000 μm²) (FIGS. 12A-12B). Thus, a single dose of D-miR126 treatment suppresses CNV formation up to 14 days post-administration.

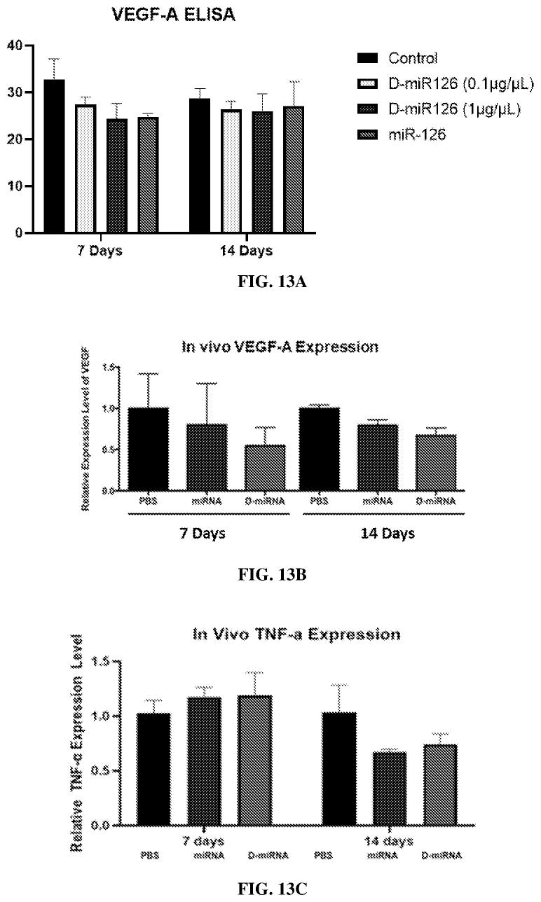

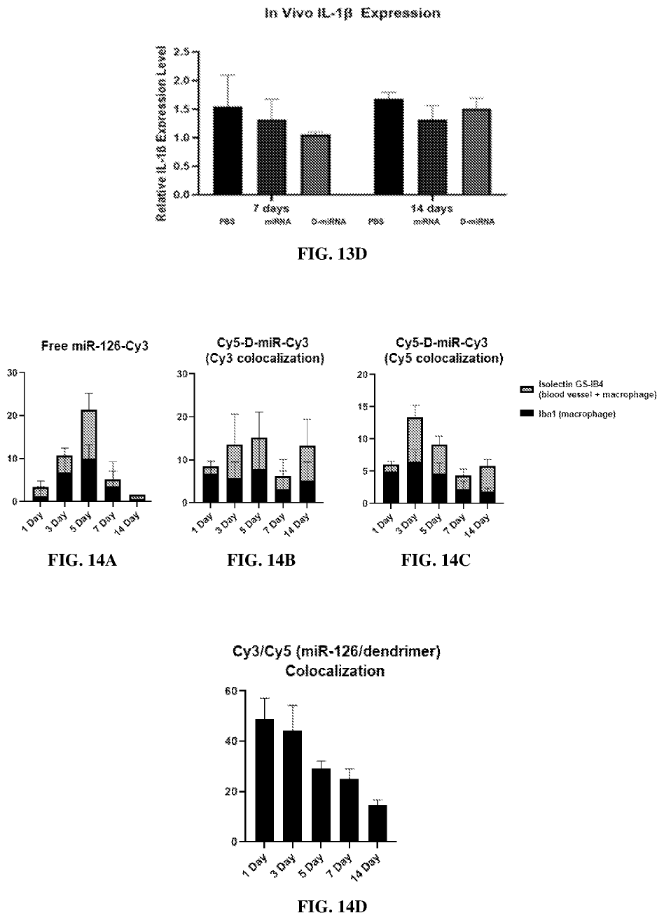

To determine the anti-angiogenic mechanism of D-miR126, VEGF-A levels were measured by PCR and ELISA assays and pro-inflammatory cytokines (TNFα and IL-1β) were measured with PCR. Mice treated with miR-126 and D-miR126 resulted in a significant decrease in VEGF-A protein as measured by ELISA on day 7. Further, D-miR126 significantly reduced VEGF-A protein levels compared to miR-126 treatment. However, by day 14, there were no differences in VEGF levels in treated and untreated animals (FIG. 13A). The trend of VEGF reduction on day 7 was corroborated by mRNA levels measured through PCR but the trend was insignificant due to large variances. Interestingly, though VEGF protein levels appeared similar on day 14, VEGF-A mRNA levels were still reduced in D-miR126 and miR-126 treated animals (FIG. 13B). D-miR126 appeared to be more effective at reducing VEGF-A mRNA.

Animals treated with D-miR126 and miR-126 resulted in attenuation of inflammatory mRNA. On day 7, animals treated with D-miR126 resulted in lower level of IL-1β, but the decrease was not sustained on day 14 (FIG. 13D). Conversely, D-miR126 and miR-126 treatment appeared to only exert its effect on TNFα production at later time points as measured on day 14 (FIG. 13C). TNFα mRNA was elevated at an early time point (7 days) with either D-miR126 or miR-126 treatment but was not statistically significant. Both treatments subsequently suppressed TNFα at 14 days.

### In Vivo Distribution

To assess uptake and distribution, Cy3 labeled miR-126 and dually labeled Cy5-D-miR126-Cy3 were injected into laser CNV mouse models and choroids were collected at 1, 3, 5, 7, 14 days post-injection. Iba1 staining was used to visualize the intracellular environment of macrophage, and isolectin GS-IB4 was used to stain for both blood vessels and macrophages. Intravitreally injected D-miR126 localized to CNV area within 1 day of injection seen by the co-localization of Cy3 (miR-126), Cy5 (dendrimer), isolectin (CNV blood vessel) and Iba1 (macrophage). The pattern of co-localization was maintained for up to 14 days. miR-126 also appeared to localize in the CNV target area for up to 7 days but the uptake pattern seemed more punctuated, correlating more with macrophage staining whereas there was broader uptake of D-miR126. Further, D-miR126 stayed in the target area for up to 14 days as detected by confocal microscopy whereas the majority of miR-126 was cleared by day 7.

Uptake of D-miR126 was confined to the CNV area and its surroundings at 24 h, similar to the distribution of free miR-126. However, the D-miR126 conjugate was retained at the target area for up to 14 days. At later time points, D-miR126 appeared to localize preferentially in macrophages with the majority of the dendrimer Cy5 signal and the miR-126 Cy3 signal co-localizing with Iba1 antibodies. Uptake of miR-126 was isolated to the CNV area and its immediate surrounding 24 h after intravitreal injection. Most of the free miRNA was cleared by day 7 as imaged through fluorescence microscopy. At 14 days, there were almost no remaining miRNA in the target area. The absence of Cy5 fluorescence corresponded to the lack of dendrimer in the free miRNA treatment group.

The fraction of signals co-localized within two stained cell populations was also analyzed (FIG. 14). Free miR-126 was gradually taken up by macrophages and endothelial cells over a period of 5 days. At 24 h post-injection, around 2% of the miR-126 was detected within macrophages as indicated by fraction colocalized with Iba1 signal and around 3% of the miR-126 was detected in the combined macrophage and endothelial population (stained with isolectin GS-IB4). The signals peaked at 5 days where ˜10% of the signals co-localized within macrophages and ˜15% within the combined macrophage/endothelial population.

In contrast, D-miR126 was rapidly taken up by resident macrophages within the CNV area with around ˜8% of the signals co-localizing with macrophage staining. Interestingly, only 2% of the signals are observed in the combined macrophage/endothelial population. However, by day 3, the signal distribution from Cy3 labeled miR-126 appear to migrate to distribute more evenly between the different cell populations. Around ˜6% of the signal localized with Iba-1 and a similar percentage with isolectin GS-IB4. The ratio of signals within Iba-1 positive cells and isolectin stained cells remained similar for the remaining time points. In addition, the level of miR-126 colocalized within the two cell populations remain relatively stable (˜10%) over the time course except for a dip on day 7. The co-localized signal between dendrimer (Cy5) and miR-126 (Cy3) was also examined as a measure of payload release in vivo. At 24 h post injection, about 50% of miR-126 was released from the dendrimer platform and the total release of miR-126 increased to about 80% by day 14.

### Discussion

MicroRNA is a powerful treatment option due to its ability to bind and degrade multiple targets implicated in the progression of a particular disease. However, this also means that delivery of miRNA to proper cells is crucial to avoid off-target effects and to optimize their efficacy. A dendrimer platform has been demonstrated to selectively target area of inflammation and angiogenesis and effectively deliver miRNA to treat choroidal neovascularization.

This platform utilizes generation 6 PAMAM dendrimers which have been shown to be biocompatible and possess longer circulation time. The surface was modified with environment-sensitive, disulfide linkers that could be used to attach nucleic acids and selectively release the payload in the intracellular compartment. The number of PDP linker moieties attached to the surface was higher than the 1:1 stoichiometric ratio of dendrimers to miRNA in the final compound due to steric hinderance considerations. Because dendrimer-miRNA conjugation chemistry involves two large biomolecules, lower conjugation efficiency was expected and therefore, more attachment sites were included to increase conjugate formation. In addition, miR-126 was added in excess to encourage binding to the dendrimer platform. After purification, gel electrophoresis and HPLC confirmed conjugate formation and the removal of unreacted nucleic acid. The combination of MALDI-TOF and gel electrophoresis provided an estimate of nucleic acid loading at 1:1 (dendrimer:miRNA).

In in vitro sink conditions, similar performances in D-miR126 and miR-126 were expected since cells can freely uptake the compounds without complex protein interactions, competing cell populations, and elimination mechanisms. In HMECs, no significant difference was observed in the performance of D-miR126 and miR-126 in reducing VEGF-A production. Similarly, in line with such expectations, reduction in TNFα mRNA levels were similar for D-miR126 and miR-126 treated BV2 cells. Interestingly, however, IL-1β levels appear to exhibit different dose responses dependent on the platform. For D-miR126 treated cells, a strong dose response is observed with a higher knockdown effect seen at 100 nM. In contrast, miR-126 treated cells exhibit an inverse response with a high knockdown effect seen at 10 nM, a magnitude lower than the most effective D-miR126 concentration.

This effect could be due to both the complex dose-dependent effect of miRNAs and by the slower release of D-miR126. First, theoretical models have attempted to decipher the complex interaction of miRNAs with their pool of targets and signaling pathways. In particular, miRNAs can preferentially impact different targets depending on their concentration, affinity to other targets, and the feedback from signaling pathways. As a result, dependent on the desired target, different doses of miRNA may be required to optimize its efficacy. This may partially explain the inverse relationship we observe in IL-1β mRNA production in BV2 cells.

In addition, dendrimer-conjugates have been shown to exhibit slower release profiles which restrict access of RISCs to the attached miRNA. In the case of D-miR126, release of the miRNA payload may be slow enough that only a fraction of miRNA was able to exhibit its effect over the assay time. As a result, increasing the treatment concentration of D-miR126 only partially increased the amount of released miR-126 in the cytosol, maintaining the range of optimal therapeutic concentration. Because the available concentration of cytosolic miR-126 was within the optimal concentration, the efficacy exhibited a dose-dependent response rather than an inverse response.

In tube formation assays, HMECs treated with either D-miR126 and miR-126 exhibited cell network disruption with lower doses of D-miR126 exhibiting higher efficacy at suppressing network formation. The increase in D-miR126 efficacy compared to free miR-126 may be attributed to the slower release mechanism which can maintain a more stable intracellular concentration of miR-126 when HMECs are stimulated by MATRIGEL® in the absence of miR-126 treatment.

Because of the complex interaction between miRNA concentration and target selectivity, the compounds were evaluated in laser induced CNV mouse models. First, the distribution of D-miR126 and miR-126 was determined through confocal microscopy and it was found that D-miR126 appeared to not only stay longer within the target CNV area but also distributed to both macrophage and endothelial cells, two cell subpopulations important for angiogenesis. This indicates that D-miR126 can influence both angiogenic and inflammatory responses of CNV formation and can do so over a longer time period, reducing the necessity for additional doses.

To assess the efficacy of CNV attenuation, choroids of treated and untreated mice were examined at day 7 and day 14. The area of D-miR126 treated mice significantly reduced CNV reduction at day 14 while miR-126 treated mice resulted in an insignificant decrease in area. The area decreases correlate well with decreases in VEGF-A, TNFα, and IL-1β levels as measured through either PCR or ELISA assay. In addition, co-localization measures of fluorescently labeled miR-126 and dendrimers, and stained macrophage and endothelial cells revealed differences in uptake kinetics and distribution characteristics between free miR-126 and D-miR126. D-miR126 appeared to achieve a higher intracellular concentration at earlier time points compared to free miR-126 and the payload is gradually released over time. This difference in uptake may enhance the therapeutic potential of miR-126 at early stages and prolong its efficacy at later time points.

### Conclusion

The flexibility of miRNA to target multiple proteins and pathways can be a powerful tool in treating previous untreatable diseases. However, harnessing its potential relies on effective delivery and dosing. A dendrimer platform has been established for the targeted delivery of miRNA to select cell populations. In addition, the effect of dendrimers on miRNA dosing has been characterized, and the effectiveness of dendrimer-miRNA conjugates in treating clinically relevant models has also been validated. The work presented constitutes a major step toward developing clinically translatable miRNA therapies.

### Example 4: Dendrimer Conjugate for Treatment of Macular Degeneration

Millions of elderly patients in developed countries face the risk of vision loss due to age-related macular degeneration (AMD) and approximately 10% of these patients will develop wet AMD. Wet AMD is a complex process in which choroidal neovascularization pushes blood vessels from the choroid through the Bruch's membrane to displace or destroy the retinal pigment epithelium (RPE). A prominent factor in the disease progression of wet AMD is the elevated expression of vascular endothelial growth factor (VEGF) in the eye which encourages the growth of new blood vessels. As a result, most current standards of care for wet AMD target VEGF directly through intravitreal injections of anti-VEGF antibodies such as aflibercept. However, a significant portion of patients (˜⅓) do not respond to these therapies and vision can still decline despite optimal patient adherence to the treatment regimen (D. Vogt, V. Deiters, T. R. Herold, S. R. Guenther, K. U. Kortuem, S. G. Priglinger, A. Wolf, and R. G. Schumann, Curr Eye Res, 2022, 1-8).

Other therapeutic modalities such as integrin binding peptides are being developed to halt the progression of wet AMD in patients. These peptide antagonists bind strongly to cell surface integrins such as αVβ3, α5β1, and α5β3 and inhibit the downstream signaling of these integrins. Specifically, these integrin antagonists reduce activation of ERK and PI3K/Akt pathways which in turn attenuate the expression of a variety of pro-inflammatory and pro-angiogenic cytokines such as VEGF-A, TNF-α, and IL1β. Luminate (or ALG-1001) is one such integrin binding peptide that has proven successful in halting neovascularization and is currently undergoing clinical trials for applications in AMD and in diabetic macular edema (DME). However, peptide-based antagonists suffer from a wide range of delivery challenges including rapid enzymatic degradation and renal clearance. Thus, these therapies are currently restricted to intravitreal injections not only limits their availability to patients in less developed countries but also carry risks of endophthalmitis, elevated intraocular pressures (IOP), and irritation.

### Materials and Methods

### Chemicals and Reagents

Hydroxyl-terminated, ethylenediamine-core PAMAM dendrimer (generation 6, pharmaceutical grade) in methanol solution was purchased from Dendritech (Midland, MI, USA). Prior to use, dendrimer solution was evaporated on a rotary evaporator. Dialysis membrane (MWCO 1 kDa) was purchased from Spectrum Chemicals (New Brunswick, NJ, USA). ALG-1001 and ALG-1001 modified with azide linker were purchased from Bio-Synthesis (Lewisville, TX, USA). Deuterated solvents (DMSO-d₆), methanol (CD₃OD), and water (D₂O) were purchased from Sigma-Aldrich. Proteinase K stock solution and Dulbecco's modified Eagle medium (DMEM, low glucose with L-glutamine) were purchased from ThermoFisher (Waltham, MA, USA). Human umbilical vein endothelial cells and the required media kit were purchased from Lonza (Basel, Switzerland). Phenol Red-free Matrigel was purchased from Corning (Tewksberry, MA, USA).

### Instrumentation

### Nuclear Magnetic Resonance

A Bruker 500-MHz spectrometer was used to obtain ¹H NMR spectra at ambient temperatures. Chemical shifts are reported in parts per million relative to tetramethylsilane which is used as an internal standard and the residual protic solvent peaks were used for chemical shifts calibration. DMSO-d6 (δ=2.50 ppm). The resonance multiplicity in the spectra is indicated as “s” (singlet), “d” (doublet), “t” (triplet), and “m” (multiplet). Broad resonances are expressed by “b.”

### High-Performance Liquid Chromatography

Waters HPLC (Milford, MA) equipped with 1525 binary pump, and in-line degasser AF, a 717 plus autosampler, and a 2998 photodiode array detector interfaced with Waters Empower software was used to determine the purities of compounds. The column was a Waters Symmetry C18 reversed-phase column with a particle size of 5 μm, length of 25 cm, and an internal diameter of 4.6. mm. The chromatograms were monitored at 210, 650, and 530 nm using the photodiode array (PDA) detector. The analysis was performed with a gradient flow starting at 95:5 (H₂O/ACN) increasing to 50:50 (H₂O/ACN) in 30 min and returning to 95:5 (H₂O/ACN) in 10 min at a flow rate of 1 ml/min.

### Synthesis of Dendrimer Conjugates

### Synthesis of Dendrimer-Hexyne

5-Hexynoic acid and DMAP were added to a solution of D-OH in anhydrous DMF and stirred at RT for 15 minutes. EDC·HCl was added to the resulting clear solutions in 3 equal portions and the solution was stirred overnight at room temperature. The reaction mixture was purified by dialysis through a 2 kDa MW cut-off cellulose dialysis membrane against DMF with solvent change at 8 h intervals. After 24 h, the mixture was dialyzed against water for 24 h with solvent change at 12 h intervals. The final aqueous solution was lyophilized to get the product as a white solid.

¹H NMR (500 MHz, DMSO) δ 8.06-7.79 (m, internal amide 510H), 4.73 (s, surface OH, 232H), 4.06 (s, ester linked, 22H), 3.40 (d, dendrimer —CH₂), 3.33 (d, dendrimer —CH₂), 3.18-3.11 (m, dendrimer —CH₂), 2.64 (s, dendrimer —CH₂), 2.43 (s, dendrimer —CH₂), 2.20 (s, dendrimer —CH₂), 1.68 (t, acetylene, 30H). Retention time: 19.58 min

### Synthesis of BOC-GABA-D-Hexyne

D-hexyne was dissolved in anhydrous DMF and BOC-GABA-OH and DMAP were added. The solution was stirred for 15 minutes before the addition of EDC·HCl in 3 equal, separate portions. The solution was stirred overnight, purified by dialysis, and lyophilized to get the product as a white solid.

¹H NMR (500 MHz, DMSO-d₆) δ 8.06-7.79 (m, internal amide 510H), 4.73 (s, surface OH, 199H), 4.06 (s, ester linked, 40H), 3.40 (d, dendrimer —CH₂), 3.33 (d, dendrimer —CH₂), 3.18-3.11 (m, dendrimer —CH₂), 2.64 (s, dendrimer —CH₂), 2.43 (s, dendrimer —CH₂), 2.20 (s, dendrimer —CH₂), 1.68 (t, acetylene, 27H), 1.36 (s, BOC, 68H).

### Synthesis of GABA-D-Hexyne

Deprotection of BOC-GABA-D-Hexyne was performed under anhydrous conditions. The compound was placed in a round bottom flask and anhydrous DCM was added under nitrogen atmosphere. The solution was constantly stirred and sonicated to form a cloudy, sticky suspension. TFA was then added to the suspension in a 4:1 ratio (DCM:TFA) and the solution was stirred overnight. DCM was then evaporated using a rotary evaporator. TFA was removed by repeatedly diluting the reaction mixture with methanol and evaporating the resultant solution. The product was then placed under high vacuum for 3 hours and used without further purification.

### Synthesis of Cy5-D-Hexyne

GABA-D-Hexyne was dissolved in anhydrous DMF, followed by DIPEA, and finally the addition of Cy5 NHS ester. The reaction was stirred overnight, and the reaction mixture was dialyzed through a 2 kDa membrane against DMF for 24 h. The mixture was then dialyzed against water for an additional 24 h and lyophilized to obtain a solid, blue product.

¹H NMR (500 MHz, DMSO-d₆) δ 8.06-7.79 (m, internal amide 510H), 7.35 (m, Cy5 H), 7.25 (m, Cy5 H), 7.05 (m, Cy5 H), 6.6 (m, Cy5 H), 6.3 (m, Cy5 H), 4.73 (s, surface OH, 199H), 4.06 (s, ester linked, 40H), 3.40 (d, dendrimer —CH₂), 3.33 (d, dendrimer —CH₂), 3.18-3.11 (m, dendrimer —CH₂), 2.64 (s, dendrimer —CH₂), 2.43 (s, dendrimer —CH₂), 2.20 (s, dendrimer —CH₂), 1.68 (t, acetylene, 27H). Retention time: 18.01 min

### Synthesis of D-ALG1001 and Cy5-D-ALG1001

ALG-1001 was dissolved in ultrapure water and added to an aqueous solution of D-Hexyne for unlabeled conjugates. ALG-1001-Cy3 was dissolved in ultrapure water and added to an aqueous solution of Cy5-D-Hexyne for dual-labeled conjugates. A solution of copper sulfate was added and the solution stirred for 10 minutes RT before adding sodium ascorbate. For purification, both reactions were left at room temperature overnight then dialyzed against water for 24 h. Each solution was lyophilized to obtain a powder product.

¹H NMR (500 MHz, DMSO-d₆) of D-ALG: 8.12-7.78 (m, internal amide H), 4.45-4.06 (m, peptide a carbon), 4.00 (s, ester linked, 37H), 3.78 (m, polyethylene glycol H), 3.40 (d, dendrimer —CH₂), 3.33 (d, dendrimer —CH₂), 3.18-3.11 (m, dendrimer —CH₂), 2.64 (s, dendrimer —CH₂), 1.64-1.59 (m, GABA linker —CH2 and peptide side chain). Retention time: 16.60 min ¹H NMR (500 MHz, DMSO-d₆) of Cy5-D-ALG-Cy3: 8.12-7.78 (m, internal amide H), 7.01 (m, aromatic 5H), 6.59 (s, GABA amide H, 10H), 4.73 (s, surface OH, 233H), 4.06 (s, ester linked, 16H), 3.40 (d, dendrimer —CH₂), 3.33 (d, dendrimer —CH₂), 3.18-3.11 (m, dendrimer —CH₂), 2.64 (s, dendrimer —CH₂), 1.64-1.59 (m, GABA linker —CH2, 6H), 1.36 (s, Boc group, 20H). Retention time: 17.99 min

### In Vitro Stability Under Enzymatic Degradation

Proteinase K stock solution was purchased from ThermoFisher and used as is. ALG-1001 and D-ALG solutions were prepared at a concentration of 2 mg/mL, and proteinase K was added to each solution such that the final concentration of proteinase K is 2 mg/mL. The mixture was incubated at 37° C. and at set time points, 100 μL of the mixture was removed and analyzed through HPLC. To determine compound degradation, integration of peaks at elution times associated with ALG-1001 and D-ALG were used and normalized to the injected analyte peak at the 0 h time point.

### Cell Culture

HUVEC cells were obtained from Lonza and cultured in EGM-2 endothelial cell growth medium (Lonza). Murine macrophages (RAW264.7) between passages 5-9 were cultured in Dulbecco's modified Eagle's medium (DMEM, Life technologies, Grand Island, NY) supplemented with 10% fetal bovine serum (Invitrogen Corp., Carlsbad, CA) and 1% penicillin/streptomycin (Invitrogen Corp., Carlsbad, CA). All cells were maintained at 37° C. and 5% CO₂ with a humidified incubator.

### In Vitro Evaluation of D-ALG Efficacy

### Vessel Formation Assay

96 well plates were coated with 75 μL of MATRIGEL® and left at room temperature for 15 minutes before placing into 37° C. incubators for an additional 30 minutes. D-ALG1001 was dissolved at twice the desired concentration and 50 μL of the drug solution was added to the wells. HUVEC cells were then added to each well at a density of 70,000 cells/cm². Live-cell images were taken at 12 h for analysis.

### Wound Healing Assay

HUVEC cells were seeded at a density of 5×10⁴ cells per well in 24-well plates and set aside for at least 72 h for a uniform monolayer of cells to form. Cells were treated with D-ALG1001 and ALG-1001 for 24 h. A centimeter long scratch was introduced to the cell monolayer with the tip of a 200 μL pipette tip. Images were taken on a Nikon.

### VEGF Activation for Western Blotting and PCR

HUVEC cells were seeded at a density of 5×10⁴ cells per well in 24-well plates 24 h before treatment. Cells were treated with D-ALG1001 and ALG-1001 for 24 h before activation with VEGF for 5 minutes. Cells were collected and lysed for Western blotting using T-Per buffer (Thermofisher) supplemented with PHOSSTOP® and proteinase inhibitor cocktail. Cells were lysed with TRIZOL® and processed as previously described for qPCR analysis.

### In Vitro Inflammation Model

RAW264.7 cells were seeded at a density of 1×10⁵ cells per well in 12-well plates 48 h before treatment. Cells were incubated with D-ALG1001 and ALG-1001 for 24 h, the treatment media was aspirated then LPS at a concentration of 10,000 Endotoxin Units/mL was added to stimulate inflammatory response. Samples were collected 3 h after LPS stimulation for qPCR analysis.

### In Vivo Evaluation of D-ALG at Attenuating Choroidal Neovascularization

### In Vivo Laser CNV Rat Models

All animal procedures were approved by the Johns Hopkins Animal Care and Use Committee. Brown Norway rats between 8-12 weeks old were obtained and housed at constant temperature and humidity (20±1° C., 50±5% humidity). Animals were anesthetized through intraperitoneal injections of a ketamine/xylazine cocktail (ketamine 50 mg/kg and xylazine 10 mg/kg). Pupils are dilated with topical 2.5% Phenylephrine Hydrochloride solution followed by 0.5% Tetracaine Hydrochloride solution. To induce CNV formation, four equally spaced lesions will be created in the Bruch's membrane with a built-in laser system on the Micron III SLO. Laser power was set to 240-250 W with duration of 70 ms. Gonio ophthalmic solution was applied post-operatively to prevent eyes from drying and forming cataracts.

On the day of CNV induction (Day 0), D-ALG1001 and ALG1001 was administered intraperitoneally at a dose of 150 μg on a peptide basis. Subsequent doses were administered every 4 days. At day 7 and 14, mice were sacrificed and enucleated. Eyes used for CNV image were fixed in 10% formalin for 1 h. Eyes used for qPCR, ELISA, and western blotting were immediately stored in −80° C. until use.

Tissue Preparation for Western and qPCR

In brief, 500 μL of T-Per supplemented with PHOSSTOP® and proteinase inhibitor cocktail was added to tubes containing dissected choroid. A scoop of 1.6 mm steel homogenization beads was added to each sample and then placed in TISSUELYSER® LT (Qiagen) at an oscillation frequency of 50/s for 15 minutes. For qPCR, 200 μL of TRIZOL® was added to tubes containing choroid and a scoop of 1.6 mm steel homogenization beads was added. The samples were placed in TISSUELYSER® LT at an oscillation frequency of 50/s for 15 minutes.

Pathway Activation Analysis with Western Blot and ELISA

Concentration of protein was determined with the BCA protein assay kit (Thermo Scientific, Rockford, IL). Equal amount of protein was denatured and resolved on 4-15% TGX gels (Bio-Rad, Hercules, CA). Gels were transferred to nitrocellulose membranes, blocked with 3% Bovine Serum Albumin (“BSA”) at RT for 1 h, and probed for GAPDH, FAK, pFAK, MAPK, and pMAPK at 4° C. overnight. Membranes were washed thrice before incubation with HRP-conjugated secondary antibodies followed by incubation with chemiluminescent substrate for visualization.

For protein extracted from tissues, total FAK and FAK (Phospho) [pY397] ELISA kits (Invitrogen) were used to quantify the level of protein expression and the measured protein expression was normalized to the total protein amount of each sample as determined from BCA assays.

qPCR Analysis

100 μL of chloroform was added to the Trizol suspension and the aqueous phase was separated with a centrifuge at 10K RPM at 4° C. for 15 minutes. 400 μL of 2-propanol was added to the aqueous solution and spun again to pellet the RNA. The RNA was washed with 70% ethanol solution, pelleted again, and re-suspended in DEPC (ultrapure treated to inactivated enzymes) water.

To convert RNA to complementary DNA, 2 μg of RNA was converted using a High-Capacity cDNA Reverse Transcription Kit (Applied Biosystems, Foster City, CA). Samples were analyzed using STEPONE PLUS® real-time PCR system (Applied Biosystems) with SYBR Green reagent (ThermoFisher Scientific). Relative expression was quantified with ΔΔCt calculations normalized to controls. Primers for GAPDH was obtained from Bio-Rad Laboratories (Hercules, CA). Primers were purchased from Integrated DNA Technologies (Coralville, IA). Primers for TNFα were:

forward:
(SEQ ID NO: 6)
CCA GTG TGG GAA GCT GTC TT;
and
reverse:
(SEQ ID NO: 7)
AAG CAA AAG AGG AGG CAA CA.

Primers for IL1β were:

forward:
(SEQ ID NO: 8)
AGC TTC AAA TCT CGA AGC AG;
and
reverse:
(SEQ ID NO: 9)
TGT CCT CAT CCT GGA AGG TC.

### Biodistribution

For biodistribution studies, a dose of Cy5-D-ALG1001-Cy3 and ALG-1001-Cy3 at 150 μg/100 μL was administered to each animal intraperitoneally on the day of CNV induction (Day 0). Animals were sacrificed at 1-, 2-, 3-, and 4-day time points and enucleated.

### Imaging

After fixation, the posterior segment of the eye was dissected out and the retina separated from the choroid. Tissues stained with FITC-labeled isolectin (GS IB₄) (Life Technologies, Eugene, OR) for blood vessels and monocytes. Eyes were mounted by introducing four radial relaxation cuts. Samples for biodistribution were imaged under a confocal 710 microscope (Carl Zeiss, Oberkochen, Germany). Samples for CNV quantification were imaged with an Axiovert phase contrast microscope. All images were processed with ImageJ.

### Statistical Analysis

Data were presented as means±SEM and analyses were performed in GraphPad Prism (version 9; La Jolla, CA). Treatment groups across time point or doses were analyzed through analysis of variance (ANOVA) tests. Significant differences among single groups were determined with Student's t tests: *P<0.05, **P<0.01 and ***P<0.001.

### Results

### Synthesis and Characterization of D-ALG1001 Intermediates and Conjugates

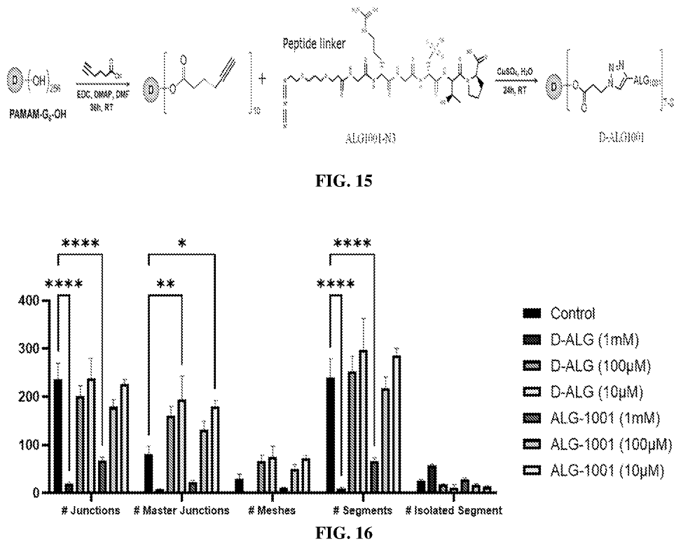

A high-yield click reaction was used to efficiently attach the ALG-1001 peptide to the dendrimer platform under mild conditions that is amenable to preserving the integrity and activity of the peptide (FIG. 15). First, the dendrimer surface was modified with hexynoic acid linker and modification was confirmed by ¹HNMR with the presence of 20 protons at 4.0 ppm and 1.7 ppm. The dendrimer surface was minimally modified to preserve its near neutral charge and its inherent ability to penetrate tissues.

For biodistribution studies, the dendrimer surface is further modified with GABA-Boc linkers. The presence of additional protons at 4.0 ppm, 1.7 ppm, and 1.2 ppm confirms the modification. The resulting intermediate was deprotected and the free amine was used to couple with Cy5 ester to obtain fluorescently labeled dendrimer.

ALG-1001 peptide was purchased with a short polyethylene glycol (PEG) azide linker attached to the C terminus and used without further preparation. For biodistribution studies, the N terminus of the peptide was also modified with Cy3 fluorophore for tracking. Copper (I) catalyzed alkyne-azide click (CuAAC) reaction was used to attach ALG-1001 to dendrimers and ¹HNMR spectra confirmed the attachment of ALG-1001 by showing the presence of the peptide protons.

### In Vitro Stability Under Enzymatic Degradation

Co-incubation of ALG-1001 and proteinase K, a broad acting protease, resulted in a rapid degradation of ALG-1001 with about 50% degraded at 30 minutes and 90% at 90 minutes. HPLC chromatographs of D-ALG and ALG-1001 after co-incubation with proteinase K show decrease in AUC of peak associated with the free ALG-1001 peptide. The trace of D-ALG show negligible decrease in AUC. Curve plotting amount degraded is shown by normalizing peaks of analyte taken at set time points to the starting peak obtained at 0 minutes. About 90% of ALG-1001 was degraded by 90 minutes whereas only 10% of D-ALG was degraded. On the other hand, dendrimer conjugation of the ALG-1001 peptide conferred resistance to enzymatic degradation, possibly due to steric hinderance from the dendrimer carrier. Only around 10% of D-ALG was degraded at 90 minutes. This resistance toward enzymatic degradation even with an extraordinarily high enzyme concentration suggests that dendrimer conjugation may increase the circulation time of intact peptides in vivo by helping it escape degradation pathways.

### In Vitro Vessel Formation Assay

To find the effective dose in a physiologically relevant model, an in vitro model of blood vessel formation was utilized. HUVEC cells were treated with a gradient concentration of D-ALG and ALG-1001 for 24 h at three different magnitudes of doses. The cells were then seeded on MATRIGEL® after which cells naturally migrate to form blood vessel-like tubule structures. The images were analyzed with Angiogenesis Analyzer plug-in on ImageJ to extract relevant metrics that measure the connectivity and integrity of tube networks (FIG. 16).

Cells treated with 1 mM of D-ALG and ALG-1001 exhibit increased disruption in tube formation with more isolated segments and smaller area or meshes enclosed by tubes. The data indicate that dendrimer conjugation increases the efficacy of ALG-1001 at disrupting network formation. HUVECs treated with 1 mM of D-ALG had fewer points of intersection between vessels (junctions), fewer connected segments, and more isolated segments compared to those treated with 1 mM of ALG-1001.

### Wound Healing Assay

In addition to cell morphological and motility changes measured by tube formation assays, the proliferative activity of HUVECs treated with D-ALG and ALG-1001 was assessed in a wound healing assay. Monolayers of HUVECs were pretreated with D-ALG and ALG-1001 followed by scratching the monolayer with a pipette tip. Images were taken at the time of the wound and at 24 h after injury. Untreated cells managed to heal up to 80% of the initial damage after 24 h while cells treated with D-ALG and ALG-1001 healed up to 50% of the initial wound area. Further, cells treated with a high dose of D-ALG at 1 mM only recovered 20% of the initial damage, suggesting a reduced ability of HUVECs regenerate. The trend indicates that treatment with D-ALG is more efficacious than the free peptide.

### Western Blot on Endothelial Cell Activation

To elucidate the mechanism through which D-ALG and ALG-1001 influence angiogenic function, cells were pre-treated with D-ALG and ALG-1001 for 24 h. The cells were then stimulated with a high dose of exogenous VEGF for 5 min after which the samples were collected for western blotting. The samples were probed for expression of total FAK, phospho-FAK (Y397), ERK1/2, phospho-ERK1/2, and Cyclophilin B (CycB) with CycB acting as the internal control.

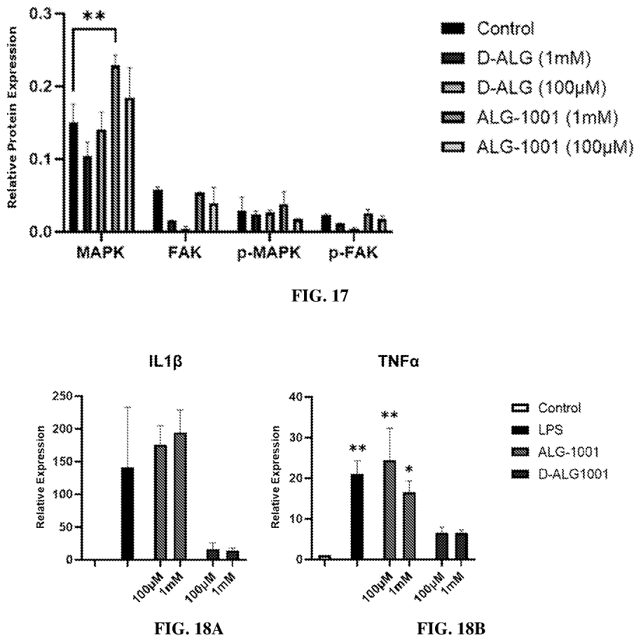

Compared to untreated VEGF stimulated controls, the results show that cells treated with D-ALG and ALG-1001 expressed lower levels of total FAK and phosphor-FAK. At high doses of 1 mM on a peptide basis, D-ALG and ALG-1001 reduced phosphorylation of phospho-ERK1/2 by ˜60% and phospho-FAK by 20% compared to VEGF stimulated controls. Both ERK1/2 and FAK pathways are important players in angiogenesis and their activation promotes endothelial cell proliferation and migration (FIG. 17). The trend in the reduction in phosphorylated proteins suggests attenuation in the activation of these networks in response to VEGF.

In addition to examining pathway activation, the effects of D-ALG and ALG-1001 were also studied on VEGF-A expression by endothelial cells. Cells treated with VEGF-A significantly increased the production of VEGF-A compared to unstimulated controls. Cells co-incubated with VEGF-A and either ALG-1001 or D-ALG did not have statistically significant increases in VEGF production compared to the control, suggesting a slight attenuation in endothelial activation in response to VEGF.

### Attenuation of Inflammatory Response in Murine Microglia

Another important player in angiogenesis is macrophages and microglia which produce pro-inflammatory and pro-angiogenic cytokines during the angiogenic process. To elucidate the effect of D-ALG and ALG-1001 on macrophage activation, RAW264.7 cells were pre-treated with a high and low dose of D-ALG and ALG-1001 24 h prior to LPS stimulation. The treatment media was first aspirated to simulate the transient nature of in vivo delivery and cells were activated with LPS for 3 h.

Cells activated with LPS displayed a robust increase in the expression of pro-inflammatory cytokines IL1β and TNFα as detected through qPCR when compared to unstimulated controls (FIGS. 18A-18B). With D-ALG treatment, the expression of IL1β was reduced by ˜90% and TNFα by 80% in both the low (100 μM) and high dose (1 mM). Strikingly, treating with D-ALG brings the level of TNFα to a level not statistically different than the untreated controls. In comparison, ALG-1001 reduced the expression of TNFα by 20% only at the highest dose and no impact on IL1β production was observed with ALG-1001 treatment alone. This difference may be due to two potential differences: (1) the dendrimer platform is demonstrated to be much more efficient at delivering therapeutics to activated macrophages compared to free drugs; (2) attaching multiple ligands on the dendrimer surface allows for multivalency effects, increasing the interaction of the peptide and surface integrin.

### Biodistribution of Systemically Administered ALG-1001 and D-ALG

Fluorescently labeled Cy3-ALG-1001 peptide and dual labeled Cy5-dendrimer-ALG-Cy3 were systemically injected on day 0 (same day as CNV induction) and choroidal tissues were collected at set time points. Confocal microscopy was used to monitor the presence of ALG-1001 peptide (Cy3), dendrimer carrier (Cy5), CNV formation (Isolectin), and macrophages (Iba1). Within the first 24 h of systemic administration, free ALG-1001 peptide reached the CNV area and remain there for up to 2 days as seen from detected Cy3 signals. In comparison, D-ALG was able to not only reach the CNV area within 24 h after systemic injection but also remain in the target area up to 4 days post administration from the co-localized presence of both Cy5 and Cy3 signals. The prolonged residence time indicates that dendrimer conjugation allowed the target area itself to serve as a drug depot, prolonging the efficacy of the peptide therapeutic.

### In Vivo Attenuation of CNV Formation

A Micron III SLO scope and laser attachment was used to induce CNV formation in mice. The laser CNV model was chosen due to its consistency in creating CNV and the progression of the CNV process. Whether dendrimer conjugation impeded the peptide from attenuating CNV was assessed by first injecting ALG-1001 and D-ALG intravitreally after CNV induction. The eyes were then collected on day 7 and CNV area quantified. Both D-ALG and ALG-1001 significantly inhibited the formation of CNV when administered intravitreally.

The protection and targeting of D-ALG allowed for less invasive administration routes. Laser was used to induce CNV at day 0 and the first dose (150 μg peptide basis) of ALG-1001 and D-ALG was administered intraperitoneally. The animals were dosed once every 4 days at 150 μg peptide basis. Choroidal flatmount of eyes at day 7 and 14 were imaged and the CNV area was calculated using ImageJ.

In untreated control animals, the CNV area reached a size of about 13,000 μm² by day 7 and about 14,000 μm² by day 14 after CNV induction (FIGS. 19A-19B). Systemic injection of ALG-1001 produced robust attenuation of CNV formation (˜60% reduction) on day 7 but CNV area recovered at day 14. In comparison, systemic administration of D-ALG suppressed the formation of CNV by ˜50% on day 7 and day 14, with day 14 reaching statistical significance. This improvement in CNV reduction and its sustained effect is likely attributed to the ability of D-ALG to protect the peptide payload and lengthen the residence time at the target CNV area.

### Systemically Administered D-ALG Attenuates FAK and ERK Activation

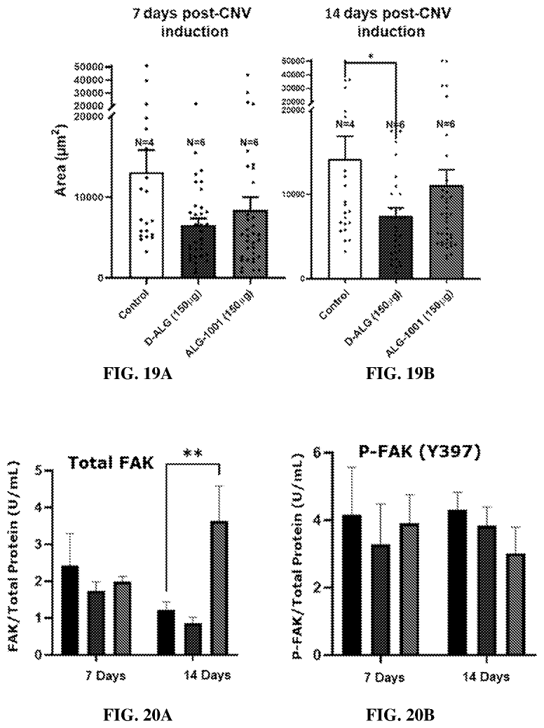

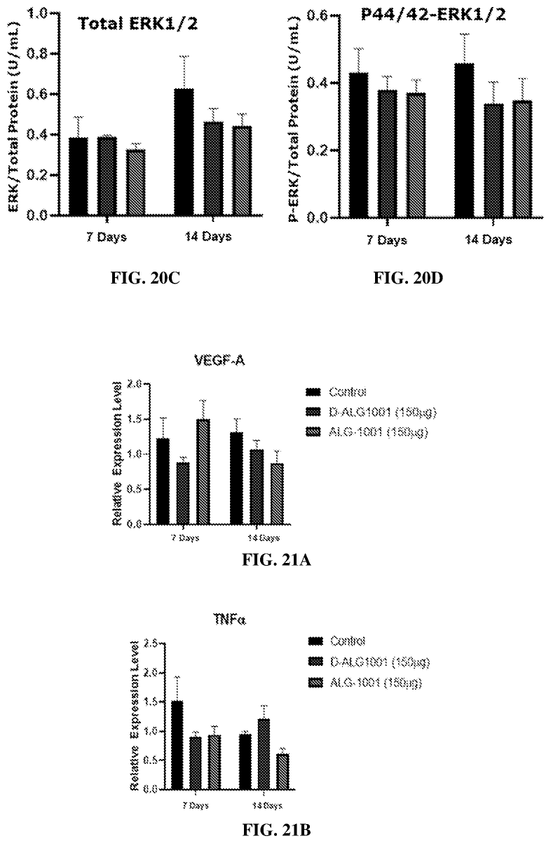

To evaluate activation of FAK and ERK pathways, eyes were enucleated at set time points and the choroidal tissue dissected out. Tissues were submerged in a mixture of T-per, proteinase inhibitor, and PhosStop with stainless steel homogenization beads and homogenized to produce protein extracts. Total FAK, total ERK, p-FAK (Y397), and p-44/42 ERK ELISA kits were used to quantify the amount of total and phosphorylated proteins in FAK and ERK pathways. In animals treated with D-ALG, a trend in the reduction of total FAK and p-FAK protein was observed in both 7 and 14 days compared to untreated controls, suggesting prolonged attenuation of the FAK pathway (FIGS. 20A-20B). Treatment of ALG-1001 also resulted in reduction of p-FAK across both time points. However, the level of total FAK protein in ALG-1001 treated animals was elevated at day 14. Both D-ALG and ALG-1001 treated animals resulted in a similar trend in the decrease of p44/42 ERK production while total ERK remained relatively constant at day 7 across treatment groups (FIGS. 20C-20D). On day 14, there is a slight reduction of total ERK in D-ALG and ALG-1001 treated animals compared to untreated animals.

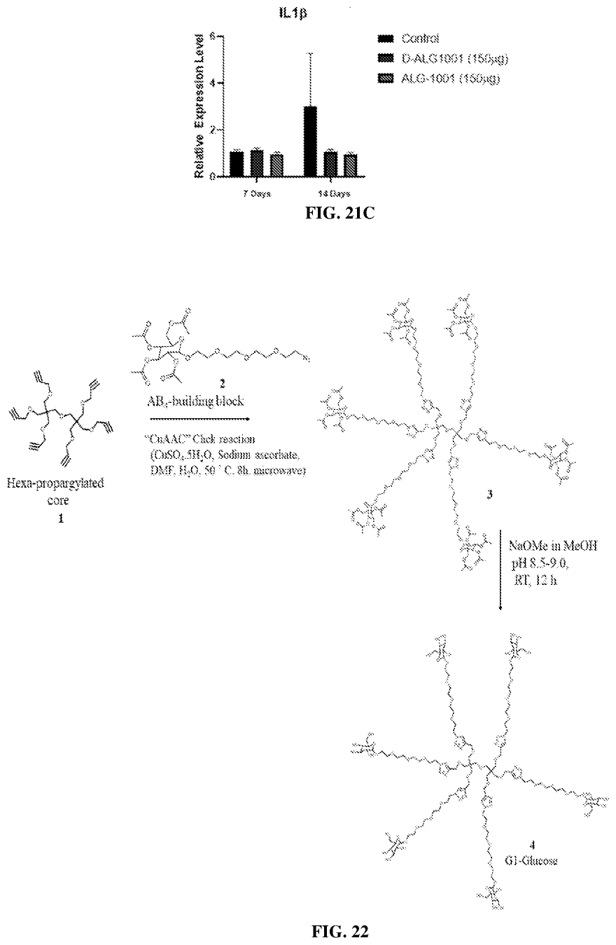

To compare the expression of pro-inflammatory and pro-angiogenic cytokines, RNA was extracted, and qPCR was performed to determine the relative expression of VEGF-A, TNFα, and IL1β (FIGS. 21A-21C). In D-ALG treated animals, VEGF-A production was reduced across both 7 and 14 days whereas ALG-1001 treated animals expressed lower VEGF-A production at 14 days only. Comparing trends in inflammatory cytokines, D-ALG produced lower TNFα level at 7 days only while ALG-1001 treated animals had lower TNFα levels across both time points. The trend of IL1β expression was only reduced at day 14 for ALG-1001 and D-ALG treated animals.

### Discussion

Dendrimer conjugation of ALG-1001 peptide was accomplished through highly efficient copper assisted click reaction under mild conditions and characterized through HPLC and NMR. Through integration of NMR peaks, it was calculated that 6-7 peptides were attached per dendrimer carrier. An increased resistance to enzymatic degradation was observed with dendrimer conjugation in vitro after incubation with a broad acting proteinase.

At high, equivalent doses, D-ALG conjugates inhibited vessel formation an order of magnitude better than the free peptide. In addition, D-ALG1001 attenuated not only the activation of the FAK pathway in endothelial cells but also the expression of pro-inflammatory cytokines in LPS stimulated murine macrophages. It was hypothesized that by attaching multiple peptide moieties on a single dendrimer may allow the integrin clusters to be engaged more effectively, enhancing the anti-angiogenic and anti-inflammatory activity of ALG-1001 through this multivalency effect.

When systemically administered in vivo, differences in the distribution and bioavailability of D-ALG and free ALG-1001 peptide can be better elucidated. Fluorescently labeled D-ALG was detectable in the CNV area up to 4 days after intraperitoneal injection; on the other hand, ALG-1001 signal was undetectable after day 2. The increased residence time at the target area allowed for less frequent injections for D-ALG. Intraperitoneal injection of 150 μg of D-ALG (on a peptide basis) every four days resulted in a 50% reduction in CNV area whereas free ALG-1001 peptide reduced CNV formation by 40% on day 7 and loses potency at later time point (20% CNV reduction on day 14). In comparison, continuous systemic delivery of JSM6427, a small molecule α5β1 antagonist, using an implanted pump resulted in a 40% CNV area reduction (N. Umeda, et al., Mol. Pharmacol., 2006, 69, 1820-1828). Similarly, in separate studies by Das et al and Toriyama et al. exploring Å6 peptide and CGRP peptides respectively, daily injections were necessary to reduce CNV area by 30% (H. J. Koh, et al., Invest. Ophthalmol. Vis. Sci., 2004, 45, 635-640; Y. Toriyama, et al., Am. J. Pathol., 2015, 185, 1783-1794).

The data demonstrates that dendrimer conjugation can not only deliver biologics intact to the target area after systemic administration but also increase its residence time and efficacy. As a result, a less stringent dosing schedule is needed to effectively control CNV formation. The inherent ability of the dendrimer to be taken up by reactive macrophages and microglia allowed for a higher local concentration to be achieved while the rapid clearance of dendrimer conjugates from the blood reduced unnecessary exposure in non-targeted cells and tissues. Dendrimer conjugation to ALG-1001 peptide preserves the activity of the peptide, increases its stability, prolongs its residence time in the target tissue, and offers systemic administration as an alternative route to intravitreal injections, thereby expanding the availability of the therapy worldwide.

### Example 5: Synthesis of Glutamine Dendrimer Conjugates

FIG. 22 shows the synthesis of G1-Glucose. Stepwise synthesis of G1-glucose; the hexapropagylated core 1, was treated with AB4 building block (β-Glucose-PEG₄-azide), 2 under clasical click regents (CuAAC click reaction), catalytic amount of copper sulfate pentahydrate (CuSO₄·5H₂O) and sodium ascorbate in DMF:H₂O (1:1) to produce G1-glucose-24-OAc, 3. Then compound 3 was treated under typical Zemplén conditions (to remove acetate groups) to obtain the desired product 4 (G1-glucose).

FIG. 23 shows the synthesis of Glu-G2 dendrimer. Stepwise synthesis of G2-glucose; G1-glucose dendrimer 4 was treated with sodium hydride (60% dispersion in mineral oil) for 15 min at 0° C. and then treated with Propargyl bromide (80% w/w solution in toluene). The reaction was stirred at room temperature for 8 h to form compound 5. The compound 5 next treated with AB4 building block (β-Glucose-PEG₄-azide), 2 under clasical click regents (CuAAC click reaction), catalytic amount of copper sulfate pentahydrate (CuSO₄·5H₂O) and sodium ascorbate in DMF:H₂O (1:1) to produce G2-glucose-96-OAc, 6. And then compound 6 was reacted under typical Zemplén conditions to obtain the desired product 7 (G2-glucose).

FIG. 24 shows the synthesis of Cy5-Glu-G2-PEG₄-SPDP. The Glu-G2 dendrimer was treated with NaH and propargyl bromide and the resulted product, 2 was further reacted with N₃-PEG₃-amine, 3 using CUAAC click condition to form compound 4. The product, 4 was labeled with Cy5 fluorophore and the resulted intermediate, 5 was conjugated with SPDP to obtain functionalized Cy5-Glu-G2-PEG₄-SPDP, 6. The subscripted numbers in the formulas indicate the number of attachments per dendrimer.

FIG. 25 shows the synthesis of Cy5-Glu-G2-siRNA conjugate. The siRNA, 7 was activated by reducing dithiol group using DTT and the resulted product, 8 was reacted with activated Cy5-Glu-G2-PEG₄-SPDP, 6 to obtain the final product, Cy5-Glu-G2-siRNA, 9.

Uptake of Cy5-Glu-G2-siGFP into neuronal cells was confirmed by confocal microscopy images of Cy5-Glu-G2-siGFP cell uptake into neuronal cells. At 24 h post treatment, the Cy5-Glu-G2-siRNA dendrimer is colocalized within the cells.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising dendrimers covalently conjugated to one or more functional nucleic acids, optionally via one or more spacers,
wherein the functional nucleic acids are conjugated to less than 50% of the total terminal groups on the surface of the dendrimer prior to conjugation.

2. The composition of claim 1, wherein the one or more functional nucleic acids inhibit the transcription, translation, or function of a target gene.

3. The composition of claim 1, wherein the one or more functional nucleic acids are selected from the group consisting of antisense molecules, small interfering RNAs (siRNAs), microRNAs (miRNA), aptamers, ribozymes, triplex forming molecules, and external guide sequences.

4. The composition of claim 1, wherein the one or more functional nucleic acids comprise siRNA or miRNA.

5. The composition of claim 4, wherein the miRNA is miR-126.

6. The composition of claim 1, wherein the dendrimers are generation 2, generation 3, generation 4, generation 5, generation 6, generation 7, or generation 8 dendrimers.

7. The composition of claim 1, wherein the dendrimers are poly(amidoamine) (PAMAM) dendrimers or glucose dendrimers, with between greater than 40 and 100% of the surface groups being hydroxylated or conjugated to glucose monosaccharides.

8. The composition of claim 1, wherein the dendrimers are hydroxyl-terminated PAMAM dendrimers.

9. The composition of claim 1, wherein the dendrimers are glucose dendrimers made of glucose and ethylene glycol building blocks, with greater than 10 surface glucose moieties.

10. The composition of claim 1, wherein the dendrimers are covalently conjugated to the one or more functional nucleic acids via one or more spacers.

11. The composition of claim 1, wherein one or more spacers are selected from the group consisting of N-Succinimidyl 3-(2-pyridyldithio)-propionate (SPDP), glutathione, gamma-aminobutyric acid (GABA), polyethylene glycol (PEG), and combinations thereof.

12. The composition of claim 1, wherein the dendrimers are covalently conjugated to the one or more functional nucleic acids via disulfide bonds.

13. The composition of claim 1, wherein the dendrimers are further conjugated to one or more additional therapeutic, prophylactic, and/or diagnostic agents.

14. A composition comprising one of the following structures: 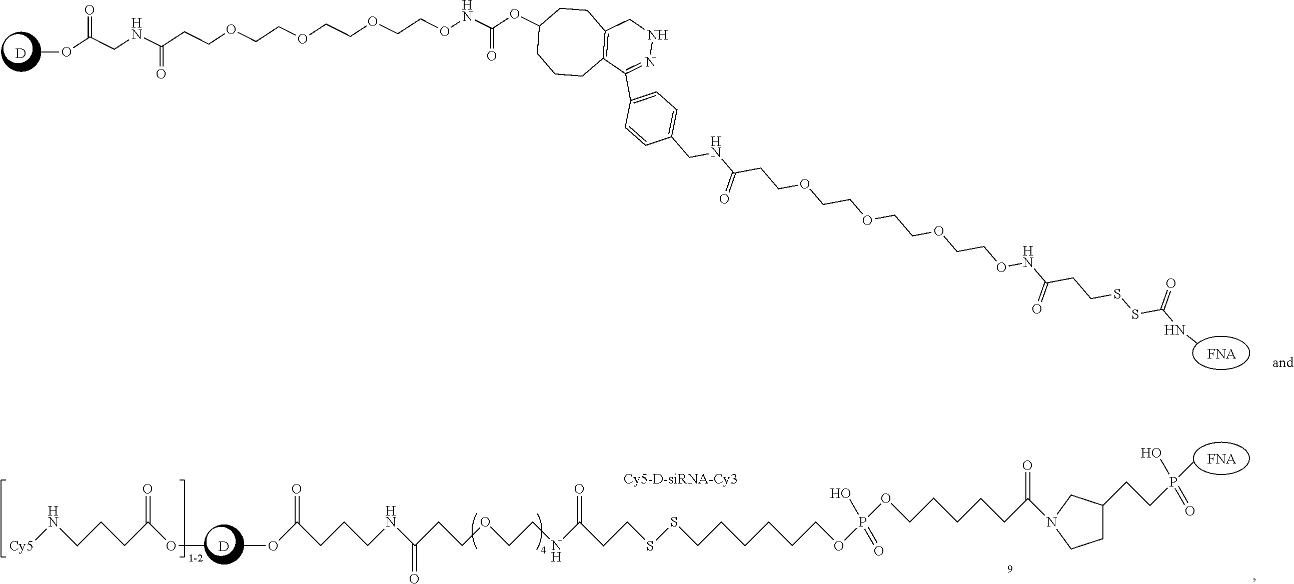
wherein the circle denoted with D is a hydroxyl-terminated dendrimer and the oval denoted with FNA is a functional nucleic acid.

15. A pharmaceutical composition comprising the composition of claim 1 and one or more pharmaceutically acceptable excipients.

16. The pharmaceutical composition of claim 15 formulated for parenteral or oral administration.

17. The pharmaceutical composition of claim 15 formulated in a form selected from the group consisting of hydrogels, nanoparticle or microparticles, suspensions, powders, tablets, capsules, and solutions.

18. A method for treating one or more symptoms of cancer, infectious disease, proliferative disease, or inflammation in a subject in need thereof comprising administering to the subject an effective amount of the composition of claim 1 to alleviate one or more symptoms of the cancer, infectious disease, proliferative disease, or inflammation.

19. The method of claim 18, wherein the inflammation is associated with one or more diseases, conditions, and/or injuries of the eye, the brain, and/or the nervous system (CNS).

20. The method of claim 19, wherein the one or more diseases, conditions, and/or injuries of the eye, the brain and/or the CNS are diseases, conditions, and injuries associated with activated microglia and astrocytes or injured, diseased, and/or hyperactive neurons, ganglion cells and other neuronal cells in the brain and the eye.

21. The method of claim 19, wherein the one or more diseases, conditions, and/or injuries of the eye is choroid neovascularization, and the functional nucleic acid is a miRNA specific for vascular endothelial growth factor (VEGF).

22. The method of claim 21, wherein the miRNA is miR-126.

23. The method of claim 19, wherein the one or more diseases, conditions, and/or injuries of the eye is macular degeneration.

24. The method of claim 19, wherein the composition is administered directly into the eye.

25. The method of claim 24, wherein the composition is administered by intravitreal injection.

26. The method of claim 18, wherein the cancer is selected from the group consisting of breast cancer, cervical cancer, ovarian cancer, uterine cancer, pancreatic cancer, skin cancer, multiple myeloma, prostate cancer, testicular germ cell tumor, brain cancer, oral cancer, esophagus cancer, lung cancer, liver cancer, renal cell cancer, colorectal cancer, duodenal cancer, gastric cancer, and colon cancer.

27. The method of claim 18, wherein the effective amount is effective to reduce tumor size or inhibit tumor growth.

28. The method of claim 18, wherein the composition is administered orally or parenterally.

29. The method of claim 18, wherein the composition is administered intravenously.

30. The method of claim 18, wherein the composition is administered at a time selected from the group consisting of once every day, once every other day, once every three days, once a week, once every 10 days, once every two weeks, once every three weeks and once every month.

31. The method of claim 18, wherein the composition is administered once every two weeks, or less frequently.

32. The method of claim 18, wherein the amount of the functional nucleic acid effective to treat the disease or disorder is 50% or less of the amount of the same functional nucleic acid required to treat the disease or disorder in the absence of the dendrimer.


SEQUENCE LISTING (9 sequences)

SEQ ID NO: 1 - RNA, 21 nt, synthetic construct
  source 1..21: mol_type = other RNA
cattattacttttggtacgcg

SEQ ID NO: 2 - RNA, 26 nt, synthetic construct
  source 1..26: mol_type = other RNA
  misc_feature 1: note = 5' linked to a disulfide thiol-modifier
gcaagctgaccctgaccctgaagttc

SEQ ID NO: 3 - RNA, 26 nt, synthetic construct
  source 1..26: mol_type = other RNA
  misc_feature 1: note = 5' end linked to a disulfide thiol-modifier
  misc_feature 26: note = 3' end linked to a Cy3 dye
gcaagctgaccctgaccctgaagttc

SEQ ID NO: 4 - RNA, 22 nt, synthetic construct
  source 1..22: mol_type = other RNA
tcgtaccgtgagtaataatgcg

SEQ ID NO: 5 - RNA, 22 nt, synthetic construct
  source 1..22: mol_type = other RNA
  misc_feature 22: note = 3' end linked to Thiol-Modifier C6 S-S
cgcattattactcacggtacga

SEQ ID NO: 6 - DNA, 20 bp, synthetic construct
  source 1..20: mol_type = other DNA
ccagtgtgggaagctgtctt

SEQ ID NO: 7 - DNA, 20 bp, synthetic construct
  source 1..20: mol_type = other DNA
aagcaaaagaggaggcaaca

SEQ ID NO: 8 - DNA, 20 bp, synthetic construct
  source 1..20: mol_type = other DNA
agcttcaaatctcgaagcag

SEQ ID NO: 9 - DNA, 20 bp, synthetic construct
  source 1..20: mol_type = other DNA
tgtcctcatcctggaaggtc